SEQUENCE LISTING (7 sequences)

SEQ ID NO: 1 - DNA, 20 bp, Artificial Sequence
  Description of Artificial Sequence Synthetic oligonucleotide
ttcgagcaccgtgtacacac

SEQ ID NO: 2 - DNA, 20 bp, Artificial Sequence
  Description of Artificial Sequence Synthetic oligonucleotide
gtgtgtacacggtgctcgaa

SEQ ID NO: 3 - protein, 591 aa, Homo sapiens
MFGKRKKRVEISAPSNFEHRVHTGFDQHEQKFTGLPRQWQSLIEESARRPKPLVDPACITSIQPGAPKTIVRGSKGAKDGALTLLLDEFENMSVTRSNSLRRDSPPPPARARQENGMPEEPATTARGGPGKAGSRGRFAGHSEAGGGSGDRRRAGPEKRPKSSREGSGGPQESSRDKRPLSGPDVGTPQPAGLASGAKLAAGRPFNTYPRADTDHPSRGAQGEPHDVAPNGPSAGGLAIPQSSSSSSRPPTRARGAPSPGVLGPHASEPQLAPPACTPAAPAVPGPPGPRSPQREPQRVSHEQFRAALQLVVDPGDPRSYLDNFIKIGEGSTGIVCIATVRSSGKLVAVKKMDLRKQQRRELLFNEVVIMRDYQHENVVEMYNSYLVGDELWVVMEFLEGGALTDIVTHTRMNEEQIAAVCLAVLQALSVLHAQGVIHRDIKSDSILLTHDGRVKLSDFGFCAQVSKEVPRRKSLVGTPYWMAPELISRLPYGPEVDIWSLGIMVIEMVDGEPPYFNEPPLKAMKMIRDNLPPRLKNLHKVSPSLKGFLDRLLVRDPAQRATAAELLKHPFLAKAGPPASIVPLMRQNRTR

SEQ ID NO: 4 - DNA, 21 bp, Artificial Sequence
  Description of Artificial Sequence Synthetic oligonucleotide
ccggctggtggccgtcaagaa

SEQ ID NO: 5 - DNA, 21 bp, Artificial Sequence
  Description of Artificial Sequence Synthetic oligonucleotide
cgagaacgtggtggagatgta

SEQ ID NO: 6 - DNA, 20 bp, Artificial Sequence
  Description of Artificial Sequence Synthetic primer
gcacctatcggccataacag

SEQ ID NO: 7 - DNA, 20 bp, Artificial Sequence
  Description of Artificial Sequence Synthetic primer
gccagatacgactggcttgt

(19) United States
(12) Patent Application Publication
(10) Pub. No.: US 2021/0161943 A1
(43) Pub. Date: Jun. 3, 2021

(54) PAK4 INHIBITORS AND METHODS OF USE

(71) Applicant: THE REGENTS OF THE UNIVERSITY OF CALIFORNIA, Oakland, CA (US)

(72) Inventors: Antoni RIBAS, Los Angeles, CA (US); Gabriel Abril RODRIGUEZ, Los Angeles, CA (US); Catherine Grasso, Santa Monica, CA (US); Davis Y. TORREJON, Los Angeles, CA (US)

(21) Appl. No.: 17/048,473

(22) PCT Filed: Apr. 16, 2019
(86) PCT No.: PCT/US2019/027716

§ 371 Date: Oct. 16, 2020

Related U.S. Application Data
(60) Provisional application No. 62/658,136, filed on Apr. 16, 2018.
(60) Provisional application No. 62/743,062, filed on Oct. 9, 2018.

(51) Int. Cl. A61K 31/7105; A61P 35/00; C07K 16/28; A61K 45/06

(52) U.S. Cl. CPC A61K 31/7105; A61P 35/00; A61K 2039/507; C07K 16/2827; A61K 45/06; C07K 16/2818

(57) ABSTRACT

Disclosed herein are methods of treating cancer in a subject, comprising: administering at least one PAK4 inhibitor to the subject; and in certain embodiments administering at least one immunostimulatory agent to the subject. In some aspects, the immunostimulatory agent can be a checkpoint inhibitor. In certain aspects the checkpoint inhibitor can be an anti-PD1 antibody.

### CROSS REFERENCE TO RELATED APPLICATIONS

This application is a national phase application under 35 U.S.C. § 371 of International Application No. PCT/US2019/027716, filed Apr. 16, 2019, which claims priority to U.S. Ser. No. 62/658,136, filed Apr. 16, 2018, and U.S. Ser. No. 62/743,062, filed Oct. 9, 2018, the entire contents of each of which are incorporated herein by reference.

### STATEMENT REGARDING FEDERALLY SPONSORED RESEARCH OR DEVELOPMENT

This invention was made with government support under grants R35 CA197633 and P01 CA 168585 Grant Number CA197633, awarded by the National Institutes of Health. The government has certain rights in the invention.

### SEQUENCE LISTING STATEMENT

The application contains a Sequence Listing which has been submitted in ASCII format and is hereby incorporated by reference in its entirety. Said ASCII copy, created on Feb. 11, 2021, is named UCLA.P0093US_Sequence_Listing_ST25.txt and is 6.68 KB in size.

### BACKGROUND

PAK proteins, a family of serine/threonine p21-activating kinases, include PAK1, PAK2, PAK3 and PAK4. PAK proteins are effectors that link Rho GTPases to cytoskeleton reorganization and nuclear signaling. They serve as targets for the small GTP binding proteins Cdc42 and Rac and have been implicated in a wide range of biological activities. PAK4 interacts specifically with the GTP-bound form of Cdc42Hs and weakly activates the JNK family of MAP kinases. PAK4 is a mediator of filopodia formation and may play a role in the reorganization of the actin cytoskeleton.

PAK4 is a serine/threonine protein kinase that plays a role in a variety of different signaling pathways including cytoskeleton regulation, cell migration, growth, proliferation, or cell survival. Activation by various effectors including growth factor receptors or active CDC42 and RAC1 can result in a conformational change and a subsequent autophosphorylation of PAK4 on several serine and/or threonine residues. PAK4 phosphorylates and inactivates the protein phosphatase SSH1, leading to increased inhibitory phosphorylation of the actin binding/depolymerizing factor cofilin. PAK4 localizes in sub-cellular domains of the cytoplasm and nucleus. PAK4 regulates cytoskeletal remodeling, phenotypic signaling and gene expression, and affects directional motility, invasion, metastasis, and growth. Similar to PAK1, PAK4-signaling dependent cellular functions also regulate both physiologic and disease processes such as cancer.

PAK4 activity and/or expression has been shown to be inhibited by certain PAK4 inhibitors such as KPT-9274, PF-3758309, LCH-7749944, glaucarubinone, KY-04031, KY-04045, 1-phenanthryl-tetrahydroisoquinoline derivatives, (−)-β-hydrastine, Inka1, GL-1196, GNE-2861, and microRNAs such as miR-145, miR-433, and miR-126.

### SUMMARY

Disclosed herein are methods of treating cancer in a subject, comprising: administering at least one PAK4 inhibitor to the subject; and administering at least one immunostimulatory agent to the subject. In some aspects, the immunostimulatory agent is a checkpoint inhibitor. In certain aspects the checkpoint inhibitor is at least one of an anti-PD1 antibody or an anti-PDL1 antibody.

In some aspects, the cancer is PAK4+, the immunostimulatory agent is an antibody that inhibits binding between PD1 and PDL1, and the PAK4 inhibitor is a small molecule.

In some aspects, the PAK4 inhibitor is a small molecule. In some aspects, the small molecule is KPT-9274 or a pharmaceutically acceptable salt thereof. In some aspects, the small molecule is at least one of PF-3758309, IPA-3, FRAX1036, LCH-7749944, glaucarubinone, KY-04031, KY-04045, 1-phenanthryl-tetrahydroisoquinoline derivatives, (−)-β-hydrastine, Inka1, GL-1196, or GNE-2861, or pharmaceutically acceptable salts thereof. In some aspects, the small molecule is PF-3758309 or a pharmaceutically acceptable salt thereof.

In some aspects, the PAK4 inhibitor is a compound Formula (I)

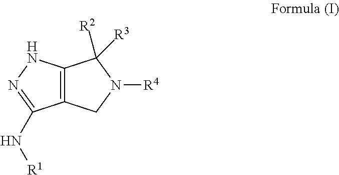

or a pharmaceutically acceptable salt thereof, wherein R¹ is selected from the group consisting of —S(O)R^{a}, —S(O)₂R^{a}, C₁-C₁₂ alkyl, C₁-C₁₂ alkyl substituted by 1 to 6 R⁵, C₃-C₁₂ cycloalkyl, C₃-C₁₂ cycloalkyl substituted by 1 to 6 R⁵, C₂-C₁₂ alkenyl, C₂-C₁₂ alkenyl substituted by 1 to 6 R⁵, C₄-C₁₂ cycloalkenyl, C₄-C₁₂ cycloalkenyl substituted by 1 to 6 R⁵, C₂-C₁₂ alkynyl, C₂-C₁₂ alkynyl substituted by 1 to 6 R⁵, 3-12 membered heterocyclyl, 3-12 membered heterocyclyl substituted by 1 to 6 R⁵, C₁-C₆ aralkyl, C₁-C₆ aralkyl substituted by 1 to 6 R⁵, C₁-C₆ heteroaralkyl, C₁-C₆ heteroaralkyl substituted by 1 to 6 R⁵, phenyl, naphthyl, phenyl substituted by 1 to 6 R⁵, naphthyl substituted by 1 to 6 R⁵, 5-12 member heteroaryl, and 5-12 member heteroaryl substituted by 1 to 6 R⁵, wherein any two adjacent R⁵ together with the atoms to which they are attached may form a fused 4-7 member ring, and the said fused ring is optionally further substituted by 1-3 R^{f}; R² and R³ are each independently selected from the group consisting of —H, C₁-C₆ perfluoroalkyl, C₁-C₆ alkyl, C₃-C₆ cycloalkyl, —(C₁-C₃ alkylene)-(C₃-C₆ cycloalkyl), C₂-C₆ alkenyl, C₂-C₆ alkynyl, C₁-C₆ alkoxy, -(L)ₘ-halide, -(L)ₘ-CN, -(L)ₘ-OH, -(L)ₘ-NH₂, -(L)ₘ-(C₁-C₆ monoalkylamino) and -(L)ₘ-(C₂-C₈ dialkylamino), provided that R² and R³ are not both H; or R² and R³ may form a ring selected from C₃-C₆ cycloalkyl, C₄-C₆ cycloalkenyl and 3-6 member heterocyclyl, the said ring is optionally further substituted by 1 to 2 groups selected from C₁-C₃ alkyl, C₁-C₃ perfluoroalkyl, C₁-C₃ alkoxy, oxo, —(C₁-C₃ alkylene)ₘ-halide, —(C₁-C₃ alkylene)ₘ-CN, —(C₁-C₃ alkylene)ₘ-OH, —(C₁-C₃ alkylene)ₘ-NH₂, —(C₁-C₃ alkylene)ₘ-(C₁-C₆ monoalkylamino) and —(C₁-C₃ alkylene)ₘ-(C₂-C₈ dialkylamino); R⁴ is selected from the group consisting of R^{a}, —C(O)R^{a}, —C(O)NR^{a}R^{b}, —C(O)OR^{a}, —C(O)CH(R^{t})R^{a}, —C(O)NHCH(R^{a})R^{b}, —C(O)OCH(R^{a})R^{b}, —C(O)CH(R^{t})CH(R^{a})R^{b}, —C(O)SR^{a}, —S(O)R^{a}, —S(O)NR^{a}R^{b}, —S(O)OR^{a}, —S(O)₂R^{a}, —S(O)₂NR^{a}R^{b} and —S(O)₂OR^{a}, wherein R^{t} is H or C₁-C₃ alkyl; each R⁵ is independently selected from the group consisting of R^{c}, -(L)ₘ-halide, -(L)ₘ-CN, -(L)ₘ-C(O)R^{c}, -(L)ₘ-C(O)OR^{c}, -(L)ₘ-C(O)NR^{c}R^{d}, -(L)ₘ-C(O)SR^{c}, -(L)ₘ-OR^{c}, -(L)ₘ-OC(O)R^{c}, -(L)ₘ-OC(O)NR^{c}R^{d}, -(L)ₘ-O—C(O)OR^{c}, -(L)ₘ-NO₂, -(L)ₘ-NR^{c}R^{d}, -(L)ₘ-N(R^{c})C(O)R^{d}, -(L)ₘ-N(R^{c})C(O)OR^{d}, -(L)ₘ-NR^{c}S(O)R^{d}, -(L)ₘ-NR^{c}S(O)OR^{d}, -(L)ₘ-NR^{c}S(O)₂R^{d}, -(L)ₘ-NR^{c}S(O)₂OR^{d}, -(L)ₘ-SR^{c}, -(L)ₘ-S(O)R^{c}, -(L)ₘ-S(O)OR^{c}, -(L)ₘ-S(O)₂R^{c}, -(L)ₘ-S(O)₂OR^{c}, -(L)ₘ-S(O)NR^{c}R^{d}, -(L)ₘ-S(O)₂NR^{c}R^{d}, -(L)ₘ-O-L-NR^{c}R^{d}, -(L)ₘ-O-L-OR^{c} and -(L)ₘ-NR^{c}-L-OR^{d}; each R^{a}, R^{b}, R^{c}, and R^{d} is independently selected from the group consisting of H, -(L)ₘ-(C₁-C₆ perfluoroalkyl), C₁-C₁₂ alkyl, —(C₁-C₃ alkylene)ₘ-(C₃-C₁₂ cycloalkyl), —(C₃-C₅ cycloalkylene)ₘ-(C₂-C₁₂ alkenyl), -(L)ₘ-(C₄-C₁₂ cycloakenyl), —(C₃-C₅ cycloalkylene)ₘ-(C₂-C₁₂ alkynyl), -(L)ₘ-(3-12 member heterocyclyl), -(L)ₘ-(phenyl), -(L)ₘ-(naphthyl), and -(L)ₘ-(5-12 member heteroaryl), wherein each R^{a}, R^{b}, R^{c} and R^{d} is independently optionally further substituted by 1-6 R^{f}; R^{a} and R^{b}, or R^{c} and R^{d}, together with the atom to which they are attached, may optionally form a ring selected from 3-12 member heterocyclyl and 5-12 member heteroaryl, the said ring is optionally further substituted by 1-6 R^{f}; each R^{f} is independently selected from oxo, —(C₁-C₃ alkylene)ₘ-(C₁-C₆ perfluoalkyl), C₁-C₁₂ alkyl, C₂-C₆ alkenyl, C₂-C₆ alkynyl, —(C₁-C₃ alkylene)ₘ-(C₃-C₇ cycloalkyl), —(C₁-C₃ alkylene)ₘ-(3-7 member heterocyclyl), —(C₁-C₃ alkylene)ₘ-(5-7 member heteroaryl), -(L)ₘ-halide, -(L)ₘ-CN, -(L)ₘ-C(O)R^{k}, -(L)ₘ-C(O)OR^{k}, -(L)ₘ-C(O)NR^{k}R^{j}, -(L)ₘ-OR^{k}, -(L)ₘ-OC(O)R^{k}, -(L)ₘ-NO₂, -(L)ₘ-NR^{k}R^{j}, -(L)ₘ-N(R^{k})C(O)₁₂³, -(L)ₘ-O-L-NR^{k}R^{j}, -(L)ₘ-SR^{k}, -(L)ₘ-S(O)R^{k}, -(L)ₘ-S(O)₂R^{j}R^{k}, wherein each R^{f} is independently optionally further substituted by 1-3 groups selected from C₁-C₃ alkyl, halide and C₁-C₃ perfluoroalkyl; each R^{k} and R^{j} is independently —H, —OH, C₁-C₃ perfluoroalkyl, C₁-C₆ alkyl, C₂-C₆ alkenyl, C₃-C₆ alkynyl, —(C₁-C₃ alkylene)ₘ-(C₃-C₆ cycloalkyl) or —(C₁-C₃ alkylene)ₘ-(3 to 6 member heterocyclyl), R^{k} and R^{j} may optionally form a ring selected from 3-7 member heterocyclyl and 5-7 member heteroaryl, with said ring optionally further substituted by 1 to 2 groups selected from C₁-C₃ alkyl, C₁-C₃ perfluoroalkyl, C₁-C₃ alkoxy, oxo, —(C₁-C₃ alkylene)ₘ-halide, —(C₁-C₃ alkylene)ₘ-CN, —(C₁-C₃ alkylene)ₘ-OH, —(C₁-C₃ alkylene)ₘ-NH₂, —(C₁-C₃alkylene)ₘ-(C₁-C₆ monoalkylamino) and —(C₁-C₃ alkylene)ₘ-(C₂-C₈ dialkylamino); each L is independently a bivalent radical selected from —(C₁-C₆ alkylene)-, —(C₃-C₇ cycloalkylene)-, —(C₁-C₆ alkylene)-(C₃-C₇ cycloalkylene)- and —(C₃-C₇ cycloalkylene)-(C₁-C₆ alkylene)-; each m is independently 0 or 1; and n is 1, 2, or 3.

In certain embodiments, R¹ is 9 or 10-membered bicyclic heteroaryl (e.g., 9-membered bicyclic heteroaryl) optionally substituted with 1, 2, or 3 independent occurrences of C₁-C₆ alkyl (e.g., 1 occurrence of —CH₃). In certain embodiments, R² and R³ are each independently selected from C₁-C₆ alkyl (e.g., both R² and R³ are —CH₃). In certain embodiments, R⁴ is —C(O)NR^{a}R^{b}. In certain embodiments, R^{a} is —H and R^{b} is -(L)ₘ-(phenyl). In certain embodiments, L is C₁-C₆ alkylene substituted with —NR^{k}R^{j} and m is 1. In certain embodiments, R^{k} and R^{j} are each independently selected from C₁-C₆ alkyl (e.g., both R^{k} and R^{j} are —CH₃). In certain embodiments, R¹ is 9 or 10-membered bicyclic heteroaryl (e.g., 9-membered bicyclic heteroaryl) optionally substituted with 1, 2, or 3 independent occurrences of C₁-C₆ alkyl (e.g., 1 occurrence of —CH₃), R² and R³ are each independently selected from C₁-C₆ alkyl (e.g., both R² and R³ are —CH₃), R⁴ is —C(O)NR^{a}R^{b}, R^{a} is —H and R^{b} is -(L)ₘ-(phenyl), L is C₁-C₆ alkylene substituted with —NR^{k}R^{j} and m is 1, and R^{k} and R^{j} are each independently selected from C₁-C₆ alkyl (e.g., both R^{k} and R^{j} are —CH₃).

In some aspects, the PAK4 inhibitor is a Compound of Formula)

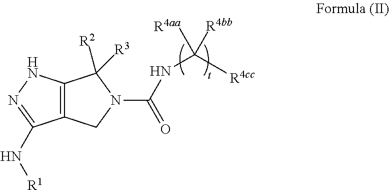

or a pharmaceutically acceptable salt thereof, wherein R¹ is 9 or 10-membered bicyclic heteroaryl optionally substituted with 1, 2, or 3 independent occurrences of C₁-C₆ alkyl (e.g., —CH₃); R² and R³ are each independently selected from C₁-C₆ alkyl (e.g., both R² and R³ are —CH₃); R^{4aa} and R^{4bb} are each independently selected from the group consisting of —H, phenyl, naphthyl, and C₁-C₆ aralkyl; R^{4cc} is —NR^{aa}R^{bb}; R^{aa} and R^{bb} are each independently selected from the group consisting of —H, C₁-C₆ alkyl (e.g., —CH₃), C₂-C₆ alkenyl, C₂-C₆ alkynyl, C₃-C₁₂ cycloalkyl, C₄-C₁₂ cycloalkenyl, 3-12 membered heterocyclyl, and C₁-C₆ aralkyl; and t is an integer selected from the group consisting of 1, 2, and 3. In certain embodiments, R^{aa} and R^{bb} are each independently selected from the group consisting of —H and C₁-C₆ alkyl (e.g., —CH₃).

In certain embodiments, R¹ is

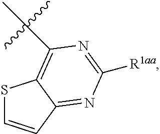

wherein R^{1aa} is C₁-C₆ alkyl (e.g., —CH₃).

In some aspects, the PAK4 inhibitor is an inhibitor that causes a genetic alteration of PAK4 in the cancer, optionally wherein the alteration is a genetic deletion or disruption. In some aspects, the PAK4 inhibitor is a CRISPR-Cas9, a TALEN, a meganuclease, or a zinc-finger nuclease. In some aspects, the PAK4 inhibitor is CRISPR-Cas9. In some aspects, CRISPR-Cas9 comprises PAK4-targeting sgRNAs, optionally wherein the sgRNAs comprise a forward sgRNA having the sequence of 5′-TTCGAGCACCGTGTACACAC-3′ and a reverse sgRNA having the sequence of 5′-GTGTGTACACGGTGCTCGAA-3′. In some aspects, the alteration is a CRISPR-Cas9-induced genetic alteration.

In some aspects, the PAK4 inhibitor is an RNA interference (RNAi) compound or an inhibitor of a microRNA, optionally wherein the microRNA is at least one of miR-145, miR-433, and miR-126.

In some aspects, the immunostimulatory agent comprises a PD1 inhibitor, a PDL1 inhibitor, a CTLA4 inhibitor, a LAG3 inhibitor, a TIM3 inhibitor, a TIGIT inhibitor, a CSF1R inhibitor, a PEGylated cytokine (optionally IL-2, IL-10, IFN), a GITR antibody, a A2AR inhibitor, an IDO inhibitor, or an antibody to OX40, CD40, or CD137/41BB.

In some aspects, the immunostimulatory agent comprises an anti-PD1 antibody, an anti-PDL1 antibody, or an anti-CTLA4 antibody.

In some aspects, the immunostimulatory agent comprises pembrolizumab (Keytruda), nivolumab (Opdivo), atezolizumab (Tecentriq), avelumab (Bavencio), durvalumab (Imfinzi), BMS-936559/MDX1105, PDR001/spartalizumab, GLS-010/AB-122, PF-06801591, BGB-a317, INICSHR-1210, TSR-042, JS-001, LY3300054, ipilimumab (Yervoy), tremelimumab, or AGEN-1884.

In some aspects, the cancer is resistant to treatment with an immunostimulatory agent alone, optionally wherein the immunostimulatory agent is a checkpoint inhibitor.

In some aspects, the cancer is cutaneous melanoma, microsatellite unstable cancers of any histology, head and neck carcinoma, lung carcinoma, renal cell carcinoma, bladder cancer, Merkel cell carcinoma, Hodgkin's lymphoma, gastroesophageal carcinoma, or hepatocellular carcinoma that are resistant to a prior therapy with anti-PD-1, anti-PD-L1, or anti-CTLA4 antibody therapy.

In some aspects, the cancer is a cancer known to have a low likelihood of responding to treatment with a checkpoint inhibitor alone, optionally wherein the cancer is pancreatic cancer, colorectal cancer, breast cancer, prostate cancer, adrenocortical carcinoma, testicular and germinal cell tumors, glioblastoma multiforme, uveal melanoma, thyroid cancer, endometrial cancer, ovarian cancer, cervical carcinoma, cholangiocarcinoma, mesothelioma, thymoma, a lymphoma, a leukemia, multiple myeloma, or a sarcoma.

In some aspects, the cancer is pancreatic cancer, colorectal cancer, breast cancer, adrenocortical carcinoma, testicular and germinal cell tumors, glioblastoma multiforme, uveal melanoma, thyroid cancer, endometrial cancer, ovarian cancer, cervical carcinoma, cholangiocarcinoma, mesothelioma, thymoma, a lymphoma, a leukemia, multiple myeloma or a sarcoma, with the PAK4 inhibitor given together with an immune checkpoint inhibitor along with standard of care chemotherapy and/or radiotherapy.

In some aspects, the cancer is estrogen/progesterone receptor positive breast cancer, or prostate cancer, with the PAK4 inhibitor given together with an immune checkpoint inhibitor and hormone inhibitor therapy.

In some aspects, the cancer is uveal melanoma, with the PAK4 inhibitor given together with an immune checkpoint inhibitor and one or more immune modulators such as a LAG3 inhibitor, a TIM3 inhibitor, a TIGIT inhibitor, a CSF1R inhibitor, a PEGylated cytokine (optionally IL-2, IL-10, IFN), a GITR antibody, a A2AR inhibitor, an IDO inhibitor, or an antibody to OX40, CD40, or CD137/41BB.

In some aspects, the cancer is pancreatic cancer, colorectal cancer, breast cancer, adrenocortical carcinoma, testicular and germinal cell tumors, glioblastoma multiforme, uveal melanoma, thyroid cancer, endometrial cancer, ovarian cancer, cervical carcinoma, cholangiocarcinoma, mesothelioma, thymoma, a lymphoma, a leukemia, multiple myeloma or a sarcoma, with the PAK4 inhibitor given together with an immune checkpoint inhibitor and one or more immune modulators such as a LAG3 inhibitor, a TIM3 inhibitor, a TIGIT inhibitor, a CSF1R inhibitor, a PEGylated cytokine (optionally IL-2, IL-10, IFN), a GITR antibody, a A2AR inhibitor, an IDO inhibitor, or an antibody to OX40, CD40, or CD137/41BB.

In some aspects, the cancer is cutaneous melanoma, microsatellite unstable cancers of any histology, head and neck carcinoma, lung carcinoma, renal cell carcinoma, bladder cancer, Merkel cell carcinoma, Hodgkin's lymphoma, gastroesophageal carcinoma, or hepatocellular carcinoma, with the PAK4 inhibitor given together with an immune checkpoint inhibitor and one or more immune modulators such as a LAG3 inhibitor, a TIM3 inhibitor, a TIGIT inhibitor, a CSF1R inhibitor, a PEGylated cytokine (optionally IL-2, IL-10, IFN), a GITR antibody, a A2AR inhibitor, an IDO inhibitor, or an antibody to OX40, CD40, or CD137/41BB.

In some aspects, the cancer is pancreatic cancer, colorectal cancer, breast cancer, adrenocortical carcinoma, testicular and germinal cell tumors, glioblastoma multiforme, uveal melanoma, thyroid cancer, endometrial cancer, ovarian cancer, cervical carcinoma, cholangiocarcinoma, mesothelioma, thymoma, a lymphoma, multiple myeloma or a sarcoma, with the PAK4 inhibitor given together with an immune checkpoint inhibitor and intratumoral injection of one or more immune stimulating agents such as an oncolytic virus, a TLR agonist, a STING agonist, a RIG-I agonist, or an MDA5 agonist.

In some aspects, the cancer is cutaneous melanoma, microsatellite unstable cancers of any histology, head and neck carcinoma, lung carcinoma, renal cell carcinoma, bladder cancer, Merkel cell carcinoma, Hodgkin's lymphoma, gastroesophageal carcinoma, or hepatocellular carcinoma, with the PAK4 inhibitor given together with an immune checkpoint inhibitor and intratumoral injection of one or more immune stimulating agents such as an oncolytic virus, a TLR agonist, a STING agonist, a RIG-I agonist, or an MDA5 agonist.

In some aspects, the cancer is a lymphoma, a leukemia or multiple myeloma with the PAK4 inhibitor given together with the adoptive cell transfer of T cells modified to express a chimeric antigen receptor (CAR).

In some aspects, the cancer is a solid tumor with the PAK4 inhibitor given together with the adoptive cell transfer of T cells modified to express a transgenic T cell receptor (TCR).

In some aspects, the cancer is a solid tumor with the PAK4 inhibitor given together with the adoptive cell transfer of tumor-infiltrating lymphocytes (TILs).

In some aspects, the cancer is PAK4+.

In some aspects, the subject is a human subject.

Also disclosed herein are methods of treating cancer in a subject, comprising administering a PAK4 inhibitor to the subject, wherein the cancer (1) has been determined to be substantially free or have a low baseline level of tumor-infiltrating T cells; and/or (2) has been determined to have increased PAK4 expression relative to control.

In some aspects, the cancer (1) has been determined to be substantially free of or to have a low baseline level of tumor-infiltrating T cells defined by having a density of less than 500 CD3+ or CD8+ T cells per mm square inside the tumor or at the invasive margin of the tumor when analyzed by immunohistochemistry or by mRNA expression of T cell genes or interferon gamma signaling genes or an equivalent T cell quantitation method; or (2) has been determined to have increased PAK4 expression relative to control, defined by measuring PAK4 protein expression by immunohistochemistry or an equivalent protein quantitation method or PAK4 mRNA expression by RNASeq, Nanostring, or an equivalent mRNA quantitation method.

In some aspects, the subject has received or is concurrently receiving a checkpoint inhibitor. In some aspects, the method further comprises administering a checkpoint inhibitor to the subject. In some aspects, the method further comprises administering a chemotherapy and/or radiotherapy. In some aspects, the method further comprises administering a hormone inhibitor therapy. In some aspects, the method further comprises administering one or more immunostimulatory agents, optionally wherein the agent comprises at least one of a LAG3 inhibitor, a TIM3 inhibitor, a TIGIT inhibitor, a CSF1R inhibitor, a PEGylated cytokine (optionally IL-2, IL-10, IFN), a GITR antibody, a A2AR inhibitor, an IDO inhibitor, or an antibody to OX40, CD40, or CD137/41BB. In some aspects, the method further comprises administering one or more immunostimulating agents, optionally wherein the agent comprises at least one of an oncolytic virus, a TLR agonist, a STING agonist, a RIG-I agonist, or an MDA5 agonist. In some aspects, the method further comprises administering one or more T cells modified to express a chimeric antigen receptor (CAR). In some aspects, the method further comprises administering one or more T cells modified to express a transgenic T cell receptor (TCR). In some aspects, the method further comprises administering one or more tumor-infiltrating lymphocytes (TILs).

In some aspects, the PAK4 inhibitor is a small molecule. In some aspects, the small molecule is KPT-9274 or a pharmaceutically acceptable salt thereof. In some aspects, the small molecule is at least one of PF-3758309, IPA-3, FRAX1036, LCH-7749944, glaucarubinone, KY-04031, KY-040451-phenanthryl-tetrahydroisoquinoline derivatives, (−)-β-hydrastine, Inka GL-1196, or GNE-2861, or pharmaceutically acceptable salts thereof. In some aspects, the small molecule is PF-3758309 or a pharmaceutically acceptable salt thereof.

In some aspects, the PAK4 inhibitor is a compound Formula (I)

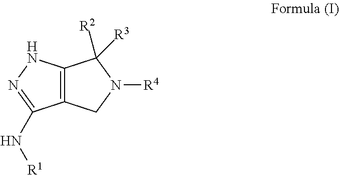

or a pharmaceutically acceptable salt thereof, wherein R¹ is selected from the group consisting of —S(O)R^{a}, —S(O)₂R^{a}, C₁-C₁₂ alkyl, C₁-C₁₂ alkyl substituted by 1 to 6 R⁵, C₃-C₁₂ cycloalkyl, C₃-C₁₂ cycloalkyl substituted by 1 to 6 R⁵, C₂-C₁₂ alkenyl, C₂-C₁₂ alkenyl substituted by 1 to 6 R⁵, C₄-C₁₂ cycloalkenyl, C₄-C₁₂ cycloalkenyl substituted by 1 to 6 R⁵, C₂-C₁₂ alkynyl, C₂-C₁₂ alkynyl substituted by 1 to 6 R⁵, 3-12 membered heterocyclyl, 3-12 membered heterocyclyl substituted by 1 to 6 R⁵, C₁-C₆ aralkyl, C₁-C₆ aralkyl substituted by 1 to 6 R⁵, C₁-C₆ heteroaralkyl, C₁-C₆ heteroaralkyl substituted by 1 to 6 R⁵, phenyl, naphthyl, phenyl substituted by 1 to 6 R⁵, naphthyl substituted by 1 to 6 R⁵, 5-12 member heteroaryl, and 5-12 member heteroaryl substituted by 1 to 6 R⁵, wherein any two adjacent R⁵ together with the atoms to which they are attached may form a fused 4-7 member ring, and the said fused ring is optionally further substituted by 1-3 R^{f}; R² and R³ are each independently selected from the group consisting of —H, C₁-C₆ perfluoroalkyl, C₁-C₆ alkyl, C₃-C₆ cycloalkyl, —(C₁-C₃ alkylene)-(C₃-C₆ cycloalkyl), C₂-C₆ alkenyl, C₂-C₆ alkynyl, C₁-C₆ alkoxy, -(L)ₘ-halide, -(L)ₘ-CN, -(L)ₘ-OH, -(L)ₘ-NH₂, -(L)ₘ-(C₁-C₆ monoalkylamino) and -(L)ₘ-(C₂-C₈ dialkylamino), provided that R² and R³ are not both H; or R² and R³ may form a ring selected from C₃-C₆ cycloalkyl, C₄-C₆ cycloalkenyl and 3-6 member heterocyclyl, the said ring is optionally further substituted by 1 to 2 groups selected from C₁-C₃ alkyl, C₁-C₃ perfluoroalkyl, C₁-C₃ alkoxy, oxo, —(C₁-C₃ alkylene)ₘ-halide, —(C₁-C₃ alkylene)ₘ-CN, —(C₁-C₃ alkylene)ₘ-OH, —(C₁-C₃ alkylene)ₘ-NH₂, —(C₁-C₃ alkylene)ₘ-(C₁-C₆ monoalkylamino) and —(C₁-C₃ alkylene)ₘ-(C₂-C₈ dialkylamino); R⁴ is selected from the group consisting of R^{a}, —C(O)R^{a}, —C(O)NR^{a}R^{b}, —C(O)OR^{a}, —C(O)CH(R^{t})R^{a}, —C(O)NHCH(R^{a})R^{b}, —C(O)OCH(R^{a})R^{b}, —C(O)CH(R^{t})CH(R^{a})R^{b}, —C(O)SR^{a}, —S(O)R^{a}, —S(O)NR^{a}R^{b}, —S(O)OR^{a}, —S(O)₂R^{a}, —S(O)₂NR^{a}R^{b} and —S(O)₂OR^{a}, wherein R^{t} is H or C₁-C₃ alkyl; each R⁵ is independently selected from the group consisting of R^{c}, -(L)ₘ-halide, -(L)ₘ-CN, -(L)ₘ-C(O)R^{c}, -(L)ₘ-C(O)O R^{c}, -(L)ₘ-C(O)NR^{c}R^{d}, -(L)ₘ-C(O)SR^{c}, -(L)ₘ-OR^{c}, -(L)ₘ-OC(O)R^{c}, -(L)ₘ-OC(O)NR^{c}R^{d}, -(L)ₘ-O—C(O)OR^{c}, -(L)ₘ-NO₂, -(L)ₘ-NR^{c}R^{d}, -(L)ₘ-N(R^{c})C(O)R^{d}, -(L)ₘ-N(R^{c})C(O)OR^{d}, -(L)ₘ-NR^{c}S(O)R^{d}, -(L)ₘ-NR^{c}S(O)OR^{d}, -(L)ₘ-NR^{c}S(O)₂R^{d}, -(L)ₘ-NR^{c}S(O)₂OR^{d}, -(L)ₘ-SR^{c}, -(L)ₘ-S(O)R^{c}, -(L)ₘ-S(O)OR^{c}, -(L)ₘ-S(O)₂R^{c}, -(L)ₘ-S(O)₂OR^{c}, -(L)ₘ-S(O)NR^{c}R^{d}, -(L)ₘ-S(O)₂NR^{c}R^{d}, -(L)ₘ-O-L-NR^{c}R^{d}, -(L)ₘ-O-L-OR^{c} and -(L)ₘ-NR^{c}-L-OR^{d}; each R^{a}, R^{b}, R^{c}, and R^{d} is independently selected from the group consisting of H, -(L)ₘ-(C₁-C₆ perfluoroalkyl), C₁-C₁₂ alkyl, —(C₁-C₃ alkylene)ₘ-(C₃-C₁₂ cycloalkyl), —(C₃-C₅ cycloalkylene)ₘ-(C₂-C₁₂ alkenyl), -(L)ₘ-(C₄-C₁₂ cycloakenyl), —(C₃-C₅ cycloalkylene)ₘ-(C₂-C₁₂ alkynyl), -(L)ₘ-(3-12 member heterocyclyl), -(L)ₘ-(phenyl), -(L)ₘ-(naphthyl), and -(L)ₘ-(5-12 member heteroaryl), wherein each R^{a}, R^{b}, R^{c} and R^{d} is independently optionally further substituted by 1-6 R^{f}; R^{a} and R^{b}, or R^{c} and R^{d}, together with the atom to which they are attached, may optionally form a ring selected from 3-12 member heterocyclyl and 5-12 member heteroaryl, the said ring is optionally further substituted by 1-6 R^{f}; each R^{f} is independently selected from oxo, alkylene)ₘ-(C₁-C₆ perfluoalkyl), C₁-C₁₂ alkyl, C₂-C₆ alkenyl, C₂-C₆ alkynyl, alkylene)ₘ-(C₃-C₇ cycloalkyl), alkylene)ₘ-(3-7 member heterocyclyl), alkylene)ₘ-(5-7 member heteroaryl), -(L)ₘ-halide, -(L)ₘ-CN, -(L)ₘ-C(O)R^{k}, -(L)ₘ-C(O)OR^{k}, -(L)ₘ-C(O)NR^{k}R^{j}, -(L)ₘ-OR^{k}, -(L)ₘ-OC(O)R^{k}, -(L)ₘ-NO₂, -(L)ₘ-NR^{k}R^{j}, -(L)ₘ-N(R^{k})C(O)R^{j}, -(L)ₘ-O-L-NR^{k}R^{j}, -(L)ₘ-SR^{k}, -(L)ₘ-S(O)R^{k}, -(L)ₘ-S(O)₂R^{j}R^{k}, wherein each R^{f} is independently optionally further substituted by 1-3 groups selected from C₁-C₃ alkyl, halide and C₁-C₃ perfluoroalkyl; each R^{k} and R^{j} is independently —H, —OH, C₁-C₃ perfluoroalkyl, C₁-C₆ alkyl, C₂-C₆ alkenyl, C₃-C₆ alkynyl, alkylene)ₘ-(C₃-C₆ cycloalkyl) or —(C₁-C₃ alkylene)ₘ-(3 to 6 member heterocyclyl), R^{k} and R^{j} may optionally form a ring selected from 3-7 member heterocyclyl and 5-7 member heteroaryl, with said ring optionally further substituted by 1 to 2 groups selected from C₁-C₃ alkyl, C₁-C₃ perfluoroalkyl, C₁-C₃ alkoxy, oxo, alkylene)ₘ-halide, alkylene)ₘ-CN, alkylene)ₘ-OH, alkylene)ₘ-NH₂, alkylene)ₘ-(C₁-C₆ monoalkylamino) and —(C₁-C₃ alkylene)ₘ-(C₂-C₈ dialkylamino); each L is independently a bivalent radical selected from —(C₁-C₆ alkylene)-, —(C₃-C₇ cycloalkylene)-, alkylene)-(C₃-C₇ cycloalkylene)- and —(C₃-C₇ cycloalkylene)-(C₁-C₆ alkylene)-; each m is independently 0 or 1; and n is 1, 2, or 3.

In certain embodiments, R¹ is 9 or 10-membered bicyclic heteroaryl (e.g., 9-membered bicyclic heteroaryl) optionally substituted with 1, 2, or 3 independent occurrences of C₁-C₆ alkyl (e.g., 1 occurrence of —CH₃). In certain embodiments, R² and R³ are each independently selected from C₁-C₆ alkyl (e.g., both R² and R³ are —CH₃). In certain embodiments, R⁴ is —C(O)NR^{a}R^{b}. In certain embodiments, R^{a} is —H and R^{b} is -(L)ₘ-(phenyl). In certain embodiments, L is C₁-C₆ alkylene substituted with —NR^{k}R^{j} and m is 1. In certain embodiments, R^{k} and R^{j} are each independently selected from C₁-C₆ alkyl (e.g., both R^{k} and R^{j} are —CH₃). In certain embodiments, R¹ is 9 or 10-membered bicyclic heteroaryl (e.g., 9-membered bicyclic heteroaryl) optionally substituted with 1, 2, or 3 independent occurrences of C₁-C₆ alkyl (e.g., 1 occurrence of —CH₃), R² and R³ are each independently selected from C₁-C₆ alkyl (e.g., both R² and R³ are —CH₃), R⁴ is —C(O)NR^{a}R^{b}, R^{a} is —H and R^{b} is -(L)ₘ-(phenyl), L is C₁-C₆ alkylene substituted with —NR^{k}R^{j} and m is 1, and R^{k} and R^{j} are each independently selected from C₁-C₆ alkyl (e.g., both R^{k} and R^{j} are —CH₃).

In some aspects, the PAK4 inhibitor is a compound of Formula (II)

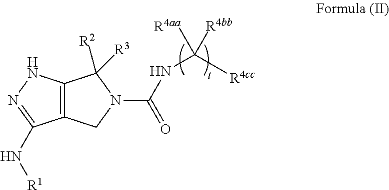

or a pharmaceutically acceptable salt thereof, wherein R¹ is 9 or 10-membered bicyclic heteroaryl optionally substituted with 1, 2, or 3 independent occurrences of C₁-C₆ alkyl (e.g., —CH₃); R² and R³ are each independently selected from C₁-C₆ alkyl (e.g., both R² and R³ are —CH₃); R^{4aa} and R^{4bb} are each independently selected from the group consisting of —H, phenyl, naphthyl, and C₁-C₆ aralkyl; R^{4cc} is —NR^{aa}R^{bb}; R^{aa} and R^{bb} are each independently selected from the group consisting of —H, C₁-C₆ alkyl (e.g., —CH₃), C₂-C₆ alkenyl, C₂-C₆ alkynyl, C₃-C₁₂ cycloalkyl, C₄-C₁₂ cycloalkenyl, 3-12 membered heterocyclyl, and C₁-C₆ aralkyl; and t is an integer selected from the group consisting of 1, 2, and 3. In certain embodiments, R^{aa} and R^{bb} are each independently selected from the group consisting of —H and C₁-C₆ alkyl (e.g., —CH₃).

In certain embodiments, R¹ is

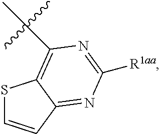

wherein R^{1aa} is C₁-C₆ alkyl (e.g., —CH₃).

In some aspects, the PAK4 inhibitor is an inhibitor that causes a genetic alteration of PAK4 in the cancer, optionally wherein the alteration is a genetic deletion or disruption. In some aspects, the PAK4 inhibitor is a CRISPR-Cas9, a TALEN, a meganuclease, or a zinc-finger nuclease. In some aspects, the PAK4 inhibitor is CRISPR-Cas9. In some aspects, CRISPR-Cas9 comprises PAK4-targeting sgRNAs, optionally wherein the sgRNAs comprise a forward sgRNA having the sequence of 5′-TTCGAGCACCGTGTACACAC-3′ and a reverse sgRNA having the sequence of 5′-GTGTGTACACGGTGCTCGAA-3′. In some aspects, the alteration is a CRISPR-Cas9-induced genetic alteration.

In some aspects, the PAK4 inhibitor is an RNA interference (RNAi) compound or an inhibitor of a microRNA, optionally wherein the microRNA is at least one of miR-145, miR-433, and miR-126.

In some aspects, the immunostimulatory agent comprises a PD1 inhibitor, a PDL1 inhibitor, a CTLA4 inhibitor, a LAG3 inhibitor, a TIM3 inhibitor, a TIGIT inhibitor, a CSF1R inhibitor, a PEGylated cytokine (optionally IL-2, IL-10, IFN), a GITR antibody, a A2AR inhibitor, an IDO inhibitor, or an antibody to OX40, CD40, or CD137/41BB.

In some aspects, the immunostimulatory agent comprises an anti-PD1 antibody, an anti-PDL1 antibody, or an anti-CTLA4 antibody.

In some aspects, the immunostimulatory agent comprises pembrolizumab (Keytruda), nivolumab (Opdivo), atezolizumab (Tecentriq), avelumab (Bavencio), durvalumab (Imfinzi), BMS-936559/MDX1105, PDR001/spartalizumab, GLS-010/AB-122, PF-06801591, BGB-a317, INICSHR-1210, TSR-042, JS-001, LY3300054, ipilimumab (Yervoy), tremelimumab, or AGEN-1884.

In some aspects, the cancer is resistant to treatment with an immunostimulatory agent alone, optionally wherein the immunostimulatory agent is a checkpoint inhibitor.

In some aspects, the cancer is cutaneous melanoma, microsatellite unstable cancers of any histology, head and neck carcinoma, lung carcinoma, renal cell carcinoma, bladder cancer, Merkel cell carcinoma, Hodgkin's lymphoma, gastroesophageal carcinoma, or hepatocellular carcinoma that are resistant to a prior therapy with anti-PD-1, anti-PD-L1, or anti-CTLA4 antibody therapy.

In some aspects, the cancer is a cancer known to have a low likelihood of responding to treatment with a checkpoint inhibitor alone, optionally wherein the cancer is pancreatic cancer, colorectal cancer, breast cancer, prostate cancer, adrenocortical carcinoma, testicular and germinal cell tumors, glioblastoma multiforme, uveal melanoma, thyroid cancer, endometrial cancer, ovarian cancer, cervical carcinoma, cholangiocarcinoma, mesothelioma, thymoma, a lymphoma, a leukemia, multiple myeloma, or a sarcoma.

In some aspects, the cancer is pancreatic cancer, colorectal cancer, breast cancer, adrenocortical carcinoma, testicular and germinal cell tumors, glioblastoma multiforme, uveal melanoma, thyroid cancer, endometrial cancer, ovarian cancer, cervical carcinoma, cholangiocarcinoma, mesothelioma, thymoma, a lymphoma, a leukemia, multiple myeloma or a sarcoma, with the PAK4 inhibitor given together with an immune checkpoint inhibitor along with standard of care chemotherapy and/or radiotherapy.

In some aspects, the cancer is estrogen/progesterone receptor positive breast cancer, or prostate cancer, with the PAK4 inhibitor given together with an immune checkpoint inhibitor and hormone inhibitor therapy.

In some aspects, the cancer is uveal melanoma, with the PAK4 inhibitor given together with an immune checkpoint inhibitor and one or more immune modulators such as a LAG3 inhibitor, a TIM3 inhibitor, a TIGIT inhibitor, a CSF1R inhibitor, a PEGylated cytokine (optionally IL-2, IL-10, IFN), a GITR antibody, a A2AR inhibitor, an IDO inhibitor, or an antibody to OX40, CD40, or CD137/41BB.

In some aspects, the cancer is pancreatic cancer, colorectal cancer, breast cancer, adrenocortical carcinoma, testicular and germinal cell tumors, glioblastoma multiforme, uveal melanoma, thyroid cancer, endometrial cancer, ovarian cancer, cervical carcinoma, cholangiocarcinoma, mesothelioma, thymoma, a lymphoma, a leukemia, multiple myeloma or a sarcoma, with the PAK4 inhibitor given together with an immune checkpoint inhibitor and one or more immune modulators such as a LAG3 inhibitor, a TIM3 inhibitor, a TIGIT inhibitor, a CSF1R inhibitor, a PEGylated cytokine (optionally IL-2, IL-10, IFN), a GITR antibody, a A2AR inhibitor, an IDO inhibitor, or an antibody to OX40, CD40, or CD137/41BB.

In some aspects, the cancer is cutaneous melanoma, microsatellite unstable cancers of any histology, head and neck carcinoma, lung carcinoma, renal cell carcinoma, bladder cancer, Merkel cell carcinoma, Hodgkin's lymphoma, gastroesophageal carcinoma, hepatocellular carcinoma, with the PAK4 inhibitor given together with an immune checkpoint inhibitor and one or more immune modulators such as a LAG3 inhibitor, a TIM3 inhibitor, a TIGIT inhibitor, a CSF1R inhibitor, a PEGylated cytokine (optionally IL-2, IL-10, IFN), a GITR antibody, a A2AR inhibitor, an IDO inhibitor, or an antibody to OX40, CD40, or CD137/41BB.

In some aspects, the cancer is pancreatic cancer, colorectal cancer, breast cancer, adrenocortical carcinoma, testicular and germinal cell tumors, glioblastoma multiforme, uveal melanoma, thyroid cancer, endometrial cancer, ovarian cancer, cervical carcinoma, cholangiocarcinoma, mesothelioma, thymoma, a lymphoma, multiple myeloma or a sarcoma, with the PAK4 inhibitor given together with an immune checkpoint inhibitor and an intratumoral injection of one or more immune stimulating agents such as an oncolytic virus, a TLR agonist, a STING agonist, a RIG-I agonist, or an MDA5 agonist.

In some aspects, the cancer is cutaneous melanoma, microsatellite unstable cancers of any histology, head and neck carcinoma, lung carcinoma, renal cell carcinoma, bladder cancer, Merkel cell carcinoma, Hodgkin's lymphoma, gastroesophageal carcinoma, hepatocellular carcinoma, with the PAK4 inhibitor given together with an immune checkpoint inhibitor and intratumoral injection of one or more immune stimulating agents such as an oncolytic virus, a TLR agonist, a STING agonist, a RIG-I agonist, or an MDA5 agonist.

In some aspects, the cancer is a lymphoma, a leukemia or multiple myeloma with the PAK4 inhibitor given together with the adoptive cell transfer of T cells modified to express a chimeric antigen receptor (CAR).

In some aspects, the cancer is a solid tumor with the PAK4 inhibitor given together with the adoptive cell transfer of T cells modified to express a transgenic T cell receptor (TCR).

In some aspects, the cancer is a solid tumor with the PAK4 inhibitor given together with the adoptive cell transfer of tumor-infiltrating lymphocytes (TILs).

In some aspects, the cancer is PAK4+.

In some aspects, the subject is a human subject.

Also disclosed herein is a kit comprising at least one PAK4 inhibitor, at least one immunostimulatory agent, and instructions for use.

In some aspects, the PAK4 inhibitor is a small molecule. In some aspects, the small molecule is KPT-9274 or a pharmaceutically acceptable salt thereof. In some aspects, the small molecule is at least one of PF-3758309, IPA-3, FRAX1036, LCH-7749944, glaucarubinone. KY-04031. KY-040451-phenanthryl-tetrahydroisoquinoline derivatives. (−)-β-hydrastine, Inka1, GL-1196, or GNE-2861, or pharmaceutically acceptable salts thereof. In some aspects, the small molecule is PF-3758309 or a pharmaceutically acceptable salt thereof.

In some aspects, the PAK4 inhibitor is a compound of Formula (I)

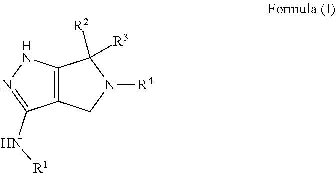

or a pharmaceutically acceptable salt thereof, wherein R¹ is selected from the group consisting of —S(O)R^{a}, —S(O)₂R^{a}, C₁-C₁₂ alkyl, C₁-C₁₂ alkyl substituted by 1 to 6 R⁵, C₃-C₁₂ cycloalkyl, C₃-C₁₂ cycloalkyl substituted by 1 to 6 R⁵, C₂-C₁₂ alkenyl, C₂-C₁₂ alkenyl substituted by 1 to 6 R⁵, C₄-C₁₂ cycloalkenyl, C₄-C₁₂ cycloalkenyl substituted by 1 to 6 R⁵, C₂-C₁₂ alkynyl, C₂-C₁₂ alkynyl substituted by 1 to 6 R⁵, 3-12 membered heterocyclyl, 3-12 membered heterocyclyl substituted by 1 to 6 R⁵, C₁-C₆ aralkyl, C₁-C₆ aralkyl substituted by 1 to 6 R⁵, C₁-C₆ heteroaralkyl, C₁-C₆ heteroaralkyl substituted by 1 to 6 R⁵, phenyl, naphthyl, phenyl substituted by 1 to 6 R⁵, naphthyl substituted by 1 to 6 R⁵, 5-12 member heteroaryl, and 5-12 member heteroaryl substituted by 1 to 6 R⁵, wherein any two adjacent R⁵ together with the atoms to which they are attached may form a fused 4-7 member ring, and the said fused ring is optionally further substituted by 1-3 R^{f}; R² and R³ are each independently selected from the group consisting of —H, C₁-C₆ perfluoroalkyl, C₁-C₆ alkyl, C₃-C₆ cycloalkyl, —(C₁-C₃ alkylene)-(C₃-C₆ cycloalkyl), C₂-C₆ alkenyl, C₂-C₆ alkynyl, C₁-C₆ alkoxy, -(L)ₘ-halide, -(L)ₘ-CN, -(L)ₘ-OH, -(L)ₘ-NH₂, -(L)ₘ-(C₁-C₆ monoalkylamino) and -(L)ₘ-(C₂-C₈ dialkylamino), provided that R² and R³ are not both H; or R² and R³ may form a ring selected from C₃-C₆ cycloalkyl, C₄-C₆ cycloalkenyl and 3-6 member heterocyclyl, the said ring is optionally further substituted by 1 to 2 groups selected from C₁-C₃ alkyl, C₁-C₃ perfluoroalkyl, C₁-C₃ alkoxy, oxo, —(C₁-C₃ alkylene)ₘ-halide, —(C₁-C₃ alkylene)ₘ-CN, —(C₁-C₃ alkylene)ₘ-OH, —(C₁-C₃ alkylene)ₘ-NH₂, —(C₁-C₃ alkylene)ₘ-(C₁-C₆ monoalkylamino) and —(C₁-C₃ alkylene)ₘ-(C₂-C₈ dialkylamino); R⁴ is selected from the group consisting of R^{a}, —C(O)R^{a}, —C(O)NR^{a}R^{b}, —C(O)OR^{a}, —C(O)CH(R^{t})R^{a}, —C(O)NHCH(R^{a})R^{b}, —C(O)OCH(R^{a})R^{b}, —C(O)CH(R^{t})CH(R^{a})R^{b}, —C(O)SR^{a}, —S(O)R^{a}, —S(O)NR^{a}R^{b}, —S(O)OR^{a}, —S(O)₂R^{a}, —S(O)₂NR^{a}R^{b} and —S(O)₂OR^{a}, wherein R^{t} is H or C₁-C₃ alkyl; each R⁵ is independently selected from the group consisting of R^{c}, -(L)ₘ-halide, -(L)ₘ-CN, -(L)ₘ-C(O)R^{c}, -(L)ₘ-C(O)O R^{c}, -(L)ₘ-C(O)NR^{c}R^{d}, -(L)ₘ-C(O)SR^{c}, -(L)ₘ-OR^{c}, -(L)ₘ-OC(O)R^{c}, -(L)ₘ-OC(O)NR^{c}R^{d}, -(L)ₘ-O—C(O)OR^{c}, -(L)ₘ-NO₂, -(L)ₘ-NR^{c}R^{d}, -(L)ₘ-N(R^{c})C(O)R^{d}, -(L)ₘ-N(R^{c})C(O)OR^{d}, -(L)ₘ-NR^{c}S(O)R^{d}, -(L)ₘ-NR^{c}S(O)OR^{d}, -(L)ₘ-NR^{c}S(O)₂R^{d}, -(L)ₘ-NR^{c}S(O)₂OR^{d}, -(L)ₘ-SR^{c}, -(L)ₘ-S(O)R^{c}, -(L)ₘ-S(O)OR^{c}, -(L)ₘ-S(O)₂R^{c}, -(L)ₘ-S(O)₂OR^{c}, -(L)ₘ-S(O)NR^{c}R^{d}, -(L)ₘ-S(O)₂NR^{c}R^{d}, -(L)ₘ-O-L-NR^{c}R^{d}, -(L)ₘ-O-L-OR^{c} and -(L)ₘ-NR^{c}-L-OR^{d}; each R^{a}, R^{b}, R^{c}, and R^{d} is independently selected from the group consisting of H, -(L)ₘ-(C₁-C₆ perfluoroalkyl), C₁-C₁₂ alkyl, —(C₁-C₃ alkylene)ₘ-(C₃-C₁₂ cycloalkyl), —(C₃-C₅ cycloalkylene)ₘ-(C₂-C₁₂ alkenyl), -(L)ₘ-(C₄-C₁₂ cycloakenyl), —(C₃-C₅ cycloalkylene)ₘ-(C₂-C₁₂ alkynyl), -(L)ₘ-(3-12 member heterocyclyl), -(L)ₘ-(phenyl), -(L)ₘ-(naphthyl), and -(L)ₘ-(5-12 member heteroaryl), wherein each R^{a}, R^{b}, R^{c} and R^{d} is independently optionally further substituted by 1-6 R^{f}; R^{a} and R^{b}, or R^{c} and R^{d}, together with the atom to which they are attached, may optionally form a ring selected from 3-12 member heterocyclyl and 5-12 member heteroaryl, the said ring is optionally further substituted by 1-6 R^{f}; each R^{f} is independently selected from oxo, —(C₁-C₃ alkylene)ₘ-(C₁-C₆ perfluoalkyl), C₁-C₁₂ alkyl, C₂-C₆ alkenyl, C₂-C₆ alkynyl, —(C₁-C₃ alkylene)ₘ-(C₃-C₇ cycloalkyl), —(C₁-C₃ alkylene)ₘ-(3-7 member heterocyclyl), —(C₁-C₃ alkylene)ₘ-(5-7 member heteroaryl), -(L)ₘ-halide, -(L)ₘ-CN, -(L)ₘ-C(O)R^{k}, -(L)ₘ-C(O)OR^{k}, -(L)ₘ-C(O)NR^{k}R^{j}, -(L)ₘ-OR^{k}, -(L)ₘ-OC(O)R^{k}, -(L)ₘ-NO₂, -(L)ₘ-NR^{k}R^{j}, -(L)ₘ-N(R^{k})C(O)R^{j}, -(L)ₘ-O-L-NR^{k}R^{j}, -(L)ₘ-SR^{k}, -(L)ₘ-S(O)R^{k}, -(L)ₘ-S(O)₂R^{j}R^{k}, wherein each R^{f} is independently optionally further substituted by 1-3 groups selected from C₁-C₃ alkyl, halide and C₁-C₃ perfluoroalkyl; each R^{k} and R^{j} is independently —H, —OH, C₁-C₃ perfluoroalkyl, C₁-C₆ alkyl, C₂-C₆ alkenyl, C₃-C₆ alkynyl, —(C₁-C₃ alkylene)ₘ-(C₃-C₆ cycloalkyl) or —(C₁-C₃ alkylene)ₘ-(3 to 6 member heterocyclyl), R^{k} and R^{j} may optionally form a ring selected from 3-7 member heterocyclyl and 5-7 member heteroaryl, with said ring optionally further substituted by 1 to 2 groups selected from C₁-C₃ alkyl, C₁-C₃ perfluoroalkyl, C₁-C₃ alkoxy, oxo, —(C₁-C₃ alkylene)ₘ-halide, —(C₁-C₃ alkylene)ₘ-CN, —(C₁-C₃ alkylene)ₘ-OH, —(C₁-C₃ alkylene)ₘ-NH₂, —(C₁-C₃alkylene)ₘ-(C₁-C₆ monoalkylamino) and —(C₁-C₃ alkylene)ₘ-(C₂-C₈ dialkylamino); each L is independently a bivalent radical selected from —(C₁-C₆ alkylene)-, —(C₃-C₇ cycloalkylene)-, —(C₁-C₆ alkylene)-(C₃-C₇ cycloalkylene)- and —(C₃-C₇ cycloalkylene)-(C₁-C₆ alkylene)-; each m is independently 0 or 1; and n is 1, 2, or 3.

In certain embodiments, R¹ is 9 or 10-membered bicyclic heteroaryl (e.g., 9-membered bicyclic heteroaryl) optionally substituted with 1, 2, or 3 independent occurrences of C₁-C₆ alkyl (e.g., 1 occurrence of —CH₃). In certain embodiments, R² and R³ are each independently selected from C₁-C₆ alkyl (e.g., both R² and R³ are —CH₃). In certain embodiments, R⁴ is —C(O)NR^{a}R^{b}. In certain embodiments, R^{a} is —H and R^{b} is -(L)ₘ-(phenyl). In certain embodiments, L is C₁-C₆ alkylene substituted with —NR^{k}R^{j} and m is 1. In certain embodiments, R^{k} and R^{j} are each independently selected from C₁-C₆ alkyl (e.g., both R^{k} and R^{j} are —CH₃). In certain embodiments, R¹ is 9 or 10-membered bicyclic heteroaryl (e.g., 9-membered bicyclic heteroaryl) optionally substituted with 1, 2, or 3 independent occurrences of C₁-C₆ alkyl (e.g., 1 occurrence of —CH₃), R² and R³ are each independently selected from C₁-C₆ alkyl (e.g., both R² and R³ are —CH₃), R⁴ is —C(O)NR^{a}R^{b}, R^{a} is —H and R^{b} is -(L)ₘ-(phenyl), L is C₁-C₆ alkylene substituted with —NR^{k}R^{j} and m is 1, and R^{k} and R^{j} are each independently selected from C₁-C₆ alkyl (e.g., both R^{k} and R^{j} are —CH₃).

In some aspects, the PAK4 inhibitor is a compound of Formula (II)

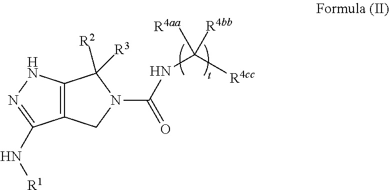

or a pharmaceutically acceptable salt thereof, wherein R¹ is 9 or 10-membered bicyclic heteroaryl optionally substituted with 1, 2, or 3 independent occurrences of C₁-C₆ alkyl (e.g., —CH₃); R² and R³ are each independently selected from C₁-C₆ alkyl (e.g., both R² and R³ are —CH₃); R^{4aa} and R^{4bb} are each independently selected from the group consisting of —H, phenyl, naphthyl, and C₁-C₆ aralkyl; R^{4cc} is —NR^{aa}R^{bb}; R^{aa} and R^{bb} are each independently selected from the group consisting of —H, C₁-C₆ alkyl (e.g., —CH₃), C₂-C₆ alkenyl, C₂-C₆ alkynyl, C₃-C₁₂ cycloalkyl, C₄-C₁₂ cycloalkenyl, 3-12 membered heterocyclyl, and C₁-C₆ aralkyl; and t is an integer selected from the group consisting of 1, 2, and 3. In certain embodiments, R^{aa} and R^{bb} are each independently selected from the group consisting of —H and C₁-C₆ alkyl (e.g., —CH₃).

In certain embodiments, R¹ is

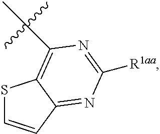

wherein R^{1aa} is C₁-C₆ alkyl (e.g., —CH₃).

In some aspects, the PAK4 inhibitor is an inhibitor that causes a genetic alteration of PAK4 in the cancer, optionally wherein the alteration is a genetic deletion or disruption. In some aspects, the PAK4 inhibitor is a CRISPR-Cas9, a TALEN, a meganuclease, or a zinc-finger nuclease. In some aspects, the PAK4 inhibitor is CRISPR-Cas9. In some aspects, CRISPR-Cas9 comprises PAK4-targeting sgRNAs, optionally wherein the sgRNAs comprise a forward sgRNA having the sequence of 5′-TTCGAGCACCGTGTACACAC-3′ and a reverse sgRNA having the sequence of 5′-GTGTGTACACGGTGCTCGAA -3′. In some aspects, the alteration is a CRISPR-Cas9-induced genetic alteration.

In some aspects, the PAK4 inhibitor is an RNA interference (RNAi) compound or an inhibitor of a microRNA, optionally wherein the microRNA is at least one of miR-145, miR-433, and miR-126.

In some aspects, the immunostimulatory agent comprises a PD1 inhibitor, a PDL1 inhibitor, a CTLA4 inhibitor, a LAG3 inhibitor, a TIM3 inhibitor, a TIGIT inhibitor, a CSF1R inhibitor, a PEGylated cytokine (optionally IL-2, IL-10, IFN), a GITR antibody, a A2AR inhibitor, an IDO inhibitor, or an antibody to OX40, CD40, or CD137/41BB.

In some aspects, the immunostimulatory agent comprises an anti-PD1 antibody, an anti-PDL1 antibody, or an anti-CTLA4 antibody.

In some aspects, the immunostimulatory agent comprises pembrolizumab (Keytruda), nivolumab (Opdivo), atezolizumab (Tecentriq), avelumab (Bavencio), durvalumab (Imfinzi), BMS-936559/MDX1105, PDR001/spartalizumab, GLS-010/AB-122, PF-06801591, BGB-a317, INICSHR-1210, TSR-042, JS-001, LY3300054, ipilimumab (Yervoy), tremelimumab, or AGEN-1884.

In some aspects, the immunostimulatory agent comprises an oncolytic virus, a TLR agonist, a STING agonist, a RIG-I agonist, or an MDA5 agonist. In some aspects, the immunostimulatory agent comprises one or more T cells modified to express a chimeric antigen receptor (CAR). In some aspects, the immunostimulatory agent comprises one or more T cells modified to express a transgenic T cell receptor (TCR). In some aspects, the immunostimulatory agent comprises one or more tumor-infiltrating lymphocytes (TILs).

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1. PAK4 expression negatively correlates with the known immune markers: CD8A (R=−0.54, P=7.95e-06), TNF (R=−0.69, P=1.12e-09), GZMA (R=−0.59, P=7.95e-07), PRF1 (R=−0.41, P=6.20e-04) and the different immune populations assessed using MCP-Counter: T cells (R=−0.62, P=1.04e-07), CD8 T cells (R=−0.55, P=5.25e-06), cytotoxic lymphocytes (R=−0.46, P=1.90e-04) and dendritic cells (R=−0.49, P=6.60e-05).

FIG. 2. Pan-cancer analysis using TCGA transcriptome data shows the negative correlation between PAK4 and T cell, cytotoxic T cell, and dendritic cell score across 32 tumor types including: melanoma, pancreatic cancer and prostate cancer among 10 other tumor types with a P<0.05 for each of the three different immune scores (data not shown). For each TCGA cancer type shown on the x-axis of the figure there are three bars: left is T cell average, while the middle is the abundance of cytotoxic lymphocytes and right is the abundance of myeloid dendritic cells.

FIG. 3. On-treatment non-responding biopsies show higher levels of PAK4 expression (two-sided t-test, P=4.72e-03)

FIG. 4. On-treatment non-responding biopsies are enriched in gene signatures related to known oncogenic signatures involved in immune cell exclusion as observed by GSEA using GO Ontology gene sets as a target. **P<0.01.

FIG. 5. Tumor growth curves for B16 PAK4 KO tumors (n=7 per group) treated with isotype or anti-PD-1. Plotting the mean+/−SD. Anti-PD1 treated B16 PAK4 KO tumors showed decreased tumor growth compared to untreated B16 PAK4 KO tumors (P<0.05 for measurements at day 8, 10, 12 and 14).

FIG. 6. Tumor growth curves for B16 PAK4 KO tumors (n=7 per group) treated with isotype or anti-PD-1. Plotting the individual mouse tumor size. Anti-PD1 treated B16 PAK4 KO tumors showed decreased tumor growth compared to untreated B16 PAK4 KO tumors (P<0.05 for measurements at day 8, 10, 12 and 14).

FIG. 7. Tumor growth curves for B16 WT tumors (n=5 per group) treated with isotype or anti-PD-1. Plotting the mean+/−SD. No significant differences were observed in tumor growth.

FIG. 8. Tumor growth curves for B16 WT tumors (n=5 per group) treated with isotype or anti-PD-1. Plotting the individual mouse tumor size. No significant differences were observed in tumor growth.

FIG. 9. Tumor growth for B16 PAK4 KO tumors with CD8 depletion (n=5, P<0.05 for measurements at day 6, 8, 10, 12 and 14 between B16 PAK4 KO anti-PD-1 and B16 PAK4 KO anti-PD-1 anti-CD8 groups). Plotting the mean+/−SD.

FIG. 10. Tumor growth for B16 PAK4 KO tumors with CD8 depletion (n=5, P<0.05 for measurements at day 6, 8, 10, 12 and 14 between B16 PAK4 KO anti-PD-1 and B16 PAK4 KO anti-PD-1 anti-CD8 groups). Plotting the individual mouse tumor size.

FIG. 11. Percentage of T and NK cell population from CD45 positive cells. KO treated tumors had increased T and NK cell infiltration relative to WT treated tumors (median percentage: 16.18% KO anti-PD-1, 4.99% WT anti-PD-1, P<0.05). KO untreated tumors also showed increased T and NK cell infiltration relative to WT untreated tumors (median percentage: 11.89% KO anti-PD-1, 1.57% WT anti-PD-1, P=0.02).

FIG. 12. Tumor growth curves for B16 WT melanoma tumors treated with KPT-9274 in combination with anti-PD-1 (n=6, purple), KPT-9274 (n=6, green), anti-PD-1 (n=6, red), control (n=6, blue). Combination of KPT-9274 and anti-PD-1 showed decreased tumor growth compared to both anti-PD-1 monotherapy (P=0.01) and KPT-9274 monotherapy (P=0.0007). *P<0.05, **P<0.01, ***P<0.001, ****P<0.0001, ns, not significant.

FIG. 13. Flow cytometry analysis of CD8 positive splenocytes after CD8 depletion. Left panel show splenocytes pattern without anti-CD8 treatment (CD8 population=18.9%) while middle and right panel show splenocytes derived from two independent mice treated with anti-CD8 antibody (CD8 population=0.77% and 0.50% respectively).

FIG. 14. Tumor growth curves for the total of 16 samples used for CyTOF analysis (day 10).

FIG. 15. Percentage of T cell population from CD45 positive cells. B16 PAK4 KO tumors presented increased T cell infiltration than B16 WT tumors (median percentage: 10% KO, 1.37% WT, P=0.009). *P<0.05.

FIG. 16. Growth curves of B16 WT and PAK4 KO cells incubated with murine TNF-α (100 ng/mL). B16 PAK4 KO presented increased inhibition upon TNF-α stimulation (AUC ratio=68.2%) than B16 WT cells (AUC ratio=25.4%).

FIG. 17. Tumor growth curves for MC38 WT tumors treated with PAK4 inhibitor and anti-PD-1 (n=7 for WT PAK4i+anti-PD-1, n=5 for WT PAK4i and WT anti-PD-1 and n=3 for WT isotype group). Plotting the mean+/−SD. Combination of PAK4i and anti-PD-1 or PAK4i monotherapy resulted in significant decreased tumor growth compared to anti-PD-1 alone (P<0.05 for days 7, 10 for both groups and day 14 only with PAK4i+anti-PD-1 combination treatment). PAK4 inhibitor was given twice daily from days 4 to 7 and then discontinued due to PAK4i associated toxicity.

FIG. 18. Tumor growth curves for MC38 WT tumors treated with PAK4 inhibitor and anti-PD-1 (n=7 for WT PAK4i+anti-PD-1, n=5 for WT PAK4i and WT anti-PD-1 and n=3 for WT isotype group). Plotting the individual mouse tumor size. Combination of PAK4i and anti-PD-1 or PAK4i monotherapy resulted in significant decreased tumor growth compared to anti-PD-1 alone (P<0.05 for days 7, 10 for both groups and day 14 only with PAK4i+anti-PD-1 combination treatment). PAK4 inhibitor was given twice daily from days 4 to 7 and then discontinued due to PAK4i associated toxicity.

FIG. 19. Tumor growth curves for MC38 WT and PAK4 KO tumors treated with PD-1 blockade (n=8 for KO ant-PD-1 group, n=7 for KO isotype, and n=4 for WT isotype and WT anti-PD-1 groups). Plotting the mean+/−SD. Treated tumors received four doses of anti-PD-1 in total. Both MC38 PAK4 KO untreated and anti-PD-1 treated tumors showed decreased tumor growth compared to the MC38 WT anti-PD-1 treated group (P<0.05 for days 13, 17 and 21).

FIG. 20. Tumor growth curves for MC38 WT and PAK4 KO tumors treated with PD-1 blockade (n=8 for KO ant-PD-1 group, n=7 for KO isotype, and n=4 for WT isotype and WT anti-PD-1 groups). Plotting the individual mouse tumor size. Treated tumors received four doses of anti-PD-1 in total. Both MC38 PAK4 KO untreated and anti-PD-1 treated tumors showed decreased tumor growth compared to the MC38 WT anti-PD-1 treated group (P<0.05 for days 13, 17 and 21).

FIG. 21. (a)-(d) Plots and Western blots demonstrating generation of multiple, distinct PAK4 KO sublines (6.2, 8.1, and 8.2) of the murine melanoma B16 using CRISPR/Cas9.

FIG. 22. Topflash luciferase activity in B16 WT CRISPR control cells and certain PAK4 KO cell lines and depiction of PAK4 deletion decreasing β-catenin phosphorylation at S675.

FIG. 23. (a)-(d) Plots of Fopflash luciferase activity and β-catenin protein levels in certain B16 PAK4 KO cell lines and B16 WT CC.

FIG. 24. Plots showing anti-tumour activity of PD-1 blockade only in melanoma tumours lacking PAK4 expression in the B16 PAK4 KO 6.2, 8.1, and 8.2 cell lines (FIG. 24 (a), FIG. 24 (b), and FIG. 24(c)) in comparison to a B16 WT control cell line (FIG. 24 (d)).

### DETAILED DESCRIPTION

Terms used in the claims and specification are defined as set forth below unless otherwise specified.

### Chemical Definitions

“Aliphatic” refers to straight-chain, branched or cyclic C₁-C₁₂ hydrocarbons which are completely saturated or which contains one or more units of unsaturation but which are not aromatic. Examples of aliphatic groups include linear, branched or cyclic alkyl, alkenyl, alkynyl groups and hybrids thereof such as (cycloalkyl)alkyl, (cycloalkenyl)alkyl, etc. An aliphatic group may be optionally substituted by 1-6 substituents. Suitable substituents on an aliphatic group include: 3-12 member heterocyclyl, C₆-C₁₀ aryl, 5-12 member heteroaryl, halide, —NO₂, NH₂, NR₂, —CN, —COR, —COOR, —CONR₂, —OH, —OR, —OCOR, —SR, —SOR, —SO₂R, —SONR₂, —SO₂NR₂, wherein R is H, C₁-C₁₀ alkyl, 3-10 member heterocyclyl, C₆-C₁₀ aryl, 5-12 member heteroaryl.

“C₁-C₁₂ alkyl” refers to a straight chain or branched saturated hydrocarbon radical having from 1 to 12 carbon atoms. A C₁-C₁₂ alkyl group may be optionally substituted by at least one substituent. Suitable substituents on a C₁-C₁₂ alkyl group include, but are not limited to, 3-12 member heterocyclyl, C₆-C₁₀ aryl, 5-12 member heteroaryl, halide, —NO₂, —NR₂, —CN, —COR, —COOR, —CONR₂, —OH, —OR, —OCOR, —SR, —SOR, —SO₂R, —SONR₂, —SO₂NR₂, wherein each R is independently selected from the group consisting of —H, C₁-C₁₀ alkyl, 3-12 member heterocyclyl, C₆-C₁₀ aryl, and 5-12 member heteroaryl. Examples of C₁-C₁₂ alkyl groups include, but are not limited to methyl, ethyl, propyl, isopropyl, butyl, sec-butyl, iso-butyl, tert-butyl, pentyl, neo-pentyl, sec-pentyl, hexyl, heptyl, octyl, and the like, including substituted forms thereof. Further, the term “alkyl” refers to a straight chain or branched saturated hydrocarbon radical of 1 to 20 carbon atoms (“C₁-C₂₀ alkyl”), or 1 to 12 carbon atoms (“C₁-C₁₂ alkyl”), or 1 to 8 carbon atoms (“C₁-C₈ alkyl”), or 1 to 6 carbon atoms (“C₁-C₆ alkyl”), or 1 to 4 carbon atoms (“C₁-C₄ alkyl”), or 1 to 3 carbon atoms (“C₁-C₃ alkyl”).

“Cycloalkyl” refers to a cyclic saturated hydrocarbon radical having from 3 to 20 carbon atoms (“C₃-C₂₀ cycloalkyl”), including 3 to 12 carbon atoms (“C₃-C₁₂ cycloalkyl”). A cycloalkyl group may be monocyclic and where permissible may be bicyclic or polycyclic. A cycloalkyl group may be optionally substituted by at least one substituent. Suitable substituents on a cycloalkyl group are the same as those described for an alkyl group. Examples of cycloalkyl groups include, but are not limited to cyclopropyl, cyclobutyl, cyclopentyl, cyclohexyl, cycloheptyl, cyclooctyl, nobornyl, adamantyl, and the like, including substituted forms thereof.

“C₂-C₁₂ alkenyl” refers to a straight chain or branched unsaturated hydrocarbon radical having from 2 to 12 carbon atoms. A C₂-C₁₂ alkenyl group may have one or more points of unsaturation (i.e., one or more carbon-carbon double bonds). In the case where C₂-C₁₂ alkenyl has more than one carbon-carbon double bond, the carbon-carbon double bonds can be conjugated or unconjugated. A C₂-C₁₂ alkenyl group may be optionally substituted by at least one substituent. Suitable substituents on a C₂-C₁₂ alkenyl group are the same as those described for a C₁-C₁₂ alkyl group. Examples of C₂-C₁₂ alkenyl include, but are not limited to, ethenyl, 1-propenyl, 2-propenyl, 1-butenyl, 2-butenyl, iso-butenyl, and the like, including substituted forms thereof. Further, the term “alkenyl” refers to a straight chain or branched unsaturated hydrocarbon radical having from 2 to 20 carbon atoms (“C₂-C₂₀ alkenyl”), or 2 to 12 carbon atoms (“C₂-C₁₂ alkenyl”), or 2 to 8 carbon atoms (“C₂-C₈ alkenyl”), or 2 to 6 carbon atoms (“C₂-C₆ alkenyl”), or 2 to 4 carbon atoms (“C₂-C₄ alkenyl”). An alkenyl group may have one or more points of unsaturation (i.e., one or more carbon-carbon double bonds). In the case where an alkenyl group has more than one carbon-carbon double bond, the carbon-carbon double bonds can be conjugated or unconjugated. An alkenyl group may be substituted or unsubstituted. Suitable substituents on an alkenyl group are the same as those described for a C₁-C₁₂ alkyl group.

“Alkoxy” refers to Ole, wherein R¹ is C₁-C₁₂ alkyl, C₂-C₁₂ alkenyl, C₂-C₁₂ alkynyl, C₃-C₁₂ cycloalkyl or (C₁-C₆ alkylene)-(C₃-C₁₂ cycloalkyl). A “C₁-C₁₂ alkoxy” refers to an alkoxy group, as defined herein, wherein R^{c1} has 1 to 12 total carbon atoms.

“Alkylamino” refers to —NR^{p}R^{q} wherein each R^{p} and R^{q} is independently H, C₁-C₁₂ alkyl, C₂-C₁₂ alkenyl, C₂-C₁₂ alkynyl, C₃-C₁₂ cycloalkyl, (C₁-C₆ alkylene)-(C₃-C₁₂ cycloalkyl) provided R^{p} and R^{q} are not both H. A “monoalkylamino” refers to an alkylamino group, as defined herein, wherein one of R^{p} and R^{q} is H. A “dialkylamino” refers to an alkylamino group, as defined herein, wherein none of R^{p} and R^{q} is H. A “C₂-C₈ dialkylamino” refers to a dialkylamino group that contains 2 to 8 carbon atoms. A “C₁-C₆ monoalkylamino” refers to a monoalkylamino group that contains 1 to 6 carbon atoms.

“C₂-C₁₂ alkynyl” refers to a straight chain or branched hydrocarbon radical having from 2-12 carbon atoms and at least one carbon-carbon triple bond. In the case where C₂-C₁₂ alkynyl has more than one carbon-carbon double bond, the carbon-carbon double bonds can be conjugated or unconjugated. A C₂-C₁₂ alkynyl group may be optionally substituted by at least one substituent. Suitable substituents on a C₂-C₁₂ alkynyl group are the same as those described for a C₁-C₁₂ alkyl group. Examples of C₂-C₁₂ alkynyl include, but are not limited to, ethynyl, 1-propynyl, 2-propynyl, 1-butynyl, 2-butynyl, and the like, including substituted forms thereof. Further, the term “alkynyl” refers to a straight chain or branched hydrocarbon radical of 2 to 20 carbon atoms (“C₂-C₂₀ alkynyl”), or 2 to 12 carbon atoms (“C₂-C₁₂ alkynyl”), or 2 to 8 carbon atoms (“C₂-C₈ alkynyl”), or 2 to 6 carbon atoms (“C₂-C₆ alkynyl”), or 2 to 4 carbon atoms (“C₂-C₄ alkynyl”), and having at least one carbon-carbon triple bond. Alkynyl may be substituted or unsubstituted. Suitable substituents on an alkynyl group are the same as those described for a C₁-C₁₂ alkyl group.

The term “aryl” refers to an all-carbon monocyclic ring or polycyclic ring of 6 to 20 carbon atoms having a completely conjugated pi-electron system. Examples of aryl include but are not limited to phenyl, naphthyl, and anthracenyl. C₆-C₁₀ aryl refers to aryl with 6-10 carbon atoms in the cyclic structure, including phenyl and naphthyl.

“Aralkyl” refers to alkyl, as defined herein, that is substituted with an C₆-C₁₀ aryl group as defined above; e.g., —CH₂-phenyl, —CH₂CH₂-phenyl, —CH₂CH₂CH₂-phenyl, —CH₃CH(CH₃)CH₂-phenyl, and the like and derivatives thereof. A C₁-C₆ aralkyl refers to a C₁-C₆ alkyl that is substituted with a C₆-C₁₀ aryl group.

“Heteroaralkyl” group means alkyl, as defined herein, that is substituted with a 5-12 membered heteroaryl group; e.g., —CH₂-pyridinyl, —CH₂CH₂-pyrimidinyl, —CH₂CH₂CH₂-imidazolyl, and the like, and derivatives thereof. A C₁-C₆ heteroaralkyl refers to a C₁-C₆ alkyl that is substituted with an 5-12 membered heteroaryl group.

“Heteroaryl” refers to a monocyclic or fused ring group containing one, two, three or four ring heteroatoms selected from N, O, and S, the remaining ring atoms being C, and, in addition, having a completely conjugated pi-electron system. Examples, without limitation, of unsubstituted heteroaryl groups are pyrrole, furan, thiophene, imidazole, oxazole, thiazole, pyrazole, pyridine, pyrimidine, quinoline, isoquinoline, purine, tetrazole, triazine, and carbazole. The heteroaryl group may be substituted or unsubstituted. Typical substituents include C₁-C₁₂ aliphatic, 3-10 membered heterocyclyl, 6-10 membered aryl, halide, —NO₂, NH₂, NR₂, —CN, —COR, —COOR, —CONR₂, —OH, —OR, —OCOR, —SR, —SOR, —SO₂R, —SONR₂, —SO₂NR₂, wherein R is a C₁-C₁₀ aliphatic, 3-10 membered heterocyclyl, C₆-C₁₀ aryl, and 5-10 membered heteroaryl.

Examples of typical monocyclic heteroaryl groups include, but are not limited to: pyrrolyl, furanyl, thiophenyl, pyrazolyl, imidazolyl, isoxazolyl, oxazolyl, isothiazolyl, thiazolyl, 1,2,3-triazolyl, 1,3,4-triazolyl, 1-oxa-2,3,-diazolyl, 1-oxa-2,4-dizolyl, 1-oxa-2,5-diazolyl, 1-oxa-3,4-diazolyl, 1-thia-3,4-diazolyl, 1-thia-2,3-diazolyl, 1-thia-2,4,-diazolyl, 1-thia-2,5-diazolyl, 1-thia-3,4-diazolyl, tetrazolyl, pyridinyl, pyridazinyl, pyrimidinyl, pyrazinyl, and triazinyl.

Examples of bicyclic heteroaryl groups include, but are not limited to: benzofuranyl, benzothiophenyl, indolyl, benzimidazolyl, indazolyl, benzotriazolyl, pyrrolo[2,3-b]pyridinyl, pyrrolo[2,3-c]pyridinyl, pyrrolo[3,2-c]pyridinyl, pyrrolo[3,2-b]pyridinyl, imidazo[4,5-b]pyridinyl, imidazo[4,5-c]pyridinyl, pyrazolo[4,3-d]pyridinyl, pyrazolo[4,3-c]pyridinyl, pyrazolo[3,4-c]pyridinyl, pyrazolo[3,4-b]pyridinyl, isoindolyl, indazolyl, purinyl, indolininyl, imidazo[1,2-a]pyridinyl, imidazo[1,5-a]pyridinyl, pyrazolo[1,5-a]pyridinyl, pyrrolo[1,2-b]pyridazinyl, imidazo[1,2-c]pyrimidinyl, thienopyrimidinyl, quinolinyl, isoquinolinyl, cinnolinyl, azaquinazoline, quinoxalinyl, phthalazinyl, 1,6-naphthyridinyl, 1,7-naphthyridinyl, 1,8-naphthyridinyl, 1,5-naphthyridinyl, 2,6-naphthyridinyl, 2,7-naphthyridinyl, pyrido[3,2-d]pyrimidinyl, pyrido[4,3-d]pyrimidinyl, pyrido[3,4-d]pyrimidinyl, pyrido[2,3-d]pyrimidinyl, pyrido[2,3-b]pyrazinyl, pyrido[3,4-b]pyrazinyl, pyrimido[5,4-d]pyrimidinyl, pyrazino[2,3-b]pyrazinyl, and pyrimido[4,5-d]pyrimidinyl.

“Heteroalicyclic” or “heterocyclyl” refers to a monocyclic or polycyclic group having from 3 to 12 ring atoms, wherein from 1 to 4 ring atoms are heteroatoms selected from N, O, and S. “Heteroalicyclic” or “heterocyclyl” may also have one or more double bonds. However, “Heteroalicyclic” or “heterocyclyl” do not have a completely conjugated pi-electron system. “Heteroalicyclic” or “heterocyclyl” can be substituted or unsubstituted. Typical substituents include, but are not limited to, C₁-C₁₂ aliphatic, 6-10 membered aryl, 6-10 membered aryl, halide, —NO₂, —NH₂, —NR₂, —CN, —COR, —COOR, —CONR₂, —OH, —OR, —OCOR, —SR, —SOR, —SO₂R, wherein R is a C₁-C₁₀ alkyl, 3-10 member heterocyclyl, C₆-C₁₀ aryl, and 5-10 membered heteroaryl.

Examples of saturated heterocyclyl groups include, but are not limited to: oxiranyl, thiaranyl, aziridinyl, oxetanyl, thiatanyl, azetidinyl, tetrahydrofuranyl, tetrahydrothiophenyl, pyrrolidinyl, tetrahydropyranyl, piperidinyl, 1,4-dioxanyl, 1,4-oxathianyl, morpholinyl, 1,4-dithianyl, piperazinyl, 1,4-azathianyl, oxepanyl, thiepanyl, azepanyl, 1,4-dioxepanyl, 1,4-oxathiepanyl, 1,4-oxaazepanyl, 1,4-dithiepanyl, 1,4-thieazepanyl, 1,4-diazepanyl, and tropanyl.

Examples of partially unsaturated heterocyclyl groups include, but are not limited to: 3,4-dihydro-2H-pyranyl, 5,6-dihydro-2H-pyranyl, 2H-pyranyl, 1,2,3,4-tetrahydropyrdinyl, and 1,2,5,6-tetrahydropyridinyl.

“Lower alkyl” refers to alkyl containing 1, 2, 3, or 4 carbon atoms and may be branched or linear. Suitable substituents on a lower alkyl group are the same as those described for a C₁-C₁₂ alkyl group.

When “ene” is added after “yl” at the end a term to form a new term, the new term refers to a diradical formed by removing one hydrogen atom from the original term of which the new term derived from. For example, an alkylene refers to a diradical group formed by removing one hydrogen atom from an alkyl group and that a “methylene” refers to a divalent radical —CH₂— derived from removing one hydrogen atom from methyl. More examples of such diradicals include, but are not limited to: alkenylene, alkynylene, cycloalkylene, phenylene, heterocyclylene, and heteroarylene, which are derived from alkenyl, alkynyl, cycloalkyl, phenyl, heterocyclyl, and heteroaryl, respectively. For example, “C₁-C₃ alkylene” refers to all of the following: —CH₂—, —CH₂CH₂—, —CH₂CH₂CH₂—, —CH(CH₃)CH₂— and —CH(CH₂CH₃)—.

“Oxo” refers to an oxygen double bond substitution (i.e., ═O).

“Perfluoroalkyl” refers to an alkyl group in which all of its hydrogen atoms are replaced by fluorine atoms. For example, C₁-C₃ perfluoroalkyl refers to a perfluoroalkyl group containing 1 to 3 carbon atoms.

The term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts of the compounds of described herein (e.g., a compound of Formula (I)) include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxyethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

### Other Definitions

The term “ameliorating” refers to any therapeutically beneficial result in the treatment of a disease state, e.g., a PAK4+ disease state, including prophylaxis, lessening in the severity or progression, remission, or cure thereof.

The term “in situ” refers to processes that occur in a living cell growing separate from a living organism, e.g., growing in tissue culture.

The term “in vivo” refers to processes that occur in a living organism.

The term “mammal” as used herein includes both humans and non-humans and include but is not limited to humans, non-human primates, canines, felines, murines, bovines, equines, and porcines.

The term “antibody” is used herein in its broadest sense and includes certain types of immunoglobulin molecules comprising one or more antigen-binding domains that specifically bind to an antigen or epitope. An antibody specifically includes intact antibodies (e.g., intact immunoglobulins), antibody fragments such as antigen-binding fragments of antibodies, and multi-specific antibodies. One example of an antigen-binding domain is an antigen-binding domain formed by a V_{H}-V_{L} dimer. An antibody is one type of antigen binding protein. V_{H} and V_{L} regions may be further subdivided into regions of hypervariability (“hypervariable regions (HVRs);” also called “complementarity determining regions” (CDRs)) interspersed with regions that are more conserved. The more conserved regions are called framework regions (FRs). Each V_{H} and V_{L} generally comprises three CDRs and four FRs, arranged in the following order (from N-terminus to C-terminus): FR1-CDR1-FR2-CDR2-FR3-CDR3-FR4. The CDRs are involved in antigen binding, and influence antigen specificity and binding affinity of the antibody. See Kabat et al., Sequences of Proteins of Immunological Interest 5th ed. (1991) Public Health Service, National Institutes of Health, Bethesda, Md., incorporated by reference in its entirety. An “antibody fragment” comprises a portion of an intact antibody, such as the antigen-binding or variable region of an intact antibody. Antibody fragments include, for example, Fv fragments, Fab fragments, F(ab′)₂ fragments, Fab′ fragments, scFv (sFv) fragments, and scFv-Fc fragments.

The term “cytotoxic agent,” as used herein, refers to a substance that inhibits or prevents a cellular function and/or causes cell death or destruction.

A “chemotherapeutic agent” refers to a chemical compound useful in the treatment of cancer. Chemotherapeutic agents include “anti-hormonal agents” or “endocrine therapeutics” which act to regulate, reduce, block, or inhibit the effects of hormones that can promote the growth of cancer.

The term “cytostatic agent” refers to a compound or composition which arrests growth of a cell either in vitro or in vivo. In some embodiments, a cytostatic agent is an agent that reduces the percentage of cells in S phase. In some embodiments, a cytostatic agent reduces the percentage of cells in S phase by at least about 20%, at least about 40%, at least about 60%, or at least about 80%.

The term “tumor” refers to all neoplastic cell growth and proliferation, whether malignant or benign, and all pre-cancerous and cancerous cells and tissues. The terms “cancer,” “cancerous,” “cell proliferative disorder,” “proliferative disorder” and “tumor” are not mutually exclusive as referred to herein. The terms “cell proliferative disorder” and “proliferative disorder” refer to disorders that are associated with some degree of abnormal cell proliferation. In some embodiments, the cell proliferative disorder is a cancer.

The term “pharmaceutical composition” refers to a preparation which is in such form as to permit the biological activity of an active ingredient contained therein to be effective in treating a subject, and which contains no additional components which are unacceptably toxic to the subject.

The terms “modulate” and “modulation” refer to reducing or inhibiting or, alternatively, activating or increasing, a recited variable.

The term “sufficient amount” means an amount sufficient to produce a desired effect, e.g., an amount sufficient to modulate protein aggregation in a cell.

The term “therapeutically effective amount” is an amount that is effective to ameliorate a symptom of a disease.

It must be noted that, as used in the specification and the appended claims, the singular forms “a,” “an” and “the” include plural referents unless the context clearly dictates otherwise.

### PAK4 and PAK4 Inhibition

PAK proteins, a family of serine/threonine p21-activating kinases, include PAK1, PAK2, PAK3, and PAK4. PAK proteins are effectors that link Rho GTPases to cytoskeletal reorganization and nuclear signaling. They serve as targets for the small GTP binding proteins Cdc42 and Rac and have been implicated in a wide range of biological activities. PAK4 interacts specifically with the GTP-bound form of Cdc42Hs and weakly activates the JNK family of MAP kinases. PAK4 is a mediator of filopodia formation and may play a role in the reorganization of the actin cytoskeleton. Multiple alternatively spliced transcript variants encoding distinct isoforms have been found for this gene. PAK4 has been shown to be repressed at the translational level by miR-24.

PAK4 regulates cellular processes by its scaffolding activity and/or by phosphorylation of effector substrates, which in-turn, set-up a cascades of biochemical events cumulating into a cellular phenotypic response. Examples of PAK4-regulated cellular processes include, dynamic reorganization of actin, and microtubule fibers, anchorage-independent growth, filopodium formation, and cell motility.

PAK4 is also known as p21 (RAC1) activated kinase 4. The RefSeq for human PAK4 can be found at accession number NM 001014831.2 on the NCBI website on Apr. 9, 2018. The amino acid sequence of PAK4 is shown in the table below.

| Name | Amino Acid Sequence |
|---|---|
| PAM | MFGKRKKRVE ISAPSNFEHR VHTGFDQHEQ |
| (human) | KFTGLPRQWQ SLIEESARRP KPLVDPACIT |
| | SIQPGAPKTI VRGSKGAKDG ALTLLLDEFE |
| | NMSVTRSNSL RRDSPPPPAR ARQENGMPEE |
| | PATTARGGPG KAGSRGRFAG HSEAGGGSGD |
| | RRRAGPEKRP KSSREGSGGP QESSRDKRPL |
| | SGPDVGTPQP AGLASGAKLA AGRPFNTYPR |
| | ADTDHPSRGA QGEPHDVAPN GPSAGGLAIP |
| | QSSSSSSRPP TRARGAPSPG VLGPHASEPQ |
| | LAPPACTPAA PAVPGPPGPR SPQREPQRVS |
| | HEQFRAALQL VVDPGDPRSY LDNFIKIGEG |
| | STGIVCIATV RSSGKLVAVK KMDLRKQQRR |
| | ELLFNEVVIM RDYQHENVVE MYNSYLVGDE |
| | LWVVMEFLEG GALTDIVTHT RMNEEQIAAV |
| | CLAVLQALSV LHAQGVIHRD IKSDSILLTH |
| | DGRVKLSDFG FCAQVSKEVP RRKSLVGTPY |
| | WMAPELISRL PYGPEVDIWS LGIMVIEMVD |
| | GEPPYFNEPP LKAMKMIRDN LPPRLKNLHK |
| | VSPSLKGFLD RLLVRDPAQR ATAAELLKHP |
| | FLAKAGPPAS IVPLMRQNRT R |

Certain PAK4 inhibitors have been identified previously and are known in the art. PAK4 inhibitors can include small molecules, RNAi agents such as siRNA, and gene editing agents such as CRISPR-Cas9.

Exemplary PAK4 inhibitors include: KPT-9274, PF-3758309, LCH-7749944, glaucarubinone, KY-04031, KY-04045, 1-phenanthryl-tetrahydroisoquinoline derivatives, (−)-β-hydrastine, Inka1, GL-1196, GNE-2861, and microRNAs such as miR-145, miR-433, and miR-126. These and other PAK4 inhibitors are summarized in further detail below.

KPT-9274 is an exemplary PAK4 inhibitor. Rane et al., “A novel orally bioavailable compound KPT-9274 inhibits PAK4, and blocks triple negative breast cancer tumor growth.” Sci Rep. 2017 Feb. 15; 7:42555. doi: 10.1038/srep42555.

The structure of KPT-9274 is shown below:

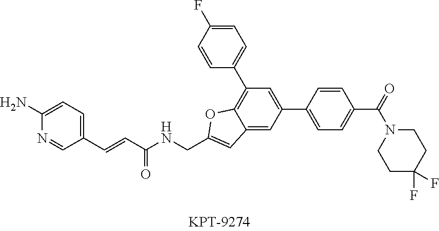 [SMILES: Nc1ccc(/C=C/C(=O)NCc2cc3cc(-c4ccc(C(=O)N5CCC(F)(F)CC5)cc4)cc(-c4ccc(F)cc4)c3o2)cn1]

PF-3758309 is an exemplary PAK4 inhibitor. Murray B W, Guo C, Piraino J, Westwick J K, Zhang C, Lamerdin J, Dagostino E, Knighton D, Loi C M, Zager M, Kraynov E, Popoff I, Christensen J G, Martinez R, Kephart S E, Marakovits J, Karlicek S, Bergqvist S, Smeal T (May 2010). “Small-molecule p21-activated kinase inhibitor PF-3758309 is a potent inhibitor of oncogenic signaling and tumor growth”. Proceedings of the National Academy of Sciences of the United States of America. 107 (20): 9446-51.

The structure of PF-3758309 is shown below:

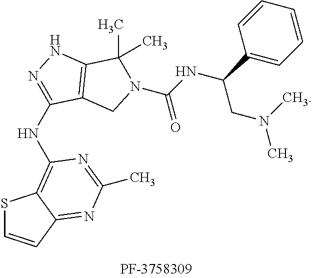 [SMILES: Cc1nc(Nc2n[nH]c3c2CN(C(=O)N[C@H](CN(C)C)c2ccccc2)C3(C)C)c2sccc2n1]

Additional exemplary PAK4 inhibitors are disclosed in U.S. Pat. No. 8,530,652, which is incorporated herein by reference.

In some aspects, the PAK4 inhibitor is KPT-9274 or a pharmaceutically acceptable salt thereof. In some aspects, the PAK4 inhibitor is at least one of PF-3758309, IPA-3, FRAX1036, LCH-7749944, glaucarubinone, KY-04031, KY-04045, 1-phenanthryl-tetrahydroisoquinoline derivatives, (−)-β-hydrastine, Inka1, GL-1196, or CINE-2861, or pharmaceutically acceptable salts thereof. In some aspects, the PAK4 inhibitor is PF 3758309 or a pharmaceutically acceptable salt thereof.

In some aspects, the PAK4 inhibitor is a compound of For nu (I)

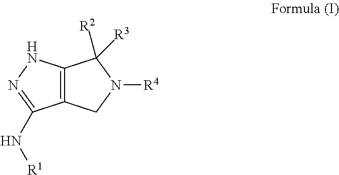

or a pharmaceutically acceptable salt thereof, wherein R¹ is selected from the group consisting of —S(O)R^{a}, —S(O)₂R^{a}, C₁-C₁₂ alkyl, C₁-C₁₂ alkyl substituted by 1 to 6 R⁵, C₃-C₁₂ cycloalkyl, C₃-C₁₂ cycloalkyl substituted by 1 to 6 R⁵, C₂-C₁₂ alkenyl, C₂-C₁₂ alkenyl substituted by 1 to 6 R⁵, C₄-C₁₂ cycloalkenyl, C₄-C₁₂ cycloalkenyl substituted by 1 to 6 R⁵, C₂-C₁₂ alkynyl, C₂-C₁₂ alkynyl substituted by 1 to 6 R⁵, 3-12 membered heterocyclyl, 3-12 membered heterocyclyl substituted by 1 to 6 R⁵, C₁-C₆ aralkyl, C₁-C₆ aralkyl substituted by 1 to 6 R⁵, C₁-C₆ heteroaralkyl, C₁-C₆ heteroaralkyl substituted by 1 to 6 R⁵, phenyl, naphthyl, phenyl substituted by 1 to 6 R⁵, naphthyl substituted by 1 to 6 R⁵, 5-12 member heteroaryl, and 5-12 member heteroaryl substituted by 1 to 6 R⁵, wherein any two adjacent R⁵ together with the atoms to which they are attached may form a fused 4-7 member ring, and the said fused ring is optionally further substituted by 1-3 R^{f}; R² and R³ are each independently selected from the group consisting of —H, C₁-C₆ perfluoroalkyl, C₁-C₆ alkyl, C₃-C₆ cycloalkyl, —(C₁-C₃ alkylene)-(C₃-C₆ cycloalkyl), C₂-C₆ alkenyl, C₂-C₆ alkynyl, C₁-C₆ alkoxy, -(L)ₘ-halide, -(L)ₘ-CN, -(L)ₘ-OH, -(L)ₘ-NH₂, -(L)ₘ-(C₁-C₆ monoalkylamino) and -(L)ₘ-(C₂-C₈ dialkylamino), provided that R² and R³ are not both H; or R² and R³ may form a ring selected from C₃-C₆ cycloalkyl, C₄-C₆ cycloalkenyl and 3-6 member heterocyclyl, the said ring is optionally further substituted by 1 to 2 groups selected from C₁-C₃ alkyl, C₁-C₃ perfluoroalkyl, C₁-C₃ alkoxy, oxo, —(C₁-C₃ alkylene)ₘ-halide, —(C₁-C₃ alkylene)ₘ-CN, —(C₁-C₃ alkylene)ₘ-OH, —(C₁-C₃ alkylene)ₘ-NH₂, —(C₁-C₃ alkylene)ₘ-(C₁-C₆ monoalkylamino) and —(C₁-C₃ alkylene)ₘ-(C₂-C₈ dialkylamino); R⁴ is selected from the group consisting of R^{a}, —C(O)R^{a}, —C(O)NR^{a}R^{b}, —C(O)OR^{a}, —C(O)CH(R^{t})R^{a}, —C(O)NHCH(R^{a})R^{b}, —C(O)OCH(R^{a})R^{b}, —C(O)CH(R^{t})CH(R^{a})R^{b}, —C(O)SR^{a}, —S(O)R^{a}, —S(O)NR^{a}R^{b}, —S(O)OR^{a}, —S(O)₂R^{a}, —S(O)₂NR^{a}R^{b} and —S(O)₂OR^{a}, wherein R^{t} is H or C₁-C₃ alkyl; each R⁵ is independently selected from the group consisting of R^{c}, -(L)ₘ-halide, -(L)ₘ-CN, -(L)ₘ-C(O)R^{c}, -(L)ₘ-C(O)O R^{c}, -(L)ₘ-C(O)NR^{c}R^{d}, -(L)ₘ-C(O)SR^{c}, -(L)ₘ-OR^{c}, -(L)ₘ-OC(O)R^{c}, -(L)ₘ-OC(O)NR^{c}R^{d}, -(L)ₘ-O—C(O)OR^{c}, -(L)ₘ-NO₂, -(L)ₘ-NR^{c}R^{d}, -(L)ₘ-N(R^{c})C(O)R^{d}, -(L)ₘ-N(R^{c})C(O)OR^{d}, -(L)ₘ-NR^{c}S(O)R^{d}, -(L)ₘ-NR^{c}S(O)OR^{d}, -(L)ₘ-NR^{c}S(O)₂R^{d}, -(L)ₘ-NR^{c}S(O)₂OR^{d}, -(L)ₘ-SR^{c}, -(L)ₘ-S(O)R^{c}, -(L)ₘ-S(O)OR^{c}, -(L)ₘ-S(O)₂R^{c}, -(L)ₘ-S(O)₂OR^{c}, -(L)ₘ-S(O)NR^{c}R^{d}, -(L)ₘ-S(O)₂NR^{c}R^{d}, -(L)ₘ-O-L-NR^{c}R^{d}, -(L)ₘ-O-L-OR^{c} and -(L)ₘ-NR^{c}-L-OR^{d}; each R^{a}, R^{b}, R^{c}, and R^{d} is independently selected from the group consisting of H, -(L)ₘ-(C₁-C₆ perfluoroalkyl), C₁-C₁₂ alkyl, —(C₁-C₃ alkylene)ₘ-(C₃-C₁₂ cycloalkyl), —(C₃-C₅ cycloalkylene)ₘ-(C₂-C₁₂ alkenyl), -(L)ₘ-(C₄-C₁₂ cycloakenyl), —(C₃-C₅ cycloalkylene)ₘ-(C₂-C₁₂ alkynyl), -(L)ₘ-(3-12 member heterocyclyl), -(L)ₘ-(phenyl), -(L)ₘ-(naphthyl), and -(L)ₘ-(5-12 member heteroaryl), wherein each R^{a}, R^{b}, R^{c} and R^{d} is independently optionally further substituted by 1-6 R^{f}; R^{a} and R^{b}, or R^{c} and R^{d}, together with the atom to which they are attached, may optionally form a ring selected from 3-12 member heterocyclyl and 5-12 member heteroaryl, the said ring is optionally further substituted by 1-6 R^{f}; each R^{f} is independently selected from oxo, —(C₁-C₃ alkylene)ₘ-(C₁-C₆ perfluoalkyl), C₁-C₁₂ alkyl, C₂-C₆ alkenyl, C₂-C₆ alkynyl, —(C₁-C₃ alkylene)ₘ-(C₃-C₇ cycloalkyl), —(C₁-C₃ alkylene)ₘ-(3-7 member heterocyclyl), —(C₁-C₃ alkylene)ₘ-(5-7 member heteroaryl), -(L)ₘ-halide, -(L)ₘ-CN, -(L)ₘ-C(O)R^{k}, -(L)ₘ-C(O)OR^{k}, -(L)ₘ-C(O)NR^{k}R^{j}, -(L)ₘ-OR^{k}, -(L)ₘ-OC(O)R^{k}, -(L)ₘ-NO₂, -(L)ₘ-NR^{k}R^{j}, -(L)ₘ-N(R^{k})C(O)₁₂³, -(L)ₘ-O-L-NR^{k}R^{j}, -(L)ₘ-SR^{k}, -(L)ₘ-S(O)R^{k}, -(L)ₘ-S(O)₂R^{j}R^{k}, wherein each R^{f} is independently optionally further substituted by 1-3 groups selected from C₁-C₃ alkyl, halide and C₁-C₃ perfluoroalkyl; each R^{k} and R^{j} is independently —H, —OH, C₁-C₃ perfluoroalkyl, C₁-C₆ alkyl, C₂-C₆ alkenyl, C₃-C₆ alkynyl, —(C₁-C₃ alkylene)ₘ-(C₃-C₆ cycloalkyl) or —(C₁-C₃ alkylene)ₘ-(3 to 6 member heterocyclyl), R^{k} and R^{j} may optionally form a ring selected from 3-7 member heterocyclyl and 5-7 member heteroaryl, with said ring optionally further substituted by 1 to 2 groups selected from C₁-C₃ alkyl, C₁-C₃ perfluoroalkyl, C₁-C₃ alkoxy, oxo, —(C₁-C₃ alkylene)ₘ-halide, —(C₁-C₃ alkylene)ₘ-CN, —(C₁-C₃ alkylene)ₘ-OH, —(C₁-C₃ alkylene)ₘ-NH₂, —(C₁-C₃ alkylene)ₘ-(C₁-C₆ monoalkylamino) and —(C₁-C₃ alkylene)ₘ-(C₂-C₈ dialkylamino); each L is independently a bivalent radical selected from —(C₁-C₆ alkylene)-, —(C₃-C₇ cycloalkylene)-, —(C₁-C₆ alkylene)-(C₃-C₇ cycloalkylene)- and —(C₃-C₇ cycloalkylene)-(C₁-C₆ alkylene)-; each m is independently 0 or 1; and n is 1, 2, or 3.

In certain embodiments, R¹ is 9 or 10-membered bicyclic heteroaryl (e.g., 9-membered bicyclic heteroaryl) optionally substituted with 1, 2, or 3 independent occurrences of C₁-C₆ alkyl (e.g., 1 occurrence of —CH₃). In certain embodiments, R² and R³ are each independently selected from C₁-C₆ alkyl (e.g., both R² and R³ are —CH₃). In certain embodiments, R⁴ is —C(O)NR^{a}R^{b}. In certain embodiments, R^{a} is —H and R^{b} is -(L)ₘ-(phenyl). In certain embodiments, L is C₁-C₆ alkylene substituted with —NR^{k}R^{j} and m is 1. In certain embodiments, R^{k} and R^{j} are each independently selected from C₁-C₆ alkyl (e.g., both R^{k} and R^{j} are —CH₃). In certain embodiments, R¹ is 9 or 10-membered bicyclic heteroaryl (e.g., 9-membered bicyclic heteroaryl) optionally substituted with 1, 2, or 3 independent occurrences of C₁-C₆ alkyl (e.g., 1 occurrence of —CH₃), R² and R³ are each independently selected from C₁-C₆ alkyl (e.g., both R² and R³ are —CH₃), R⁴ is —C(O)NR^{a}R^{b}, R^{a} is —H and R^{b} is -(L)ₘ-(phenyl), L is C₁-C₆ alkylene substituted with —NR^{k}R^{j} and m is 1, and R^{k} and R^{j} are each independently selected from C₁-C₆ alkyl (e.g., both R^{k} and R^{j} are —CH₃).

In some aspects, the PAK4 inhibitor is a compound of Formula (ii)

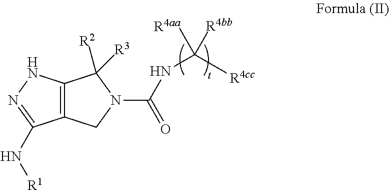

or a pharmaceutically acceptable salt thereof, wherein R¹ is 9 or 10-membered bicyclic heteroaryl optionally substituted with 1, 2, or 3 independent occurrences of C₁-C₆ alkyl (e.g., —CH₃); R² and R³ are each independently selected from C₁-C₆ alkyl (e.g., both R² and R³ are —CH₃); R^{4aa} and R^{4bb} are each independently selected from the group consisting of —H, phenyl, naphthyl, and C₁-C₆ aralkyl; R^{4cc} is —NR^{aa}R^{bb}; R^{aa} and R^{bb} are each independently selected from the group consisting of —H, C₁-C₆ alkyl (e.g., —CH₃), C₂-C₆ alkenyl, C₂-C₆ alkynyl, C₃-C₁₂ cycloalkyl, C₄-C₁₂ cycloalkenyl, 3-12 membered heterocyclyl, and C₁-C₆ aralkyl; and t is an integer selected from the group consisting of 1, 2, and 3. In certain embodiments, R^{aa} and R^{bb} are each independently selected from the group consisting of —H and C₁-C₆ alkyl (e.g., —CH₃).

In certain embodiments, R¹ is

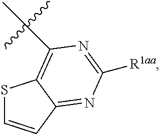

wherein R^{1aa} is C₁-C₆ alkyl (e.g., —CH₃).

LCH-7749944 is an exemplary PAK4 inhibitor. Zhang J, Wang J, Guo Q, Wang Y, Zhou Y, Peng H, Cheng M, Zhao D, Li F (April 2012). “LCH-7749944, a novel and potent p21-activated kinase 4 inhibitor, suppresses proliferation and invasion in human gastric cancer cells”. Cancer Letters. 317 (1): 24-32.

Glaucarubinone is an exemplary PAK4 inhibitor. Yeo D, Huynh N, Beutler J A, Christophi C, Shulkes A, Baldwin G S, Nikfarjam M, He H (May 2014). “Glaucarubinone and gemcitabine synergistically reduce pancreatic cancer growth via down-regulation of P21-activated kinases”. Cancer Letters. 346 (2): 264-72.

KY-04031 is an exemplary PAK4 inhibitor. Ryu B J, Kim S, Min B, Kim K Y, Lee J S, Park W J, Lee H, Kim S H, Park S (July 2014). “Discovery and the structural basis of a novel p21-activated kinase 4 inhibitor”. Cancer Letters. 349 (1): 45-50.

KY-04045 is an exemplary PAK4 inhibitor. Park J K, Kim S, Han Y J, Kim S H, Kang N S, Lee H, Park S (June 2016). “The discovery and the structural basis of an imidazo[4,5-b]pyridine-based p21-activated kinase 4 inhibitor”. Bioorganic & Medicinal Chemistry Letters. 26 (11): 2580-3.

1-phenanthryl-tetrahydroisoquinoline derivatives are exemplary PAK4 inhibitor(s). Song S, Li X, Guo J, Hao C, Feng Y, Guo B, Liu T, Zhang Q, Zhang Z, Li R, Wang J, Lin B, Li F, Zhao D, Cheng M (March 2015). “Design, synthesis and biological evaluation of 1-phenanthryl-tetrahydroisoquinoline derivatives as novel p21-activated kinase 4 (PAK4) inhibitors”. Organic & Biomolecular Chemistry. 13 (12): 3803-18.

(−)-β-hydrastine is an exemplary PAK4 inhibitor. Guo B, Li X, Song S, Chen M, Cheng M, Zhao D, Li F (April 2016). “(−)-(3-hydrastine suppresses the proliferation and invasion of human lung adenocarcinoma cells by inhibiting PAK4 kinase activity”. Oncology Reports. 35 (4): 2246-56.

Inka1 is an exemplary PAK4 inhibitor. Baskaran Y, Ang K C, Anekal P V, Chan W L, Grimes J M, Manser E, Robinson R C (November 2015). “An in cellulo-derived structure of PAK4 in complex with its inhibitor Inka1”. Nature Communications. 6: 8681.

GL-1196 is an exemplary PAK4 inhibitor. Zhang J, Zhang H Y, Wang J, You L H, Zhou R Z, Zhao D M, Cheng M S, Li F (April 2016). “GL-1196 Suppresses the Proliferation and Invasion of Gastric Cancer Cells via Targeting PAK4 and Inhibiting PAK4-Mediated Signaling Pathways”. International Journal of Molecular Sciences. 17 (4): 470.

GNE-2861 is an exemplary PAK4 inhibitor. Zhuang T, Zhu J, Li Z, Lorent J, Zhao C, Dahlman-Wright K, Stromblad S (December 2015). “p21-activated kinase group II small compound inhibitor GNE-2861 perturbs estrogen receptor alpha signaling and restores tamoxifen-sensitivity in breast cancer cells”. Oncotarget. 6 (41): 43853-68.

miR-145 is an exemplary PAK4 inhibitor. Wang Z, Zhang X, Yang Z, Du H, Wu Z, Gong J, Yan J, Zheng Q (October 2012). “MiR-145 regulates PAK4 via the MAPK pathway and exhibits an antitumor effect in human colon cells”. Biochemical and Biophysical Research Communications. 427 (3): 444-9.

miR-433 is an exemplary PAK4 inhibitor. Xue J, Chen L Z, Li Z Z, Hu Y Y, Yan S P, Liu L Y (January 2015). “MicroRNA-433 inhibits cell proliferation in hepatocellular carcinoma by targeting p21 activated kinase (PAK4)”. Molecular and Cellular Biochemistry. 399 (1-2): 77-86.

miR-126 is an exemplary PAK4 inhibitor. Luo P, Fei J, Zhou J, Zhang W (May 2015). “microRNA-126 suppresses PAK4 expression in ovarian cancer SKOV3 cells”. Oncology Letters. 9 (5): 2225-2229.

A PAK4 inhibitor can be an inhibitor that causes a genetic alteration of PAK4, e.g., in cancer. The alteration can be, e.g., a genetic deletion or disruption. An alteration can be a CRISPR-Cas9-induced genetic alteration.

A PAK4 inhibitor can be a CRISPR-Cas9, a TALEN, a meganuclease, or a zinc-finger nuclease. A PAK4 inhibitor can be CRISPR-Cas9. For example, a CRISPR-Cas9 system can include PAK4-targeting sgRNAs. sgRNAs can comprise a forward sgRNA having the sequence of 5′-TTCGAGCACCGTGTACACAC-3′ and a reverse sgRNA having the sequence of 5′-GTGTGTACACGGTGCTCGAA -3′.

A PAK4 inhibitor can be an RNA interference (RNAi) compound. For example, a PAK4 RNAi compound can be small interfering RNA (siRNA), which are known in the art. For example, as disclosed in Paliouras et al., “Pak4, a Novel Gab1 Binding Partner, Modulates Cell Migration and Invasion by the Met Receptor”, Molecular and Cellular Biology 2009 June; 29(11):3018-32. doi: 10.1128/MCB.01286-08. Epub 2009 Mar. 16. (see materials and methods section at: duplex 1, CCGGCTGGTGGCCGTCAAGAA; duplex 4, CGAGAACGTGGTGGAGATGTA).

A PAK4 inhibitor can be an inhibitor of a microRNA, optionally wherein the microRNA is at least one of miR-145, miR-433, and miR-126

### Additional Agents

In some embodiments, a PAK4 inhibitor provided herein is administered with at least one additional therapeutic agent. Any suitable additional therapeutic agent may be administered with a PAK4 inhibitor provided herein. In some aspects, the additional therapeutic agent is selected from radiation, a cytotoxic agent, a chemotherapeutic agent, a cytostatic agent, an anti-hormonal agent, an immunostimulatory agent, an anti-angiogenic agent, and combinations thereof. An additional agent can be chemotherapy. An additional agent can be radiotherapy. An additional agent can be hormone inhibitor therapy.

In some embodiments, the additional therapeutic agent comprises an immunostimulatory agent. An exemplary immunostimulatory agent includes a checkpoint inhibitor such as an anti-PD1 antibody.

In some embodiments, the immunostimulatory agent is an agent that blocks signaling of an inhibitory receptor of an immune cell, or a ligand thereof. In some aspects, the inhibitory receptor or ligand is selected from CTLA-4, PD-1, PD-L1, LAG-3, Tim3, TIGIT, neuritin, BTLA, KIR, and combinations thereof. In some aspects, the agent is selected from an anti-PD1 antibody (e.g., pembrolizumab or nivolumab), and anti-PD-L1 antibody (e.g., atezolizumab), an anti-CTLA-4 antibody (e.g., ipilimumab), and combinations thereof.

In some embodiments, the immunostimulatory agent is an agonist of a co-stimulatory receptor of an immune cell. In some aspects, the co-stimulatory receptor is selected from OX40, ICOS, CD27, CD28, 4-1BB, or CD40. In some embodiments, the agonist is an antibody.

In some embodiments, the immunostimulatory agent is a cytokine. In some aspects, the cytokine is selected from IL-2, IL-5, IL-7, IL-12, IL-15, IL-21, and combinations thereof.

An immunostimulatory agent can be a checkpoint inhibitor. An immunostimulatory agent can be a PD1 inhibitor. An immunostimulatory agent can be a PDL1 inhibitor. An immunostimulatory agent can be a CTLA4 inhibitor.

An immunostimulatory agent can be a LAG3 inhibitor. An immunostimulatory agent can be a TIN/13 inhibitor. An immunostimulatory agent can be a TIGIT inhibitor. An immunostimulatory agent can be a CSF1R inhibitor. An immunostimulatory agent can be a PEGylated cytokine (such as at least one of IL-2, IL-10, or IFN). An immunostimulatory agent can be a GITR antibody. An immunostimulatory agent can be an A2AR inhibitor. An immunostimulatory agent can be an IDO inhibitor. An immunostimulatory agent can be an antibody to at least one of GITR, OX40, CD40, or CD137/41BB.

An immunostimulatory agent can be a checkpoint inhibitor.

An immunostimulatory agent can be an anti-PD1 antibody.

An immunostimulatory agent can be an anti-PDL1 antibody.

An immunostimulatory agent can be an anti-CTLA4 antibody.

An immunostimulatory agent can be pembrolizumab (Keytruda). An immunostimulatory agent can be nivolumab (Opdivo). An immunostimulatory agent can be atezolizumab (Tecentriq). An immunostimulatory agent can be avelumab (Bavencio). An immunostimulatory agent can be durvalumab (Imfinzi). An immunostimulatory agent can be BMS-936559/MDX1105. An immunostimulatory agent can be PDR001/spartalizumab. An immunostimulatory agent can be GLS-010/AB-122. An immunostimulatory agent can be PF-06801591. An immunostimulatory agent can be BGB-a317. An immunostimulatory agent can be INCSHR-1210. An immunostimulatory agent can be TSR-042. An immunostimulatory agent can be JS-001. An immunostimulatory agent can be LY3300054. An immunostimulatory agent can be ipilimumab (Yervoy). An immunostimulatory agent can be tremelimumab. An immunostimulatory agent can be AGEN-1884.

In some embodiments, the immunostimulatory agent is an oncolytic virus. In some aspects, the oncolytic virus is selected from a herpes simplex virus, a vesicular stomatitis virus, an adenovirus, a Newcastle disease virus, a vaccinia virus, and a maraba virus.

An immunostimulatory agent can be an oncolytic virus. An immunostimulatory agent can be a TLR agonist. An immunostimulatory agent can be a STING agonist. An immunostimulatory agent can be a RIG-I agonist. An immunostimulatory agent can be a MDA5 agonist.

In some embodiments, the immunostimulatory agent is a T cell with a chimeric antigen receptor (CAR-T cell). In some embodiments, the immunostimulatory agent is a bi- or multi-specific T cell directed antibody. In some embodiments, the immunostimulatory agent is an anti-TGF-β antibody. In some embodiments, the immunostimulatory agent is a TGF-β trap.

An immunostimulatory agent can be one or more T cells modified to express a chimeric antigen receptor (CAR).

An immunostimulatory agent can be one or more T cells modified to express a transgenic T cell receptor (TCR).

An immunostimulatory agent can be one or more tumor-infiltrating lymphocytes (TILs).

In some embodiments, the additional therapeutic agent is a vaccine to a tumor antigen. Any suitable antigen may be targeted by the vaccine, provided that it is present in a tumor treated by the methods provided herein. In some aspects, the tumor antigen is a tumor antigen that is overexpressed in comparison its expression levels in normal tissue. In some aspects, the tumor antigen is selected from cancer testis antigen, differentiation antigen, NY-ESO-1, MAGE-A1, MART, and combinations thereof.

Further examples of additional therapeutic agents include a taxane (e.g., paclitaxel or docetaxel); a platinum agent (e.g., carboplatin, oxaliplatin, and/or cisplatin); a topoisomerase inhibitor (e.g., irinotecan, topotecan, etoposide, and/or mitoxantrone); folinic acid (e.g., Leucovorin); or a nucleoside metabolic inhibitor (e.g., fluorouracil, capecitabine, and/or gemcitabine). In some embodiments, the additional therapeutic agent is folinic acid, 5-fluorouracil, and/or oxaliplatin. In some embodiments, the additional therapeutic agent is 5-fluorouracil and irinotecan. In some embodiments, the additional therapeutic agent is a taxane and a platinum agent. In some embodiments, the additional therapeutic agent is paclitaxel and carboplatin. In some embodiments, the additional therapeutic agent is pemetrexate. In some embodiments, the additional therapeutic agent is a targeted therapeutic such as an EGFR-, RAF- or MEK-targeted agent.

The additional therapeutic agent may be administered by any suitable means. In some embodiments, a PAK4 inhibitor provided herein and the additional therapeutic agent are included in the same pharmaceutical composition. In some embodiments, a PAK4 inhibitor provided herein and the additional therapeutic agent are included in different pharmaceutical compositions.

In embodiments where a PAK4 inhibitor provided herein and the additional therapeutic agent are included in different pharmaceutical compositions, administration of a PAK4 inhibitor can occur prior to, simultaneously, and/or following, administration of the additional therapeutic agent. In some aspects, administration of a PAK4 inhibitor provided herein and the additional therapeutic agent occur within about one month of each other. In some aspects, administration of a PAK4 inhibitor provided herein and the additional therapeutic agent occur within about one week of each other. In some aspects, administration of a PAK4 inhibitor provided herein and the additional therapeutic agent occur within about one day of each other. In some aspects, administration of a PAK4 inhibitor provided herein and the additional therapeutic agent occur within about twelve hours of each other. In some aspects, administration of a PAK4 inhibitor provided herein and the additional therapeutic agent occur within about one hour of each other.

PAK4 Inhibitor Uses and Cancer Treatment

For therapeutic applications, a PAK4 inhibitor is administered to a mammal, generally a human, in a pharmaceutically acceptable dosage form such as those known in the art and those discussed above. For example, the PAK4 inhibitor may be administered to a human intravenously as a bolus or by continuous infusion over a period of time, by oral, intramuscular, intraperitoneal, intra-cerebrospinal, subcutaneous, intra-articular, intrasynovial, intrathecal, or intratumoral routes. The PAK4 inhibitor can also be suitably administered by peritumoral, intralesional, or perilesional routes, to exert local as well as systemic therapeutic effects.

PAK4 inhibitors provided herein may be useful for the treatment of any disease or condition involving PAK4. In some embodiments, the disease or condition is a disease or condition that can benefit from treatment with a PAK4 inhibitor. In some embodiments, the disease or condition is a tumor. In some embodiments, the disease or condition is a cell proliferative disorder. In some embodiments, the disease or condition is a cancer.

In some embodiments, the PAK4 inhibitors provided herein are provided for use as a medicament. In some embodiments, the PAK4 inhibitors provided herein are provided for use in the manufacture or preparation of a medicament. In some embodiments, the medicament is for the treatment of a disease or condition that can benefit from a PAK4 inhibitor. In some embodiments, the disease or condition is a tumor. In some embodiments, the disease or condition is a cell proliferative disorder.

In some embodiments, provided herein are methods of treating a disease or condition in a subject in need thereof by administering an effective amount of a PAK4 inhibitor provided herein to the subject. In some aspects, the disease or condition is a cancer.

Any suitable cancer may be treated with the PAK4 inhibitors provided herein. Illustrative suitable cancers include, for example, acute lymphoblastic leukemia (ALL), acute myeloid leukemia (AML), adrenocortical carcinoma, anal cancer, appendix cancer, astrocytoma, basal cell carcinoma, brain tumor, bile duct cancer, bladder cancer, bone cancer, breast cancer, bronchial tumor, carcinoma of unknown primary origin, cardiac tumor, cervical cancer, chordoma, colon cancer, colorectal cancer, craniopharyngioma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, lip and oral cavity cancer, liver cancer, lobular carcinoma in situ, lung cancer, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and par nasal sinus cancer, nasopharyngeal cancer, neuroblastoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, and Wilms tumor.

A cancer can be PAK4 positive (+). A cancer can be resistant to treatment with a checkpoint inhibitor alone. A cancer can be resistant to treatment with an immunostimulatory agent alone.

A cancer can be cutaneous melanoma, microsatellite unstable cancers of any histology, head and neck carcinoma, lung carcinoma, renal cell carcinoma, bladder cancer, Merkel cell carcinoma, Hodgkin's lymphoma, gastroesophageal carcinoma, or hepatocellular carcinoma that are resistant to a prior therapy with anti-PD-1, anti-PD-L1, or anti-CTLA4 antibody therapy.

A cancer can be a cancer known to have a low likelihood of responding to treatment with a checkpoint inhibitor alone, optionally wherein the cancer is pancreatic cancer, colorectal cancer, breast cancer, prostate cancer, adrenocortical carcinoma, testicular and germinal cell tumors, glioblastoma multiforme, uveal melanoma, thyroid cancer, endometrial cancer, ovarian cancer, cervical carcinoma, cholangiocarcinoma, mesothelioma, thymoma, a lymphoma, a leukemia, multiple myeloma, or a sarcoma.

A cancer can be pancreatic cancer, colorectal cancer, breast cancer, adrenocortical carcinoma, testicular and germinal cell tumors, glioblastoma multiforme, uveal melanoma, thyroid cancer, endometrial cancer, ovarian cancer, cervical carcinoma, cholangiocarcinoma, mesothelioma, thymoma, a lymphoma, a leukemia, multiple myeloma or a sarcoma, with the PAK4 inhibitor given together with an immune checkpoint inhibitor along with standard of care chemotherapy and/or radiotherapy.

A cancer can be estrogen/progesterone receptor positive breast cancer, or prostate cancer, with the PAK4 inhibitor given together with an immune checkpoint inhibitor and hormone inhibitor therapy.

A cancer can be uveal melanoma, with the PAK4 inhibitor given together with an immune checkpoint inhibitor and one or more immune modulators such as a LAG3 inhibitor, a TIM3 inhibitor, a TIGIT inhibitor, a CSF1R inhibitor, a PEGylated cytokine (optionally IL-2, IL-10, IFN), a GITR antibody, a A2AR inhibitor, an IDO inhibitor, or an antibody to OX40, CD40, or CD137/41BB.

A cancer can be pancreatic cancer, colorectal cancer, breast cancer, adrenocortical carcinoma, testicular and germinal cell tumors, glioblastoma multiforme, uveal melanoma, thyroid cancer, endometrial cancer, ovarian cancer, cervical carcinoma, cholangiocarcinoma, mesothelioma, thymoma, a lymphoma, a leukemia, multiple myeloma or a sarcoma, with the PAK4 inhibitor given together with an immune checkpoint inhibitor and one or more immune modulators such as a LAG3 inhibitor, a TIM3 inhibitor, a TIGIT inhibitor, a CSF1R inhibitor, a PEGylated cytokine (optionally IL-2, IL-10, IFN), a GITR antibody, a A2AR inhibitor, an IDO inhibitor, or an antibody to OX40, CD40, or CD137/41BB.

A cancer can be cutaneous melanoma, microsatellite unstable cancers of any histology, head and neck carcinoma, lung carcinoma, renal cell carcinoma, bladder cancer, Merkel cell carcinoma, Hodgkin's lymphoma, gastroesophageal carcinoma, or hepatocellular carcinoma, with the PAK4 inhibitor given together with an immune checkpoint inhibitor and one or more immune modulators such as a LAG3 inhibitor, a TIM3 inhibitor, a TIGIT inhibitor, a CSF1R inhibitor, a PEGylated cytokine (optionally IL-2, IL-10, IFN), a GITR antibody, a A2AR inhibitor, an IDO inhibitor, an antibody to OX40, CD40, or CD137/41BB.

A cancer can be pancreatic cancer, colorectal cancer, breast cancer, adrenocortical carcinoma, testicular and germinal cell tumors, glioblastoma multiforme, uveal melanoma, thyroid cancer, endometrial cancer, ovarian cancer, cervical carcinoma, cholangiocarcinoma, mesothelioma, thymoma, a lymphoma, multiple myeloma or a sarcoma, with the PAK4 inhibitor given together with an immune checkpoint inhibitor and intratumoral injection of one or more immune stimulating agents such as an oncolytic virus, a TLR agonist, a STING agonist, a RIG-I agonist, or a MDA5 agonist.

A cancer can be cutaneous melanoma, microsatellite unstable cancers of any histology, head and neck carcinoma, lung carcinoma, renal cell carcinoma, bladder cancer, Merkel cell carcinoma, Hodgkin's lymphoma, gastroesophageal carcinoma, or hepatocellular carcinoma, with the PAK4 inhibitor given together with an immune checkpoint inhibitor and intratumoral injection of one or more immune stimulating agents such as an oncolytic virus, a TLR agonist, a STING agonist, a RIG-I agonist, or an MDA5 agonist.

A cancer can be a lymphoma, a leukemia or multiple myeloma with the PAK4 inhibitor given together with the adoptive cell transfer of T cells modified to express a chimeric antigen receptor (CAR).

A cancer can be a solid tumor with the PAK4 inhibitor given together with the adoptive cell transfer of T cells modified to express a transgenic T cell receptor (TCR).

A cancer can be a solid tumor with the PAK4 inhibitor given together with the adoptive cell transfer of tumor-infiltrating lymphocytes (TILs).

In some embodiments, provided herein is a method of inhibiting PAK4 in a target cell of a subject in need thereof by administering an effective amount of a PAK4 inhibitor provided herein to the subject.

In some embodiments, provided herein is a method of increasing the proliferation, survival, and/or function of an effector T cell in a subject in need thereof by administering an effective amount of a PAK4 inhibitor provided herein to the subject. In some aspects the effector T cell is a CD4+ effector T cell. In some aspects, the effector T cell is a CD8+ effector T cell.

In some embodiments, provided herein is a method of increasing the activity of a natural killer (NK) or natural killer T (NKT) cell in a subject in need thereof by administering an effective amount of a PAK4 inhibitor provided herein to the subject.

In some embodiments, provided herein is a method of enhancing an immune response in a subject in need thereof by administering an effective amount of a PAK4 inhibitor provided herein to the subject.

In some embodiments, provided herein is a method delaying the onset of a tumor in a subject in need thereof by administering an effective amount of a PAK4 inhibitor provided herein to the subject.

In some embodiments, provided herein is a method preventing the onset of a tumor in a subject in need thereof by administering an effective amount of a PAK4 inhibitor provided herein to the subject.

In some embodiments, provided herein is a method of delaying the onset of a cancer in a subject in need thereof by administering an effective amount of a PAK4 inhibitor provided herein to the subject.

In some embodiments, provided herein is a method of preventing the onset of a cancer in a subject in need thereof by administering an effective amount of a PAK4 inhibitor provided herein to the subject.

In some embodiments, provided herein is a method of reducing the size of a tumor in a subject in need thereof by administering an effective amount of a PAK4 inhibitor provided herein to the subject.

In some embodiments, provided herein is a method of reducing the number of metastases in a subject in need thereof by administering an effective amount of a PAK4 inhibitor provided herein to the subject.

### Pharmaceutical Compositions

Methods for treatment of PAK4-related diseases are also encompassed by the present disclosure. The methods can include administering a therapeutically effective amount of a PAK4 inhibitor alone or in combination with an immunostimulatory agent. A PAK4 inhibitor can be formulated in pharmaceutical compositions. These compositions can comprise, in addition to one or more of the PAK4 inhibitors, a pharmaceutically acceptable excipient, carrier, buffer, stabiliser or other materials well known to those skilled in the art. Such materials should be non-toxic and should not interfere with the efficacy of the active ingredient. The precise nature of the carrier or other material can depend on the route of administration, e.g. oral, intravenous, cutaneous or subcutaneous, nasal, intramuscular, intraperitoneal routes.

Pharmaceutical compositions for oral administration can be in tablet, capsule, powder or liquid form. A tablet can include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol can be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives can be included, as required.

A PAK4 inhibitor that is to be given to an individual, administration is preferably in a “therapeutically effective amount” or “prophylactically effective amount” (as the case can be, although prophylaxis can be considered therapy), this being sufficient to show benefit to the individual. The actual amount administered, and rate and time-course of administration, will depend on the nature and severity of protein aggregation disease being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980.

A composition can be administered alone or in combination with other treatments, either simultaneously or sequentially dependent upon the condition to be treated.

### Kits

Also provided are kits comprising one or more PAK4 inhibitors provided herein. The kits may be used for the treatment, prevention, and/or diagnosis of a disease or disorder, as described herein.

In some embodiments, the kit comprises a container and a label or package insert on or associated with the container. Suitable containers include, for example, bottles, vials, syringes, and IV solution bags. The containers may be formed from a variety of materials, such as glass or plastic. The container holds a composition that is by itself, or when combined with another composition, effective for treating, preventing and/or diagnosing a disease or disorder. The container may have a sterile access port. For example, if the container is an intravenous solution bag or a vial, it may have a port that can be pierced by a needle. At least one active agent in the composition is a PAK4 inhibitor provided herein. The label or package insert indicates that the composition is used for treating the selected condition.

In some embodiments, the kit comprises (a) a first container with a first composition contained therein, wherein the first composition comprises a PAK4 inhibitor provided herein; and (b) a second container with a second composition contained therein, wherein the second composition comprises a further therapeutic agent (e.g., an immunostimulatory agent). The kit in this embodiment of the invention may further comprise a package insert indicating that the compositions can be used to treat a particular condition such as cancer.

Alternatively, or additionally, the kit may further comprise a second (or third) container comprising a pharmaceutically-acceptable excipient. In some aspects, the excipient is a buffer. The kit may further include other materials desirable from a commercial and user standpoint, including filters, needles, and syringes.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3^{rd} Ed. (Plenum Press) Vols A and B(1992).

Materials and Methods

Patients, Tumor Biopsies and Response Assessment

Tumor biopsies were collected under UCLA Institutional Review Board approvals 11-001918 and 11-003066 from 41 patients with metastatic melanoma treated with either pembrolizumab or nivolumab after signing a written informed consent. Samples were immediately stored in RNAlater (Ambion, Foster City, Calif.) or snap frozen in liquid nitrogen for subsequent RNA extraction. Response was assessed for each biopsy independently.

RNA Isolation and RNA-Seq Analysis

We obtained a total of 66 tumor samples from which we extracted RNA using the AllPrep DNA/RNA mini kit (Qiagen, Hilden, Germany) and mirVANA miRNA Isolation Kit (Ambion, Foster City, Calif.). Poly-A selection was used for library construction and samples were sequenced using the Illumina HiSeq2500 platform with a read length of 2×100 at the UCLA Technology Center for Genomics & Bioinformatics. Raw FASTQ files were aligned to the hg19 genome using HISAT2 version 2.0.4²⁴ using the default parameters and counted with HTseq version 0.6.1²⁵ with the intersection-nonempty mode and counting ambiguous reads if fully overlapping. Raw counts were then normalized to fragments per kilobase of transcript per million mapped reads (FPKM). Two tumor biopsies were excluded from the analysis due to discordancy with previous immunohistochemistry analysis (data not shown). Four tumor biopsies were excluded based on the expression of KRT15 and KRT5 (data not shown). A total of 60 tumor biopsies were considered for transcriptomic analysis. RNA-seq based cell deconvolution of tissue-infiltrating and stromal population was performed using MCP-counter¹³ using the default settings and immune cell infiltration was defined using the upper and lower quartile scores for each of the obtained immune cell populations. Differential gene expression and principal component analyses were performed using DESeq2 package²⁶. In order to identify enriched signalling pathways, we utilized Gene Set Enrichment Analysis (GSEA) with the following gene sets: C₂ Curated Gene Sets and C₅ Gene Ontology Gene Sets²⁷. Pan-cancer correlation analysis between PAK4 expression and immune cell infiltration (calculated using MCP-counter as described above) was performed using gene expression data from 32 tumor types from TCGA Research Network (http://cancergenome.nih.gov/).

Cell Lines and CRISPRICas9

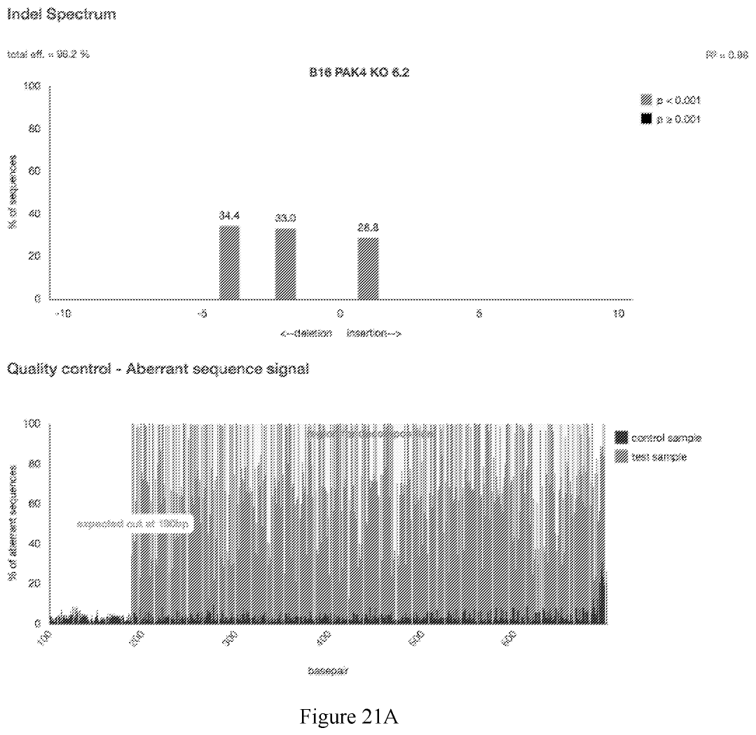

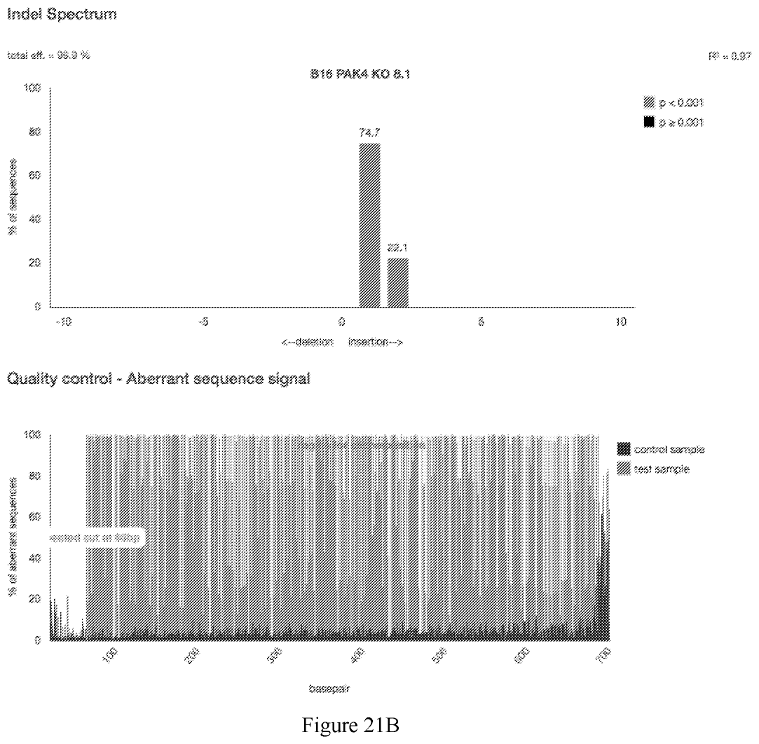

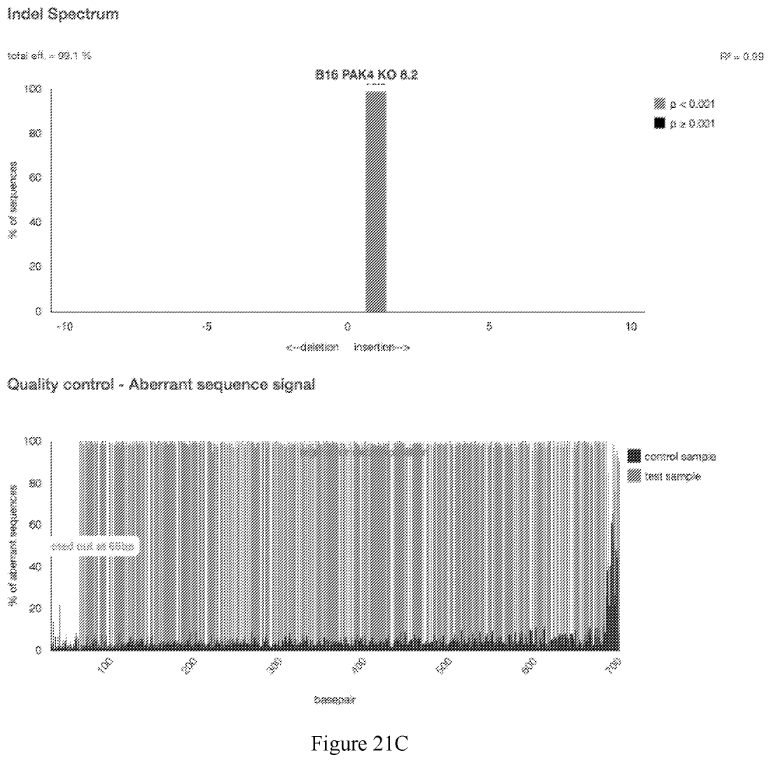

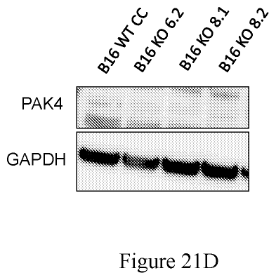

Murine B16 and MC38 cells were maintained in DMEM and RPMI medium respectively, supplemented with 10% FBS, 100 units/mL penicillin, and 100 μg/mL streptomycin at 37° C. in a humidified atmosphere of 5% CO₂. The following sgRNAs targeting PAK4 were used: forward 5-TTCGAGCACCGTGTACACAC-3 and reverse 5-GTGTGTACACGGTGCTCGAA -3 and cloned into the pSpCas9(BB)-2A-GFP vector (Addgene, Cambridge, Mass.) as described in Zheng's protocol²⁸. Mouse cells were then transfected with PAK4-sgRNA plasmid using lipofectamine 3000 (Thermo Fisher Scientific, Waltham, Mass.) and GFP positive cells were collected and single cell sorted 48 hours after transfection at the UCLA Flow Cytometry core. Genomic DNA was isolated for each clone (NucleoSpin Tissue XS, Macherey-Nagel, Duren, Germany) and after PCR amplifying PAK4 sequence, we used Tracking of Indels by Decomposition (TIDE)²⁹ web tool to evaluate and confirm knock out efficiency (FIG. 21). PAK4 deletion was also validated by Western blot, performed as described previously³⁰. Immunoreactivity was assessed with an ECL-Pus Kit (Amersham Biosciences Co., Little Chalfont, UK) and analysed using the ChemiDoc MP system (Bio-rad Laboratories, Hercules, Calif.) (FIG. 21).

Proliferation Assays

Murine melanoma B16 WT and PAK4 KO cells were cultured as described above and supplemented with different concentrations of murine murine TNF-α (R&D systems, Minneapolin, Minn.). Proliferation rates were assessed measuring cell confluence using IncuCyte S3 Live-Cell Analysis System (Essen BioScience, Ann Arbor, Mich.).

Mouse Model Studies

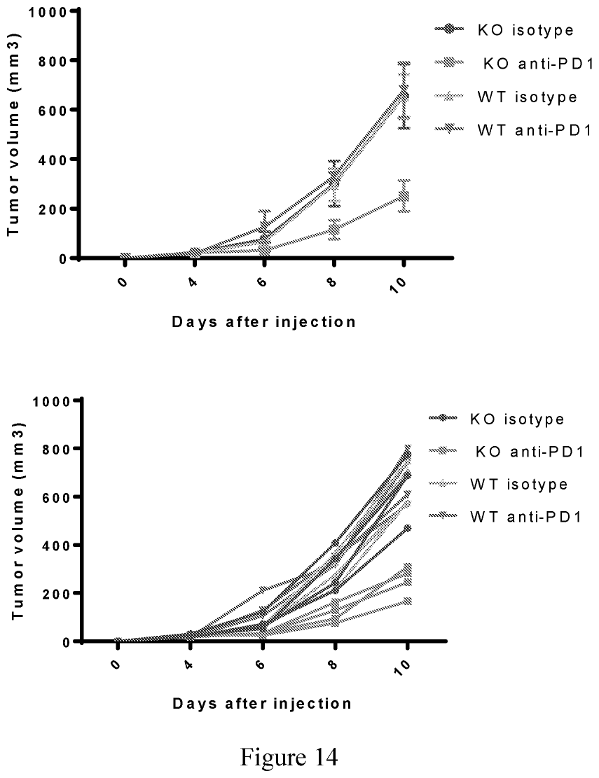

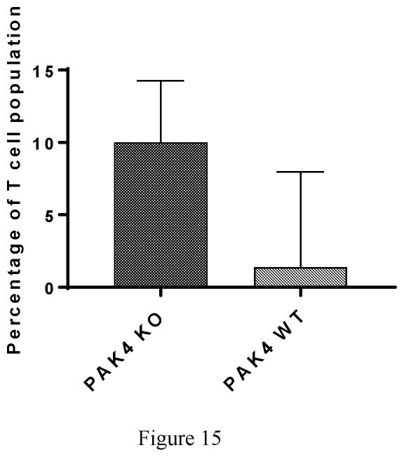

All mouse studies were performed under UCLA Animal Research Committee protocol #2004-159-23. C₅₇BL/6 mice were bred and kept under defined-flora pathogen-free conditions at the Association for Assessment and Accreditation of Laboratory Animal Care approved animal facility of the Division of Experimental Radiation Oncology, UCLA. To study the in vivo effect of PD-1 blockade in anti-tumor response and immune cell infiltration, we subcutaneously (s.c.) injected 0.3×10⁶ B16 PAK4 KO melanoma cells or MC38 PAK4 KO cells into the flanks of C57BL/6 syngeneic mice. 96 hours after tumor injection mice were randomly assigned into the different groups. Anti PD-1 (Cat. No. BE0146, clone RMP1-14, BioXCell, West Lebanon, N.H.) treatment was injected intraperitoneally three times per week at 200 μg per dose. For CD8 depletion studies, we administered anti-CD8 (Cat. No. BE0117, clone YST 169.4, BioXCell) one day before anti-PD1 treatment and then it was co-administered with anti-PD1 for a total of four doses. Splenocytes from control and CD8 depleted mice were taken to validate CD8 depletion efficacy (FIGS. 14-15). To study the combination effects of PAK4 inhibition (KPT-9274) and anti-PD-1 (Cat. No. BE0146, clone RMP1-14, BioXCell) in immune cell infiltration and anti-tumor response, 0.5×10⁶ B16 WT or MC38 WT cells were injected s.c. into the flanks of C57BL/6 mice. 96 hours after tumor injection mice were randomly assigned into the different groups. PAK4 inhibitor was administered twice a day by oral gavage at 150 mg/kg while anti-PD-1 treatment was administered as described above. Tumor progression was monitored three times per week by measuring two perpendicular dimensions with a calliper.

Mass Cytometry

To study the different immune cell populations in the tumor microenvironment of melanoma B16 PAK4 KO and B16 WT tumors, we collected spleen and tumor samples from anti-PD1 treated or untreated mice for each of the two conditions. Tumor samples were processed using the tumor dissociation kit, mouse (Miltenyi, Bergisch Gladbach, Germany) following manufacture's protocol. Spleens were manually disaggregated and filtered with a 70 μm strainer following digestion with the ACK lysis buffer (Lonza, Basel, Switzerland). Samples were then stained and processed as previously described³¹ with two deviations: samples were not barcoded and 3% paraformaldehyde was used instead. Following staining, samples were analysed using the Helios mass cytometer (Fluidigm, South San Francisco, Calif.) platform at the UCLA Flow Cytometry core. Sample quality control was assessed measuring the fluctuation/disruption over time. Calibration beads (Cs140) were also excluded. Samples were pre-gated for cells, singlets and double expression of viable CD45 single cell positive population using FlowJo software (v10.4.2, Ashland, Oreg.) and used as the input for Cytofkit³² which was analysed in R (v3.5.1). To identify and annotate each of the clusters obtained, cluster median data was normalized and a threshold of >0.5 was used to define positive immune markers³¹. t-Distributed Stochastic Neighbor Embedding (t-SNE) plots were generated by PhenoGraph clustering through cytofkiyShinyAPP from Cytofkit.

Immunohistochemistry

We re-analysed IHC samples used in our prior work¹ with matching RNA-seq data to correlate immune cell infiltration between immunohistochemistry and RNA-seq. We generated new slides for two representative patients and stained them with hematoxylin and eosin, S100, CD8, PAK4 and CTNNB1 at the UCLA Anatomic Pathology MC Laboratory. Leica Bond III autostainers (Leica Biosystems, Buffalo Grove, Ill.) were used for immunostaining as previously described¹. Cell density (cells/mm²) was calculated using the Indica Labs Halo¹ (Corrales, N. Mex.).

WNT Activity Assays

β-catenin protein levels and phosphorylation were investigated by Western Blot using the following antibodies: β-catenin (Cat. No. 9587), phospho-β-catenin (S675) (Cat. No. 9567) and phospho-β-catenin (S33/37/T41) (Cat. No. 9561), from Cell Signaling Technology, Danvers, Mass. Cytoplasm and nuclear extraction were performed with NE-PER™Nuclear and Cytoplasmic Extraction Reagents (Thermo Fisher Scientific, Waltham, Mass.) following manufacture's protocol.

For Topflash WNT activity assay cells were plated in 24 well plates and were co-transfected with pSV-β-galactosidase control vector (PR-E1081, Promega, Madison, Wis.) along with either pTopflash (Addgene, Cat. No. 12456) or pFopflash (Addgene, Cat. No. 12457). 24 hours after transfection, cells were treated with Wnt-3a (R&D Systems, Minneapolins, Minn.) at 200 ng/mL. After 8 hours, cells were harvested using Reporter Lysis Buffer (Promega, Cat. No. PR-E4030) and luciferase activity was measured using Bright-Glo Luciferase Assay System (Promega, Cat. No. PR-E2610) and Beta-Glo Assay System (Promega, Cat. No. PR-E4720). Luciferase activity was normalized to its corresponding Beta-Glo activity to account for transfection efficiency.

Tyrosinase expression was measured by qPCR following manufacturer's protocol for the Power SYBR® Green RNA-to-CT™ 1-Step Kit (Applied Biosystems, Foster City, Calif.) and using the primers: 5′ GCACCTATCGGCCATAACAG 3′ and 5′ GCCAGATACGACTGGCTTGT 3′.

### Example 1: PAK4 Expression is Anti-Correlated with Immune Infiltration Across Multiple Cancer Types

We sought to determine tumor-intrinsic drivers of T cell exclusion by comparing tumor biopsies with signatures of immune response. Differential gene expression analysis revealed 591 genes enriched in the group without immune infiltrate gene expression when analysing samples based on dendritic cell infiltration (log 2FC>1, q<0.05, data not shown).

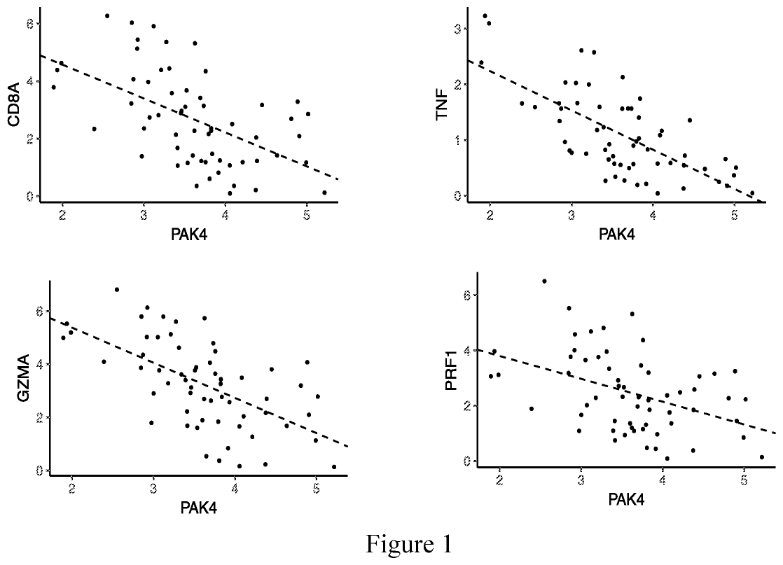

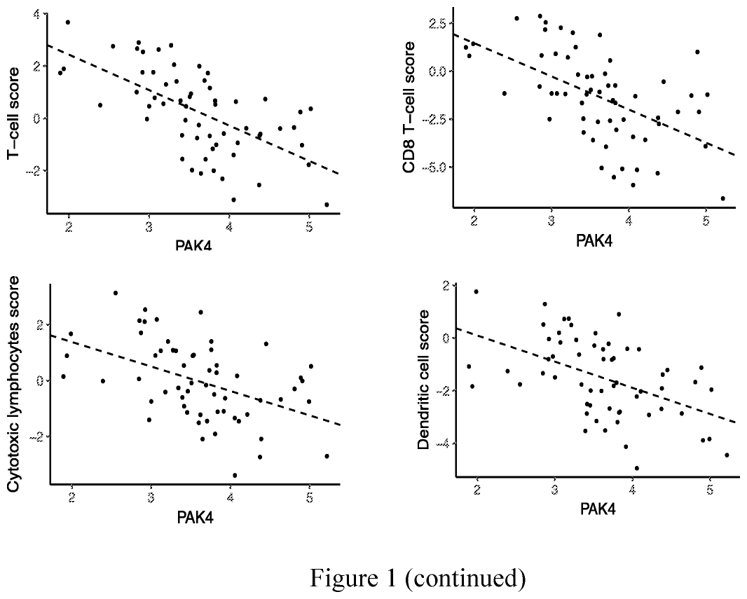

P21 (RAC1) Activated Kinase 4 (PAK4) gene expression was consistently higher in tumor biopsies with low T cell (q<0.0001) and dendritic cell (q<0.0001, data not shown) infiltration, and was also validated using a previously published cohort of 99 biopsies analysed by RNA-seq⁴ (data not shown). PAK4 is a serine/threonine kinase that functions downstream the small GTPases CDC42 and RAC and plays an important role in several signalling pathways involved in tumorigenesis^{6,14}. Previous work from Spranger et al. demonstrated that tumor-intrinsic β-catenin signalling could impair T cell infiltration in melanoma¹⁵. PAK4 phosphorylates and shuttles β-catenin to the nucleus to activate WNT/β-catenin pathway^{7,8,16}. Concordantly, we found that tumor biopsies with high PAK4 expression had increased levels of CTNNB1 and MYC compared to low PAK4-expressing tumor biopsies (data not shown). PAK4 high tumors were also enriched for and positively correlated with a previously reported WNT signature¹⁷ (data not shown). Furthermore, PAK4 negatively correlated with immune markers of an active CD8 T cell response including CD8A, TNF, GZMA and PRF1, as well as with transcriptome signature of different immune cell populations: T cells, CD8 T cells, cytotoxic T cells and dendritic cells (FIG. 1). To determine if PAK4 was expressed by melanoma cancer cells we performed immunohistochemistry analysis of on treatment tumor biopsies. PAK4 co-localized with the melanoma marker S100 (data not shown). In addition, IHC analysis also showed that β-catenin co-localized with PAK4 and validated the inverse correlation between PAK4 and CD8 T cell infiltration observed by RNA-seq (data not shown).

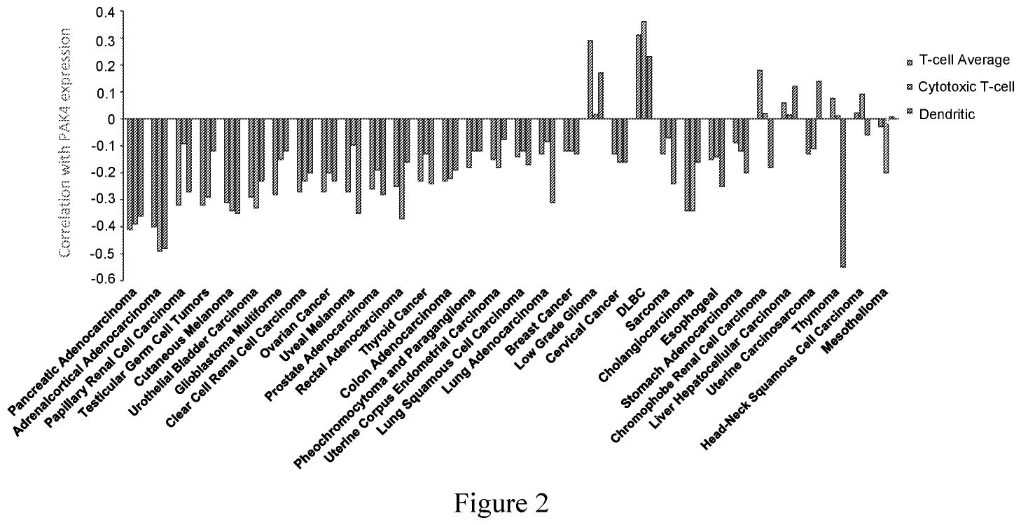

We then investigated whether the association between PAK4 expression and the lack of T cell infiltration in melanoma tumor biopsies could be expanded to other tumor types. To do so, we analysed TCGA transcriptome data from 32 different cancer types and calculated the correlation between PAK4 expression and T cell, cytotoxic lymphocytes and dendritic cell scores generated using MCP-counter¹³ in all of the samples for each cancer type. In addition to cutaneous melanoma, we observed a negative correlation with T cell infiltration in the majority of cancer types, including cancers that are notoriously resistant to anti-PD-1 therapy including pancreatic adenocarcinoma, adrenocortical carcinoma, germ cell cancers, glioblastoma multiforme and prostate cancer (FIG. 2). Additional, relevant cancer types are shown in FIG. 2.

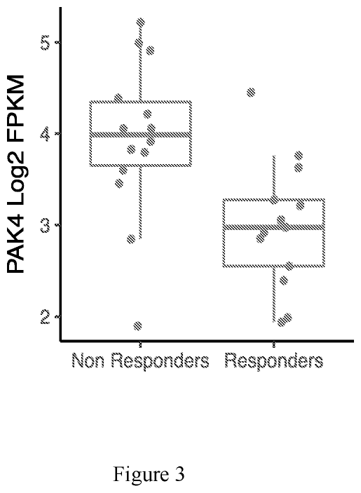

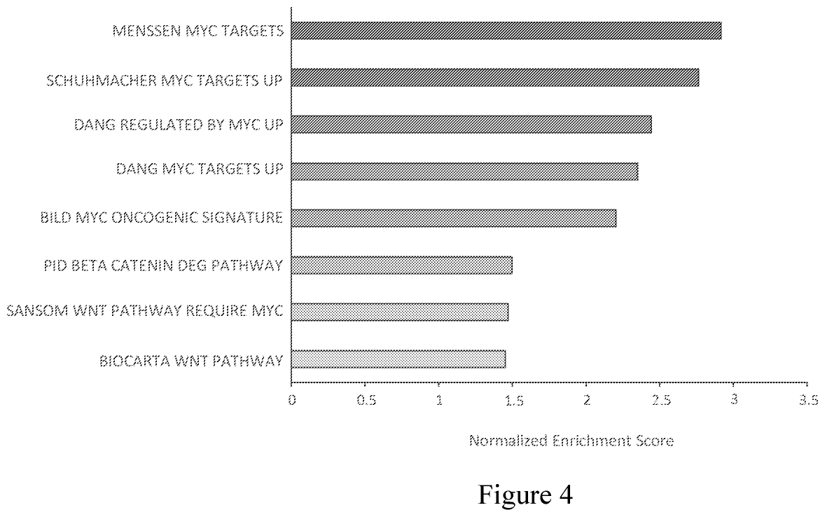

As PAK4 showed a strong inverse correlation with both dendritic cells and T cells in melanoma, we reasoned that tumor biopsies from patients without a response to anti-PD-1 may have an enriched PAK4 expression. Expression of PAK4 transcripts were significantly higher in non-responding biopsies (P=0.004, FIG. 3). We also investigated whether our cohort of non-responding tumor biopsies to PD-1 blockade recapitulated known oncogenic mechanism of T cell exclusion⁵. To test this hypothesis, we compared on-treatment non-responding biopsies to on-treatment responding biopsies and applied GSEA using the curated gene sets. Signatures enriched in on-treatment non-responding biopsies included gene sets related to WNT/β-catenin signalling and the WNT target gene MYC pathways (FIG. 4). In sum, biopsies from patients without a response to PD-1 blockade are enriched for PAK4 expression and gene signatures related to known oncogenic pathways involved in T cell exclusion⁵.

### Example 2: PAK4 Inhibition to Treat Cancer In Vivo

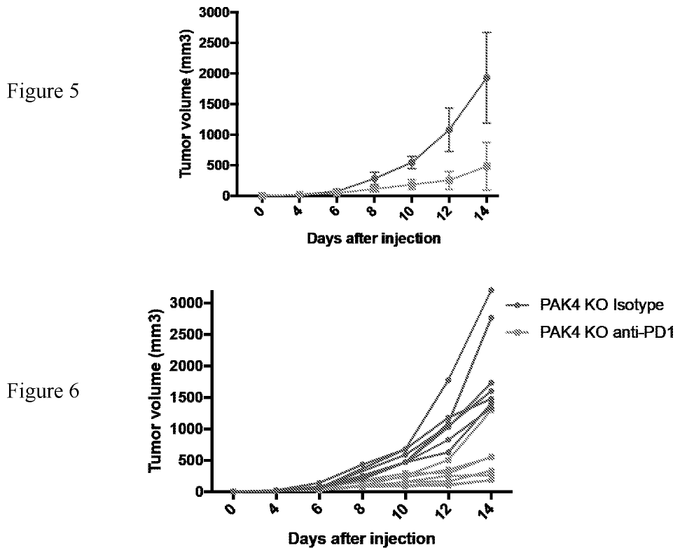

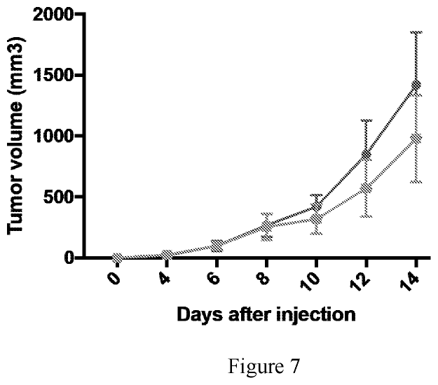

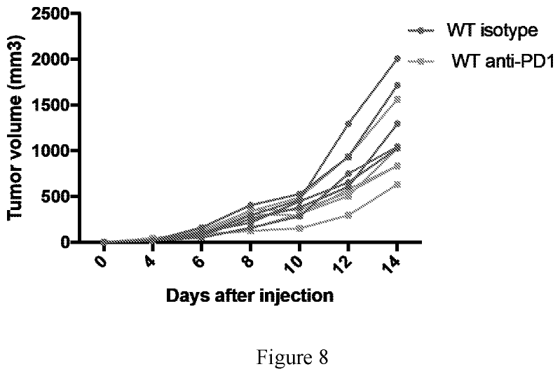

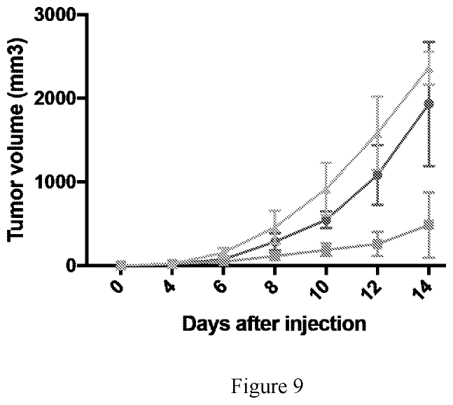

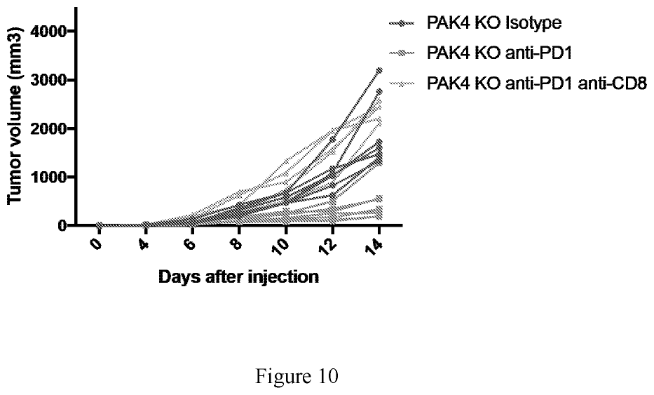

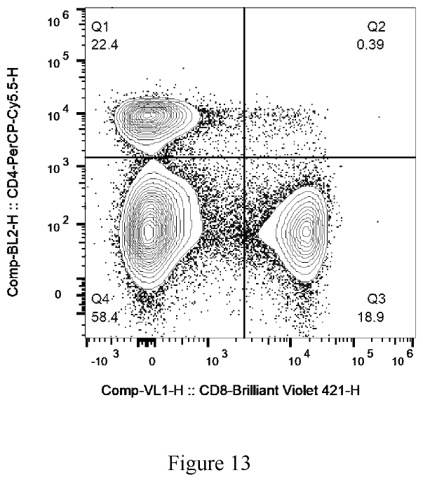

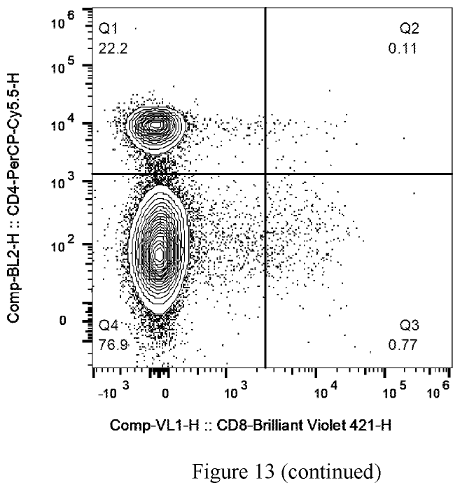

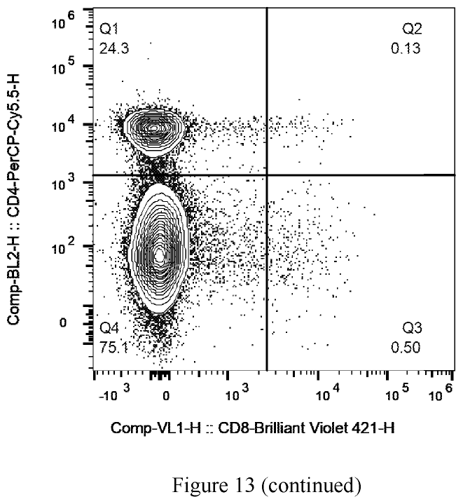

We next assessed PAK4 inhibition in the murine melanoma model B16, which does not respond to PD-1 blockade and lacks previous infiltration by tumor-specific lymphocytes¹⁸. We first generated a B16 PAK4 KO cell line using the gene editing tool CRISPR/Cas9 (data not shown). To assess anti-tumor efficacy of PD-1 blockade in the context of PAK4 deletion, we treated syngeneic C57BL/6 mice bearing B16 PAK4 KO or B16 WT tumors with a murine anti-PD-1. We observed anti-tumor activity of PD-1 blockade only in melanoma tumors lacking PAK4 expression (FIGS. 5-8). Of note, untreated B16 PAK4 KO tumors grew progressively, suggesting that although PAK4 deletion is important for response to PD-1 blockade therapy it is not necessarily sufficient by itself in the B16 model to trigger an antitumor immune response. To elucidate whether the observed response to anti-PD-1 was CD8-dependent, we depleted CD8 T cells in syngeneic C57BL/6 mice bearing B16 PAK4 KO tumors. CD8 depletion abrogated the anti-tumor activity of mouse anti-PD-1, demonstrating that PAK4 deletion sensitized melanoma B16 tumors to PD-1 blockade in a CD8 T cell-dependent manner (FIGS. 9-10, 13). These results suggest that genetic PAK4 deletion allows the priming and infiltration of tumor specific T cells that confer anti-tumor efficacy upon PD-1 blockade.

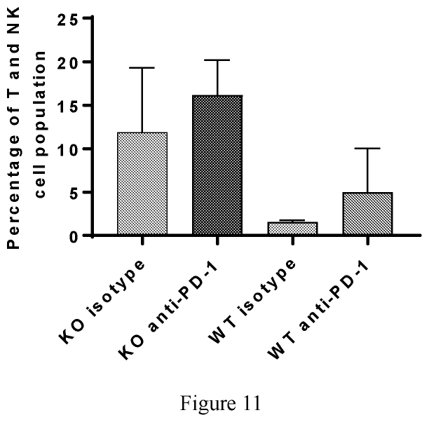

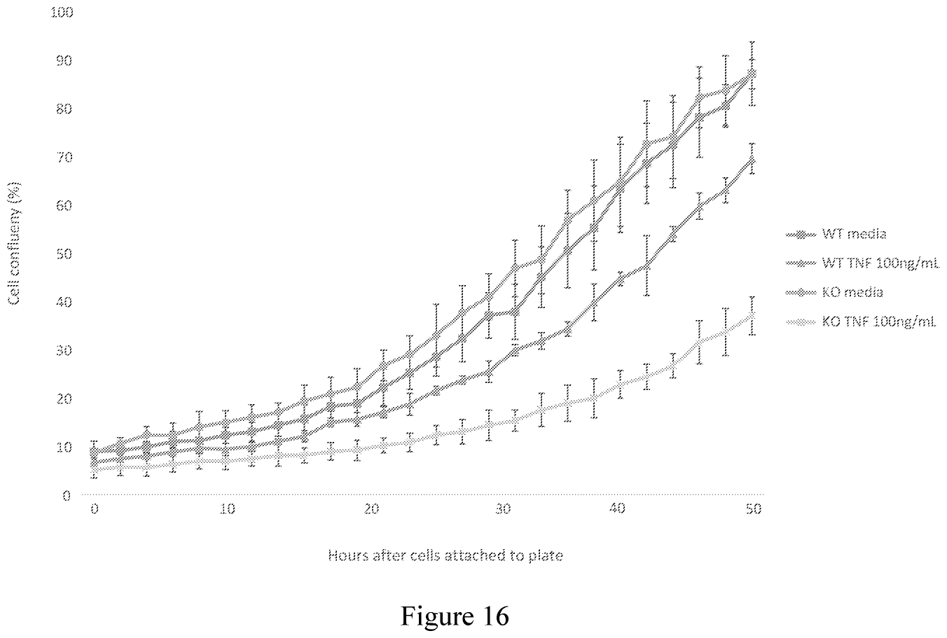

To test if PAK4 deletion facilitates immune cell infiltration, we performed immune profiling of tumor infiltrating immune cells using cytometry by time-of-flight (CyTOF) and identified a total of 16 independent cell clusters (data not shown). T cell population was defined by three clusters including a non-T regulatory CD4 T cell cluster, positive for CD3e, CD4, IFN-γ and Ki-67, a CD8 T cell cluster, positive for CD3e, CD8a, Tbet and Ki-67, and a general T cell cluster, positive for CD3e. A natural killer cluster positive for CD335 and CD161 was also identified. B16 PAK4 KO anti-PD-1 treated tumors presented increased infiltration of T and NK cells compared to B16 WT anti-PD-1 tumors (P=0.049, FIG. 11 and data not shown). Untreated B16 PAK4 KO tumors already presented increased T and NK cell infiltration compared to B16 WT untreated tumors (P=0.02, FIG. 11 and data not shown) although we did not observe consistent anti-tumor efficacy in the B16 PAK4 KO group (FIG. 14). Consistently, B16 PAK4 KO tumors had increased levels of T cells regardless of the treatment with murine anti-PD-1 (P=0.009, FIG. 15). In addition, we also observed that PAK4 KO B16 cells presented a decrease in cell proliferation upon stimulation with tumor necrosis factor alpha (TNF-α) (FIG. 16). This finding is consistent with previous data demonstrating that PAK4 is involved in the regulation of pro-survival and pro-apoptotic signals generated in response to TNF-α and that PAK4 deletion sensitizes tumor cells to TNF-α by favouring the apoptosis pathwayl^{9,20}. Therefore, this data supports and confirms the hypothesis that PAK4 depletion is sufficient to allow the infiltration of T cells and sensitizes tumors to PD-1 blockade.

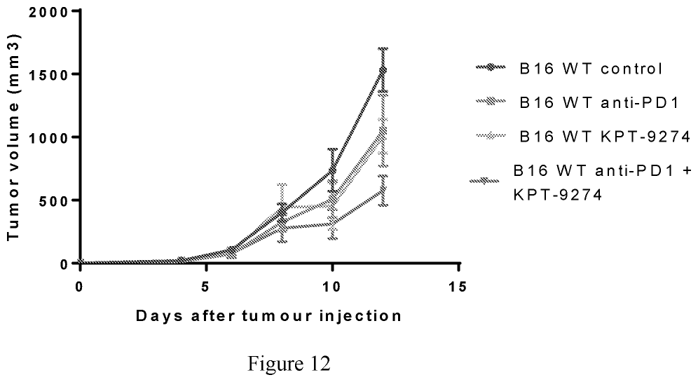

We next sought to determine if the PAK4 inhibitor, KPT-9274⁹⁻¹², recapitulates the anti-tumor effects previously observed. B16 murine melanoma tumors treated with anti-PD-1 in combination with KPT-9274 showed a stronger anti-tumor effect compared to anti-PD-1 (P=0.01, FIG. 12) and KPT-9274 monotherapy (P=0.0007, FIG. 12).

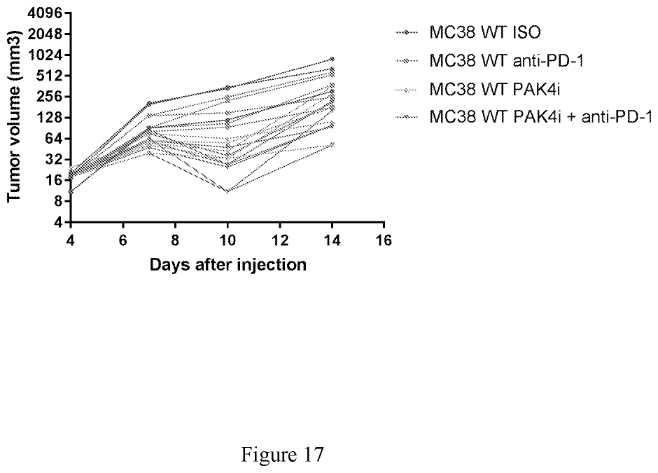

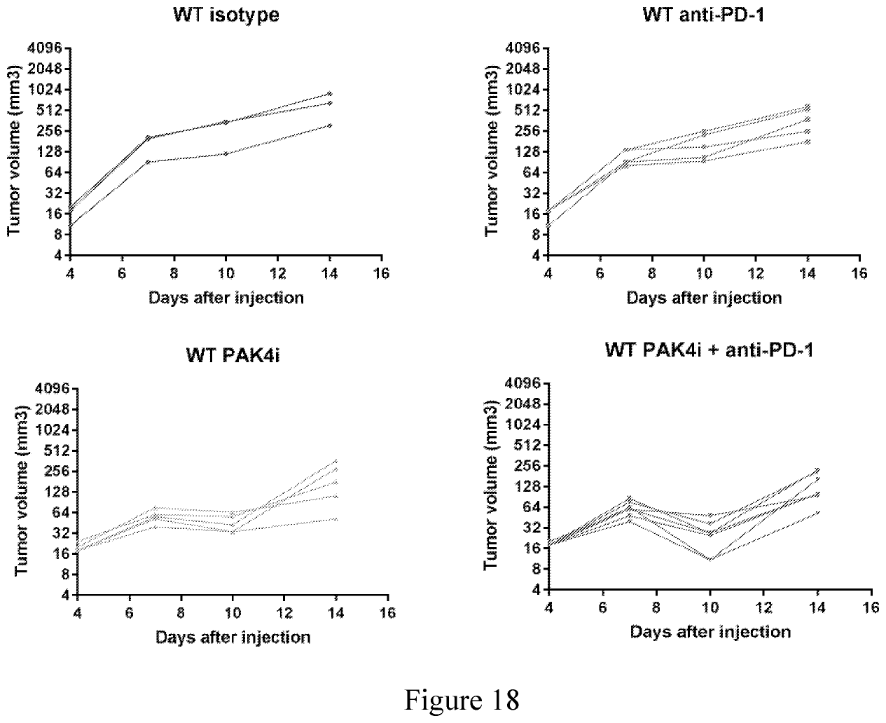

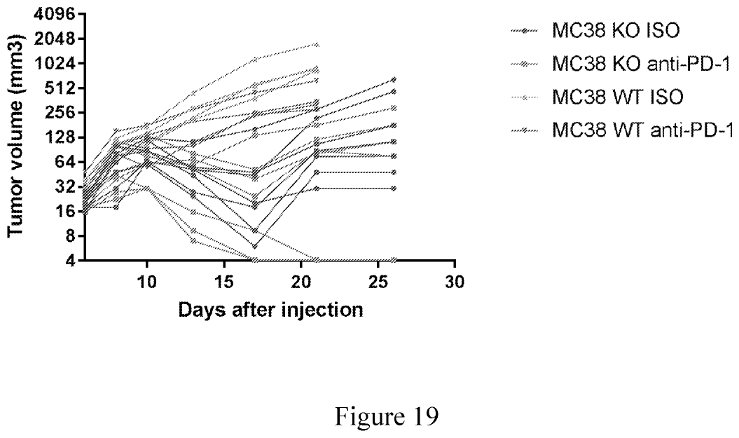

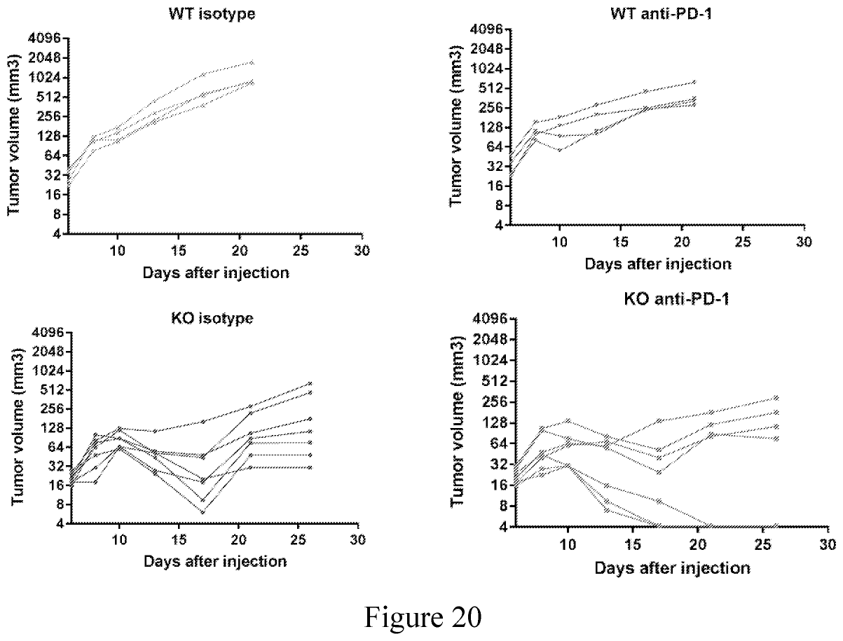

We reproduced these findings in a mouse colon adenocarcinoma model, MC38, which is a model of a cancer with high tumor mutation burden and is sensitive to PD-1 blockade²¹²². Consistent with being an immunogenic tumor model and PAK4 deletion per se facilitates T cell infiltration (FIG. 11), both MC38 WT tumors treated with either combination of PAK4 inhibitor and anti-PD-1, or PAK4 inhibitor alone, showed a decreased tumor growth compared to the anti-PD-1 monotherapy group (FIGS. 17-18). We generated a PAK4 KO subline of MC38 through CRISPR/Cas9 gene editing, and consistent with the results with the PAK4 inhibitor, the MC38 PAK4 KO tumors achieved tumor regression even in the absence of PD-1 blockade (FIGS. 19-20). Of note, only MC38 PAK4 KO anti-PD-1 treated tumors achieved complete regressions (n=3) suggesting that PD-1 blockade improves the anti-tumor T cell responses (FIG. 20). Altogether, this data suggests that PAK4 inhibition synergizes with anti-PD-1 treatment and presents a new strategy to overcome PD-1 blockade resistance.

Although the role of WNT signalling in immune cell exclusion in cancer is becoming clearer^{15,17,23} there was a lack of potential targets that could be inhibited to reverse the tumor intrinsic β-catenin effect on immune cell infiltration. This study presents an actionable mechanism to reverse WNT-related tumor-specific T cell exclusion using PAK4 inhibitors and overcome PD-1 blockade resistance. In addition, PAK4 inhibition may increase the sensitivity of cancer cells to the antitumor activity of T cells through increased pro-apoptotic effects of TNF-α.

### Example 3. Effect of PAK4 Deletion on Wnt Signalling

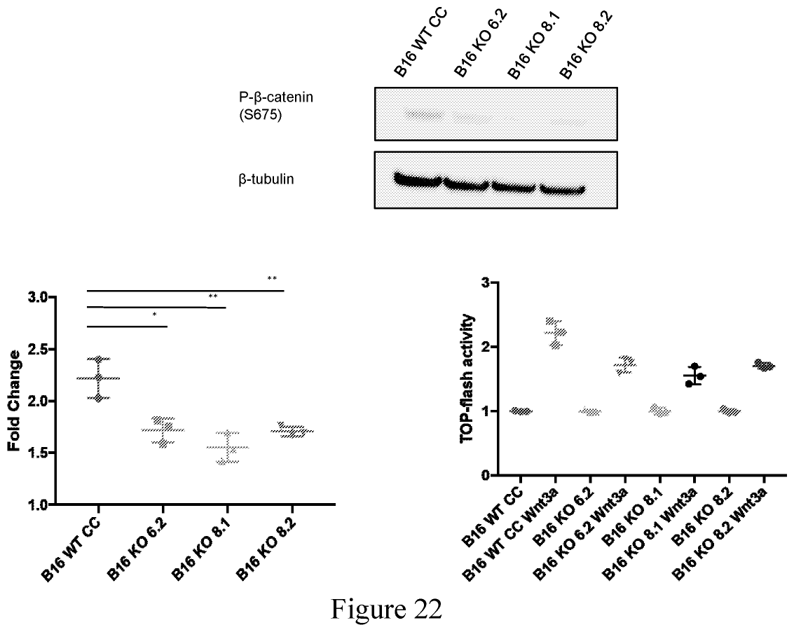

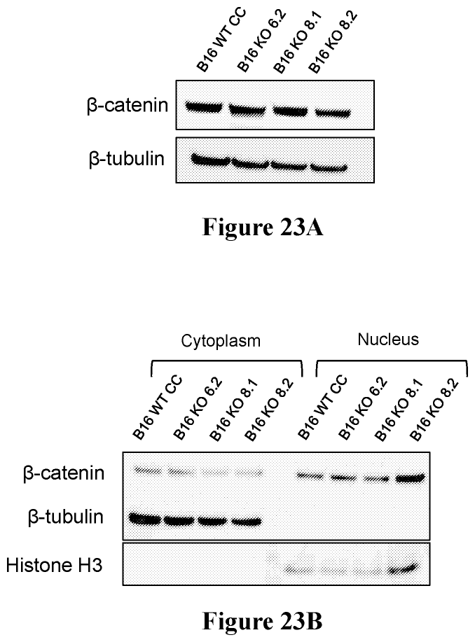

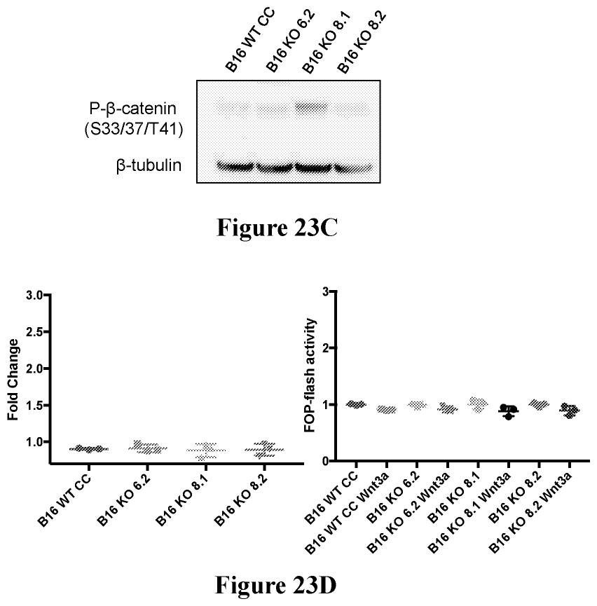

To directly investigate the impact of PAK4 deletion on Wnt signalling, PAK4 KO sublines of the murine melanoma B16 using CRISPR/Cas9 (FIG. 21) were first generated. The cell lines were then transfected with the Topflash luciferase reporter under the control of consensus TCF-binding sites^{33,34}. Whereas Wnt-3a treatment significantly induced the Topflash luciferase activity in B16 WT CRISPR control cells, the induction of Topflash luciferase activity by Wnt-3a was significantly reduced in PAK4 KO cells (FIG. 22). In contrast, Wnt-3a treatment did not induce the Fopflash luciferase activity, which is under the control of mutant TCF-binding sites, in both B16 WT cells and PAK4 KO cells (FIG. 23). Furthermore, although PAK4 deletion did not affect β-catenin protein levels nor cytoplasm/nuclear levels (FIG. 23), PAK4 deletion decreased β-catenin phosphorylation at S675 (FIG. 22). Taken together, these results indicate that the deletion of PAK4 impairs Wnt/β-catenin-mediated transcription.

### Example 4. Assessment of Anti-Tumour Efficacy of PD-1 Blockade in PAK4 Knockouts in B16 Cells

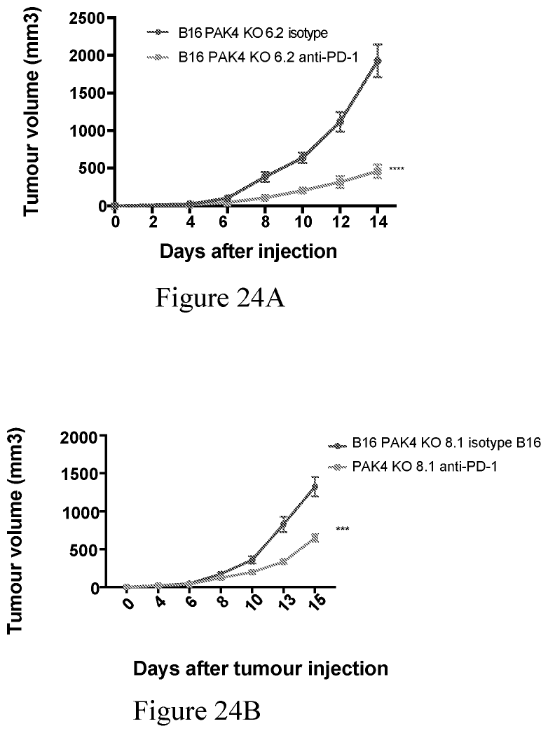

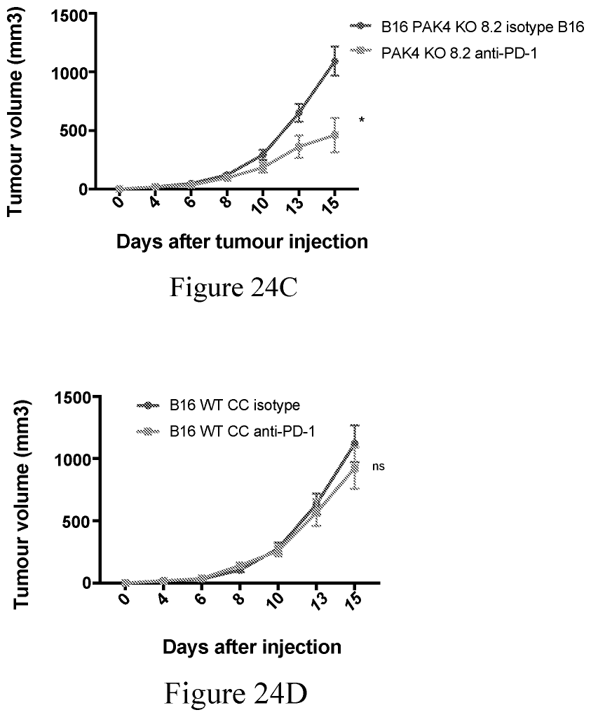

To assess anti-tumour efficacy of PD-1 blockade in the context of PAK4 deletion/inhibition, we treated syngeneic C57BL/6 mice bearing three different B16 PAK4 KO cell lines (6.2, 8.1, and 8.2) produced via CRISPR/Cas9 or B16 WT tumours with a murine anti-PD-1 antibody. We observed anti-tumour activity of PD-1 blockade only in melanoma tumours lacking PAK4 expression (FIG. 24). Of note, untreated B16 PAK4 KO tumours grew progressively, suggesting that although PAK4 deletion is a required step for response to PD-1 blockade therapy, it is not sufficient by itself in the B16 model to trigger an anti-tumour immune response.

### REFERENCES

1 Tumeh, P. C. et al. PD-1 blockade induces responses by inhibiting adaptive immune resistance. Nature 515, 568-571, doi:10.1038/nature13954 (2014).
2 Ayers, M. et al. IFN-gamma-related mRNA profile predicts clinical response to PD-1 blockade. J Clin Invest 127, 2930-2940, doi:10.1172/JCI91190 (2017).
3 Chen, P. L. et al. Analysis of Immune Signatures in Longitudinal Tumor Samples Yields Insight into Biomarkers of Response and Mechanisms of Resistance to Immune Checkpoint Blockade. Cancer Discov 6, 827-837, doi:10.1158/2159-8290.CD-15-1545 (2016).
4 Riaz, N. et al. Tumor and Microenvironment Evolution during Immunotherapy with Nivolumab. Cell 171, 934-949 e915, doi:10.1016/j.ce11.2017.09.028 (2017).
5 Spranger, S. & Gajewski, T. F. Impact of oncogenic pathways on evasion of antitumour immune responses. Nat Rev Cancer 18, 139-147, doi:10.1038/nrc.2017.117 (2018).
6 Rane, C. K. & Minden, A. P21 activated kinase signaling in cancer. Seminars in cancer biology, doi:10.1016/j.semcancer.2018.01.006 (2018).
7 Vershinin, Z., Feldman, M., Chen, A. & Levy, D. PAK4 Methylation by SETD6 Promotes the Activation of the Wnt/beta-Catenin Pathway. The Journal of biological chemistry 291, 6786-6795, doi:10.1074/jbc.M115.697292 (2016).
8 Li, Y. et al. Nucleo-cytoplasmic shuttling of PAK4 modulates beta-catenin intracellular translocation and signaling. Biochimica et biophysica acta 1823, 465-475, doi:10.1016/j.bbamcr.2011.11.013 (2012).
9 Takao, S. et al. Targeting the vulnerability to NAD(+) depletion in B-cell acute lymphoblastic leukemia. Leukemia 32, 616-625, doi:10.1038/1eu.2017.281 (2018).
10 Rane, C. et al. A novel orally bioavailable compound KPT-9274 inhibits PAK4, and blocks triple negative breast cancer tumor growth. Sci Rep 7, 42555, doi:10.1038/srep42555 (2017).
11 Abu Aboud, O. et al. Dual and Specific Inhibition of NAMPT and PAK4 By KPT-9274 Decreases Kidney Cancer Growth. Mol Cancer Ther 15, 2119-2129, doi:10.1158/1535-7163.MCT-16-0197 (2016).
12 Aboukameel, A. et al. Novel p21-Activated Kinase 4 (PAK4) Allosteric Modulators Overcome Drug Resistance and Stemness in Pancreatic Ductal Adenocarcinoma. Mol Cancer Ther 16, 76-87, doi:10.1158/1535-7163.MCT-16-0205 (2017).
13 Becht, E. et al. Estimating the population abundance of tissue-infiltrating immune and stromal cell populations using gene expression. Genome Biol 17, 218, doi:10.1186/s13059-016-1070-5 (2016).
14 Radu, M., Semenova, G., Kosoff, R. & Chernoff, J. PAK signalling during the development and progression of cancer. Nat Rev Cancer 14, 13-25 (2014).
15 Spranger, S., Bao, R. & Gajewski, T. F. Melanoma-intrinsic beta-catenin signalling prevents anti-tumour immunity. Nature 523, 231-235, doi:10.1038/nature14404 (2015).
16 Yun, C. Y. et al. p21-activated kinase 4 critically regulates melanogenesis via activation of the CREB/MITF and beta-catenin/MITF pathways. The Journal of investigative dermatology 135, 1385-1394, doi:10.1038/jid.2014.548 (2015).
17 Nsengimana, J. et al. beta-Catenin-mediated immune evasion pathway frequently operates in primary cutaneous melanomas. J Clin Invest 128, 2048-2063, doi:10.1172/JC195351 (2018).
18 Ueha, S. et al. Robust Antitumor Effects of Combined Anti-CD4-Depleting Antibody and Anti-PD-1/PD-L1 Immune Checkpoint Antibody Treatment in Mice. Cancer Immunol Res 3, 631-640, doi:10.1158/2326-6066.CIR-14-0190 (2015).
19 Li, X. & Minden, A. PAK4 functions in tumor necrosis factor (TNF) alpha-induced survival pathways by facilitating TRADD binding to the TNF receptor. The Journal of biological chemistry 280, 41192-41200, doi:10.1074/jbc.M506884200 (2005).
20 Li, Q. et al. p21-activated kinase 4 as a switch between caspase-8 apoptosis and NF-kappaB survival signals in response to TNF-alpha in hepatocarcinoma cells. Biochem Biophys Res Commun 503, 3003-3010, doi:10.1016/j.bbrc.2018.08.085 (2018).
21 Homet Moreno, B. et al. Response to Programmed Cell Death-1 Blockade in a Murine Melanoma Syngeneic Model Requires Costimulation, CD4, and CD8 T Cells. Cancer Immunol Res 4, 845-857, doi:10.1158/2326-6066.C₁R-16-0060 (2016).
22 Yadav, M. et al. Predicting immunogenic tumour mutations by combining mass spectrometry and exome sequencing. Nature 515, 572-576, doi:10.1038/nature14001 (2014).
23 Grasso, C. S. et al. Genetic Mechanisms of Immune Evasion in Colorectal Cancer. Cancer Discov 8, 730-749, doi:10.1158/2159-8290.CD-17-1327 (2018).
24 Kim, D., Langmead, B. & Salzberg, S. L. HISAT: a fast spliced aligner with low memory requirements. Nat Methods 12, 357-360, doi:10.1038/nmeth.3317 (2015).
25 Anders, S., Pyl, P. T. & Huber, W. HTSeq—a Python framework to work with high-throughput sequencing data. Bioinformatics 31, 166-169, doi:10.1093/bioinformatics/btu638 (2015).
26 Love, M. I., Huber, W. & Anders, S. Moderated estimation of fold change and dispersion for RNA-seq data with DESeq2. Genome Biol 15, 550, doi:10.1186/s13059-014-0550-8 (2014).
27 Subramanian, A. et al. Gene set enrichment analysis: a knowledge-based approach for interpreting genome-wide expression profiles. Proc Natl Acad Sci USA 102, 15545-15550, doi:10.1073/pnas.0506580102 (2005).
28 Ran, F. A. et al. Genome engineering using the CRISPR-Cas9 system. Nat Protoc 8, 2281-2308, doi:10.1038/nprot.2013.143 (2013).
29 Brinkman, E. K., Chen, T., Amendola, M. & van Steensel, B. Easy quantitative assessment of genome editing by sequence trace decomposition. Nucleic Acids Res 42, e168, doi:10.1093/nar/gku936 (2014).
30 Escuin-Ordinas, H. et al. COX-2 inhibition prevents the appearance of cutaneous squamous cell carcinomas accelerated by BRAF inhibitors. Mol Oncol 8, 250-260, doi:10.1016/j.molonc.2013.11.005 (2014).
31 Wei, S. C. et al. Distinct Cellular Mechanisms Underlie Anti-CTLA-4 and Anti-PD-1 Checkpoint Blockade. Cell 170, 1120-1133 e1117, doi:10.1016/j.cell.2017.07.024 (2017).
32 Chen, H. et al. Cytofkit: A Bioconductor Package for an Integrated Mass Cytometry Data Analysis Pipeline. PLoS Comput Biol 12, e1005112, doi:10.1371/journal.pcbi.1005112 (2016).
33 Chen, S. et al. Wnt-1 signaling inhibits apoptosis by activating beta-catenin/T cell factor-mediated transcription. J Cell Biol 152, 87-96 (2001).
34 Li, J. et al. LATS2 suppresses oncogenic Wnt signaling by disrupting beta-catenin/BCL9 interaction. Cell Rep 5, 1650-1663, doi:10.1016/j.celrep.2013.11.037 (2013).

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

1. A method of treating cancer in a subject, comprising:
(1) administering at least one PAK4 inhibitor to the subject; and
(2) administering at least one immunostimulatory agent to the subject.

2. (canceled)

3. The method of claim 1, wherein the PAK4 inhibitor is a small molecule chemical.

4. The method of claim 3, wherein the small molecule is at least one of KPT-9274, PF-3758309, IPA-3, FRAX1036, LCH-7749944, glaucarubinone, KY-04031, KY-040451-phenanthryl-tetrahydroisoquinoline derivatives, (−)-β-hydrastine, Inka1, GL 1196, or GNE-2861, optionally wherein the small molecule is KPT-9274.

5-9. (canceled)

10. The method of claim 1, wherein the PAK4 inhibitor is an RNA interference (RNAi) compound or an inhibitor of a microRNA, optionally wherein the microRNA is at least one of miR-145, miR-433, and miR-126.

11. (canceled)

12. The method of claim 1, wherein the immunostimulatory agent comprises an anti-PD1 antibody, an anti-PDL1 antibody, or an anti-CTLA4 antibody.

13. The method of claim 1, wherein the immunostimulatory agent comprises pembrolizumab (Keytruda), nivolumab (Opdivo), atezolizumab (Tecentriq), avelumab (Bavencio), durvalumab (Imfinzi), BMS-936559/MDX1105, PDR001/spartalizumab, GLS-010/AB-122, PF-06801591, BGB-a317, INCSHR-1210, TSR-042, JS-001, LY3300054, ipilimumab (Yervoy), tremelimumab, or AGEN-1884.

14. The method of claim 1, wherein the cancer is resistant to treatment with an immunostimulatory agent alone, optionally wherein the immunostimulatory agent is a checkpoint inhibitor.

15. The method of claim 1, wherein the cancer is cutaneous melanoma, microsatellite unstable cancers of any histology, head and neck carcinoma, lung carcinoma, renal cell carcinoma, bladder cancer, Merkel cell carcinoma, Hodgkin's lymphoma, gastroesophageal carcinoma, or hepatocellular carcinoma that are resistant to a prior therapy with anti-PD-1, anti-PD-L1, or anti-CTLA4 antibody therapy.

16. (canceled)

17. The method of claim 1, wherein the cancer is pancreatic cancer, colorectal cancer, breast cancer, adrenocortical carcinoma, testicular and germinal cell tumors, glioblastoma multiforme, uveal melanoma, thyroid cancer, endometrial cancer, ovarian cancer, cervical carcinoma, cholangiocarcinoma, mesothelioma, thymoma, a lymphoma, a leukemia, multiple myeloma or a sarcoma, with the PAK4 inhibitor given together with an immune checkpoint inhibitor along with standard of care chemotherapy and/or radiotherapy.

18. The method of claim 1, wherein the cancer is estrogen/progesterone receptor positive breast cancer, or prostate cancer, with the PAK4 inhibitor given together with an immune checkpoint inhibitor and hormone inhibitor therapy.

19. The method of claim 1, wherein the cancer is uveal melanoma, with the PAK4 inhibitor given together with an immune checkpoint inhibitor and one or more immune modulators such as a LAGS inhibitor, a TIM3 inhibitor, a TIGIT inhibitor, a CSF1R inhibitor, a PEGylated cytokine (optionally IL-2, IL-10, IFN), a GITR antibody, a A2AR inhibitor, an IDO inhibitor, or an antibody to OX40, CD40, or CD137/41BB.

20. The method of claim 1, wherein the cancer is pancreatic cancer, colorectal cancer, breast cancer, adrenocortical carcinoma, testicular and germinal cell tumors, glioblastoma multiforme, uveal melanoma, thyroid cancer, endometrial cancer, ovarian cancer, cervical carcinoma, cholangiocarcinoma, mesothelioma, thymoma, a lymphoma, a leukemia, multiple myeloma or a sarcoma, with the PAK4 inhibitor given together with an immune checkpoint inhibitor and one or more immune modulators such as a LAG3 inhibitor, a TIM3 inhibitor, a TIGIT inhibitor, a CSF1R inhibitor, a PEGylated cytokine (optionally IL-2, IL-10, IFN), a GITR antibody, a A2AR inhibitor, an IDO inhibitor, or an antibody to OX40, CD40, or CD137/41BB.

21. The method of claim 1, wherein the cancer is cutaneous melanoma, microsatellite unstable cancers of any histology, head and neck carcinoma, lung carcinoma, renal cell carcinoma, bladder cancer, Merkel cell carcinoma, Hodgkin's lymphoma, gastroesophageal carcinoma, or hepatocellular carcinoma, with the PAK4 inhibitor given together with an immune checkpoint inhibitor and one or more immune modulators such as a LAG3 inhibitor, a TIM3 inhibitor, a TIGIT inhibitor, a CSF1R inhibitor, a PEGylated cytokine (optionally IL-2, IL-10, IFN), a GITR antibody, a A2AR inhibitor, an IDO inhibitor, or an antibody to OX40, CD40, or CD137/41BB.

22-24. (canceled)

25. The method of claim 1, wherein the cancer is a solid tumor with the PAK4 inhibitor given together with the adoptive cell transfer of T cells modified to express a transgenic T cell receptor (TCR) or with the adoptive cell transfer of tumor-infiltrating lymphocytes (TILs).

26. (canceled)

27. The method of claim 1, wherein the cancer is PAK4+.

28. The method of claim 1, wherein the subject is a human subject.

29. A method of treating cancer in a subject, comprising administering a PAK4 inhibitor to the subject, wherein the cancer (1) has been determined to be substantially free or have a low baseline level of tumor-infiltrating T cells defined by having a density of less than 500 CD3+ or CD8+ T cells per mm square inside the tumor or at the invasive margin of the tumor when analyzed by immunohistochemistry or by mRNA expression of T cell genes or interferon gamma signaling genes or an equivalent T cell quantitation method; or (2) has been determined to have increased PAK4 expression relative to control, defined by measuring PAK4 protein expression by immunohistochemistry or an equivalent protein quantitation method or PAK4 mRNA expression by RNASeq, Nanostring, or an equivalent mRNA quantitation method.

30-65. (canceled)

66. A kit comprising at least one PAK4 inhibitor, at least one immunostimulatory agent, and instructions for use.

67-82. (canceled)

83. The method of claim 1, wherein the PAK4 inhibitor is a compound of Formula (I) 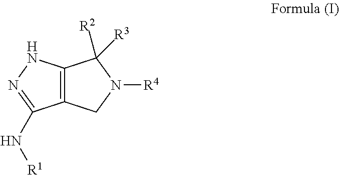
or a pharmaceutically acceptable salt thereof, wherein
R¹ is selected from the group consisting of —S(O)R^{a}, —S(O)₂R^{a}, C₁-C₁₂ alkyl, C₁-C₁₂ alkyl substituted by 1 to 6 R⁵, C₃-C₁₂ cycloalkyl, C₃-C₁₂ cycloalkyl substituted by 1 to 6 R⁵, C₂-C₁₂ alkenyl, C₂-C₁₂ alkenyl substituted by 1 to 6 R⁵, C₄-C₁₂ cycloalkenyl, C₄-C₁₂ cycloalkenyl substituted by 1 to 6 R⁵, C₂-C₁₂ alkynyl, C₂-C₁₂ alkynyl substituted by 1 to 6 R⁵, 3-12 membered heterocyclyl, 3-12 membered heterocyclyl substituted by 1 to 6 R⁵, C₁-C₆ aralkyl, C₁-C₆ aralkyl substituted by 1 to 6 R⁵, C₁-C₆ heteroaralkyl, C₁-C₆ heteroaralkyl substituted by 1 to 6 R⁵, phenyl, naphthyl, phenyl substituted by 1 to 6 R⁵, naphthyl substituted by 1 to 6 R⁵, 5-12 member heteroaryl, and 5-12 member heteroaryl substituted by 1 to 6 R⁵, wherein any two adjacent R⁵ together with the atoms to which they are attached may form a fused 4-7 member ring, and the said fused ring is optionally further substituted by 1-3 R^{f};
R² and R³ are each independently selected from the group consisting of —H, C₁-C₆ perfluoroalkyl, C₁-C₆ alkyl, C₃-C₆ cycloalkyl, —(C₁-C₃ alkylene)-(C₃-C₆ cycloalkyl), C₂-C₆ alkenyl, C₂-C₆ alkynyl, C₁-C₆ alkoxy, -(L)ₘ-halide, -(L)ₘ-CN, -(L)ₘ-OH, -(L)ₘ-NH₂, -(L)ₘ-(C₁-C₆ monoalkylamino) and -(L)ₘ-(C₂-C₈ dialkylamino), provided that R² and R³ are not both H; or R² and R³ may form a ring selected from C₃-C₆ cycloalkyl, C₄-C₆ cycloalkenyl and 3-6 member heterocyclyl, the said ring is optionally further substituted by 1 to 2 groups selected from C₁-C₃ alkyl, C₁-C₃ perfluoroalkyl, C₁-C₃ alkoxy, oxo, —(C₁-C₃ alkylene)ₘ-halide, —(C₁-C₃ alkylene)ₘ-CN, —(C₁-C₃ alkylene)ₘ-OH, —(C₁-C₃ alkylene)ₘ-NH₂, —(C₁-C₃ alkylene)ₘ-(C₁-C₆ monoalkylamino) and —(C₁-C₃ alkylene)ₘ-(C₂-C₈ dialkylamino);
R⁴ is selected from the group consisting of R^{a}, —C(O)R^{a}, —C(O)NR^{a}R^{b}, —C(O)OR^{a}, —C(O)CH(R^{t})R^{a}, —C(O)NHCH(R^{a})R^{b}, —C(O)OCH(R^{a})R^{b}, —C(O)CH(R^{t})CH(R^{a})R^{b}, —C(O)SR^{a}, —S(O)R^{a}, —S(O)NR^{a}R^{b}, —S(O)OR^{a}, —S(O)₂R^{a}, —S(O)₂NR^{a}R^{b} and —S(O)₂OR^{a}, wherein R^{t} is H or C₁-C₃ alkyl; each R⁵ is independently selected from the group consisting of R^{c}, -(L)ₘ-halide, -(L)ₘ-CN, -(L)ₘ-C(O)R^{c}, -(L)ₘ-C(O)O R^{c}, -(L)ₘ-C(O)NR^{c}R^{d}, -(L)ₘ-C(O)SR^{c}, -(L)ₘ-OR^{c}, -(L)ₘ-OC(O)R^{c}, -(L)ₘ-OC(O)NR^{c}R^{d}, -(L)ₘ-O—C(O)OR^{c}, -(L)ₘ-NO₂, -(L)ₘ-NR^{c}R^{d}, -(L)ₘ-N(R^{c})C(O)R^{d}, -(L)ₘ-N(R^{c})C(O)OR^{d}, -(L)ₘ-NR^{c}S(O)R^{d}, -(L)ₘ-NR^{c}S(O)OR^{d}, -(L)ₘ-NR^{c}S(O)₂R^{d}, -(L)ₘ-NR^{c}S(O)₂OR^{d}, -(L)ₘ-SR^{c}, -(L)ₘ-S(O)R^{c}, -(L)ₘ-S(O)OR^{c}, -(L)ₘ-S(O)₂R^{c}, -(L)ₘ-S(O)₂OR^{c}, -(L)ₘ-S(O)NR^{c}R^{d}, -(L)ₘ-S(O)₂NR^{c}R^{d}, -(L)ₘ-O-L-NR^{c}R^{d}, -(L)ₘ-O-L-OR^{c} and -(L)ₘ-NR^{c}-L-OR^{d};
each R^{a}, R^{b}, R^{c}, and R^{d} is independently selected from the group consisting of H, -(L)ₘ-(C₁-C₆ perfluoroalkyl), C₁-C₁₂ alkyl, —(C₁-C₃ alkylene)ₘ-(C₃-C₁₂ cycloalkyl), —(C₃-C₅ cycloalkylene)ₘ-(C₂-C₁₂ alkenyl), -(L)ₘ-(C₄-C₁₂ cycloakenyl), —(C₃-C₅ cycloalkylene)ₘ-(C₂-C₁₂ alkynyl), -(L)ₘ-(3-12 member heterocyclyl), -(L)ₘ-(phenyl), -(L)ₘ-(naphthyl), and -(L)ₘ-(5-12 member heteroaryl), wherein each R^{a}, R^{b}, R^{c} and R^{d} is independently optionally further substituted by 1-6 R^{f};
R^{a} and R^{b}, or R^{c} and R^{d}, together with the atom to which they are attached, may optionally form a ring selected from 3-12 member heterocyclyl and 5-12 member heteroaryl, the said ring is optionally further substituted by 1-6 R^{f};
each R^{f} is independently selected from oxo, —(C₁-C₃ alkylene)ₘ-(C₁-C₆ perfluoalkyl), C₁-C₁₂ alkyl, C₂-C₆ alkenyl, C₂-C₆ alkynyl, —(C₁-C₃ alkylene)ₘ-(C₃-C₇ cycloalkyl), —(C₁-C₃ alkylene)ₘ-(3-7 member heterocyclyl), —(C₁-C₃ alkylene)ₘ-(5-7 member heteroaryl), -(L)ₘ-halide, -(L)ₘ-CN, -(L)ₘ-C(O)R^{k}, -(L)ₘ-C(O)OR^{k}, -(L)ₘ-C(O)NR^{k}R^{j}, -(L)ₘ-OR^{k}, -(L)ₘ-OC(O)R^{k}, -(L)ₘ-NO₂, -(L)ₘ-NR^{k}R^{j}, -(L)ₘ-N(R^{k})C(O)R^{j}, -(L)ₘ-O-L-NR^{k}R^{j}, -(L)ₘ-SR^{k}, -(L)ₘ-S(O)R^{k}, -(L)ₘ-S(O)₂R^{j}R^{k}, wherein each R^{f} is independently optionally further substituted by 1-3 groups selected from C₁-C₃ alkyl, halide and C₁-C₃ perfluoroalkyl;
each R^{k} and R^{j} is independently —H, —OH, C₁-C₃ perfluoroalkyl, C₁-C₆ alkyl, C₂-C₆ alkenyl, C₃-C₆ alkynyl, —(C₁-C₃ alkylene)ₘ-(C₃-C₆ cycloalkyl) or —(C₁-C₃alkylene)ₘ-(3 to 6 member heterocyclyl), R^{k} and R^{j} may optionally form a ring selected from 3-7 member heterocyclyl and 5-7 member heteroaryl, with said ring optionally further substituted by 1 to 2 groups selected from C₁-C₃ alkyl, C₁-C₃ perfluoroalkyl, C₁-C₃ alkoxy, oxo, —(C₁-C₃ alkylene)ₘ-halide, —(C₁-C₃ alkylene)ₘ-CN, —(C₁-C₃ alkylene)ₘ-OH, —(C₁-C₃ alkylene)ₘ-NH₂, —(C₁-C₃ alkylene)ₘ-(C₁-C₆ monoalkylamino) and —(C₁-C₃ alkylene)ₘ-(C₂-C₈ dialkylamino);
each L is independently a bivalent radical selected from —(C₁-C₆ alkylene)-, —(C₃-C₇ cycloalkylene)-, —(C₁-C₆ alkylene)-(C₃-C₇ cycloalkylene)- and —(C₃-C₇ cycloalkylene)-(C₁-C₆ alkylene)-;
each m is independently 0 or 1; and
n is 1, 2, or 3.

84. The method of claim 1, wherein the PAK4 inhibitor is a compound of Formula (II) 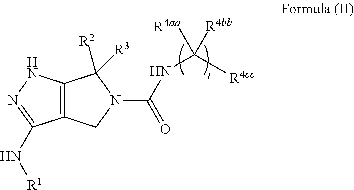
or a pharmaceutically acceptable salt thereof, wherein
R¹ is 9 or 10-membered bicyclic heteroaryl optionally substituted with 1, 2, or 3 independent occurrences of C₁-C₆ alkyl;
R² and R³ are each independently selected from C₁-C₆ alkyl;
R^{4aa} and R^{4bb} are each independently selected from the group consisting of —H, phenyl, naphthyl, and C₁-C₆ aralkyl;
R^{4cc} is —NR^{aa}R^{bb}; R^{aa} and R^{bb} are each independently selected from the group consisting of —H, C₁-C₆ alkyl, C₂-C₆ alkenyl, C₂-C₆ alkynyl, C₃-C₁₂ cycloalkyl, C₄-C₁₂ cycloalkenyl, 3-12 membered heterocyclyl, and C₁-C₆ aralkyl; and
t is an integer selected from the group consisting of 1, 2, and 3.
